(19) United States
(12) Patent Application Publication
(10) Pub. No.: US 2023/0145822 A1
(43) Pub. Date: May 11, 2023

(54) SMALL MOLECULES INDUCING THE DEGRADATION OF THE CELLULAR PRION PROTEIN

(71) Applicants: ISTITUTO NAZIONALE DI FISICA NUCLEARE, Frascati, Roma (IT); UNIVERSITA' DEGLI STUDI DI PERUGIA, Perugia (IT); UNIVERSITA' DEGLI STUDI DI TRENTO, Trento (IT); FONDAZIONE TELETHON, Roma (IT)

(72) Inventors: Emiliano BIASINI, Trento (IT); Maria Letizia BARRECA, Perugia (IT); Pietro FACCIOLI, Frascati, Roma (IT); Ines MANCINI, Trento (IT)

(21) Appl. No.: 17/914,872

(22) PCT Filed: Mar. 29, 2021
(86) PCT No.: PCT/IB2021/052586

§ 371 Date: Sep. 27, 2022

(30) Foreign Application Priority Data
Mar. 27, 2020 (IT) 102020000006517

(51) Int. Cl. C07D 221/04; A61P 43/00; C07D 471/04; A61K 45/06

(52) U.S. Cl. CPC C07D 221/04; A61P 43/00; C07D 471/04; A61K 45/06

(57) ABSTRACT

Chemical entities are capable of inducing the degradation of the cellular prion protein (PrP^{C}) identified with the pharmacological protein inactivation by folded intermediate targeting (PPI-FIT) methodology.

The present invention finds application in the medical field and, in particular, in the treatment of disorders related to the cellular prion protein (PrP^{C}).

### BACKGROUND OF THE INVENTION

Aging is related to multiple molecular, cellular and functional changes, which particularly affect the integrity of the nervous system.

One fundamental process altered by aging is protein folding. When proteins misfold, they acquire alternative, β-sheet rich conformations capable of triggering a cascade of molecular events, ultimately leading to neuronal dysfunction and death. Disease-associated aggregates, often referred to as amyloid fibrils, can arise from proteins with no sequence or structural homology, and be associated to a wide range of syndromes and clinical phenotypes; examples include common disorders such as Parkinson's and Alzheimer's diseases, as well as rarer disorders such as amyotrophic lateral sclerosis or prion diseases.

Prion diseases, which include Creutzfeldt-Jakob disease (CJD), Gerstmann-Straussler-Scheinker (GSS) syndrome and fatal familial insomnia (FFI), are pathologies linked to protein misfolding that can manifest in sporadic, inherited or transmissible or acquired fashion.

Prion diseases, also known as transmissible spongiform encephalopathies, are associated with the conformational conversion of PrP^{C}, an endogenous glycosyl-phosphatidyl-inositol (GPI)-anchored cell-surface glycoprotein, into a misfolded isoform called “scrapie form of PrP” (or PrPSc) that accumulates in the central nervous system of affected individuals.

PrPSc is an infectious protein (prion) lacking any detectable information-coding nucleic acid, that is capable of propagating like an infectious agent (prion) by directly binding to PrP^{C} and triggering its conformational rearrangement into new PrPSc molecules, hencecatalyzing the structural conversion of its physiological counterpart.

Despite these peculiar features, increasing evidence arising from genetic, biophysical and biochemical studies indicate that the pathogenic mechanisms operating in prion diseases may lie at the root of the neurodegenerative pathways occurring in several other disorders.

For example, it is becoming evident that PrP^{C} plays a dual role in prion diseases, being both a substrate for PrPSc replication, and a mediator of its toxicity.

This concept was unexpectedly expanded by data involving oligomeric assemblies of the amyloid β (AB) peptide and of the protein alpha-synuclein, which are believed to cause the synaptotoxicity underlying the cognitive decline in Parkinson's and Alzheimer's diseases, respectively. In fact, different studies have provided evidence that PrP^{C} could mediate the toxicity of oligomeric assemblies of Aβ and alpha-synuclein, suggesting that misfolded assemblies of several different pathogenic proteins could exert their detrimental effects by blocking, enhancing or altering the normal activity of PrPC. In addition, research in even more distant fields have underlined surprising roles for PrP^{C} in several physiological and disease contexts outside the brain, such as myelin disorders, auto-immune diseases and cancer (Kuffer, A., et al., The prion protein is an agonistic ligand of the G protein-coupled receptor Adgrg6. Nature, 2016; Mabbott, N.A., Immunology of Prion Protein and Prions. Prog Mol Biol Transl Sci, 2017; Hirsch, T. Z., S. Martin-Lanneree, and S. Mouillet-Richard, Functions of the Prion Protein. Prog Mol Biol Transl Sci, 2017).

Importantly, a great deal of data indicates that inhibiting the function of PrP^{C} in patients may be the most effective strategy to counteract neurotoxic events without causing major detrimental side effects.

This hypothesis was recently reinforced by the identification of loss-of-function alleles in healthy individuals, supporting the safety of therapeutic strategies aimed at targeting PrP^{C}.

Thus, compounds capable of modulating the expression and/or activity of PrP^{C} could provide a completely new therapeutic perspective for diseases related to the cellular prion protein (PrP^{C}) or interactors of PrP^{C} or toxic signaling pathways involving PrP^{C}.

These include neurodegenerative disorders, such as sporadic, inherited or acquired prion diseases, Alzheimer's disease, Parkinson's diseases and other α-synucleinopathies; neuroinflammatory disorders and demyelinating diseases, such as multiple sclerosis; cancer, in particular glioblastoma, gastric cancer, breast cancer, colon cancer.

Experimental evidence supports the rationale of employing state-of-the-art computational technologies to identify new therapies for a variety of human pathologies.

Such a process, often referred to as computer-aided drug discovery, relies on the precise information regarding the three-dimensional structure of proteins (the so called “native conformation”) associated with the disease of interest, and optionally how such structure could be altered to generate misfolded isoforms.

Elucidating the molecular mechanisms underlying folding and misfolding pathways of disease-associated proteins is a pre-requisite for designing effective therapies.

However, experimental approaches aimed at addressing the dynamics of protein folding are seriously affected by the tradeoff between temporal and spatial resolutions of available biophysical techniques. Computer-based technologies, such as Molecular Dynamics (MD) simulations, could in principle overcome these limitations and help to predict the evolution in time of the conformation of proteins. In practice, however, classical MD simulations are not applicable to study many fundamental molecular processes, such as the cascade of events underlying the folding of a typical, biologically-relevant polypeptide bigger than 100 amino acids. The reason is that the timescales, which can be simulated by MD, even on the most powerful supercomputers, are still orders of magnitude shorter than those at which major structural transitions take place.

Prior-art documents WO 2012/054535 and JP 2005 120002 disclose compounds that are capable of blocking the conformational transition from PrPC (in its mature, native state) into PrPSc by acting on a folding intermediate appearing along the misfolding conversion mechanism from PrPC to PrPSc.

### SUMMARY OF THE INVENTION

The inventors of the present patent application have surprisingly found that in order to overcome the limitations of the prior-art methods, an innovative experimental paradigm for drug discovery could be applied, named “pharmacological protein inactivation by folding intermediate targeting” (PPI-FIT), capable of identifying small organic ligands for the most kinetically- and thermodynamically-relevant folding intermediate of a protein in order to stabilize such conformation, promote its degradation by the cellular quality control machinery and thus inhibit the formation of mature protein.

In contrast with the prior-art disclosing compounds capable of blocking the transition from PrP, which is the mature and native state, into PrPSc by acting on a misfolding conversion mechanism, the inventors have identified molecules characterized by different chemical scaffolds, which are capable of targeting a folding intermediate of the prion protein (PrP^{C}). As a consequence, they promote the lysosomal degradation of the PrP polypeptide from the endoplasmic reticulum. These compounds have the remarkable ability to suppress the maturation of the PrP into its native conformation. As a final result, the PrPSc formation and therefore its replication are inhibited.

Accordingly, the discovered scaffolds and compounds are useful for the treatment of diseases or disorders related to the cellular prion protein (PrP^{C}).

These include neurodegenerative disorders, such as sporadic, inherited or acquired prion diseases, Alzheimer's disease, Parkinson's diseases and other α-synucleinopathies; neuroinflammatory disorders and demyelinating diseases, such as multiple sclerosis; cancer, in particular glioblastoma, gastric cancer, breast cancer, colon cancer.

### OBJECT OF THE INVENTION

A first object of the invention is represented by the compounds within the general formulas of claim 1, and dependent claims, capable of inducing the degradation of the cellular prion protein and which are identified using the PPI-FIT methodology.

Specific compounds within the present invention represent particular embodiments.

In a second object, the present invention discloses pharmaceutical compositions comprising the compounds of the invention.

In a third object, the invention discloses compounds within the general formula of the invention, the specific compounds as well as pharmaceutical compositions comprising them, for use as a medicament.

In particular, the compounds of the invention are disclosed for use in the treatment of diseases or disorders related to the cellular prion protein (PrP^{C}) or interactors of PrP^{C} or toxic signaling pathways involving PrP^{C}, including neurodegenerative disorders, such as sporadic, inherited or acquired prion diseases, Alzheimer's disease, Parkinson's diseases and other α-synucleinopathies; neuroinflammatory disorders and demyelinating diseases, such as multiple sclerosis; cancer, in particular glioblastoma, gastric cancer, breast cancer, colon cancer.

In a further object, it is disclosed a method for the treatment of diseases and/or disorders related to the cellular prion protein (PrP^{C}) or interactors of PrP^{C} or toxic signaling pathways involving PrP^{C} comprising the administration of a compound of the invention or of a pharmaceutical composition comprising a compound of the invention to a patient in need thereof.

A compound of the invention may optionally be administered in combination with further therapeutic molecules.

### BRIEF DESCRIPTION OF THE FIGURES

FIG. 1: 3D structures of the globular, C-terminal domain of native PrP^{C} (left) and its folding intermediate FI-PrP (right). A unique druggable pocket (right) was identified in FI-PrP. Such pocket is not accessible in native PrP^{C} (left).

FIG. 2: Representation of PrP structure. A. schematic representation of the structure of human PrP. The protein is organized as follows: a signal peptide (residues 1-22), precedes five histidine-containing octapeptide repeats (residues 51-89), which can bind divalent ions. The central region includes a highly conserved hydrophobic domain (residues 111-134). The C-terminal, globular domain (solved by NMR, PDB 1QLX, shown in panel B) includes two short anti-parallel β-strands (residues 127-129; and 166-168) and three a-helices (H1, H2 and H3, residues 143-152, 171-191 and 199-221, respectively). A C-terminal peptide (residues 232-253) is removed to allow the attachment of a glycosylphosphatidylinositol (GPI) moiety, which anchors the protein to the outer leaflet of the plasma membrane. The globular domain also contains two N-linked oligosaccharide chains (at Asn-181 and Asn-197) and a disulfide bond between residues 179 and 214.

FIG. 3: unique druggable pocket identified in the PrP folding intermediate, highlighted in grey. The box shows the specific residues defining the site.

FIG. 4: Synthetic scheme of SM875. Reagents and conditions: (a) NaHCO₃aqueous solution; (b) ethanol reflux, 2 h; (c) 1:1 methanol/aqueous 2M NaOH, reflux 1 h; (d) 180° C., 10 min; 61%; (e) ethanol reflux 2.5 h, HPLC purification, 25%. Arbitrary numbering of synthetic intermediate [1-(4-bromophenyl)-1H-pyrazol-5-amine] is used for 1H-NMR assignment: [δH 7.58(d, J 8.7 Hz, H-3′ and H-5′), 7.47(d, J 8.7 Hz, H-2′ and H-6′), 7.41(s, H-3), 5.62 (s, H-4)].

FIG. 5: Structural characterization of SM875. A. ¹H-NMR spectrum of SM875 (upper panel, 400 MHz) in CDCl₃ with residual CHCl₃ at 7.25 ppm. 13 C-NMR spectrum (lower panel, 100 MHz) of SM875 in CDCl₃. B. ESI-MS analysis by direct infusion of a methanol solution of SM875 spectrum in negative ion mode with m/z 412.1/414.1 corresponding to [M-H]⁻ signal (upper panel); fragmentation showing m/z 397.1/399.1 (lower panel); C. IR spectrum of the final product SM875. Relevant bands (cm⁻¹: 1681s, 1543s, 1515s, 1490s, 1274s, 991m, 828m, 728 vs; D. (i) Upper panel shows LC-ESI-MS analysis of SM875 separated by an RP18 column in acetonitrile/water 70:30 and detected at 254 nm (flow 1 mL min⁻¹). Lower panel shows extracted-ion chromatogram corresponding to (ii) m/z 414/416 [M+H]⁺ ion for C₁₉H₁₇⁷⁹BrN₃O₃/C₁₉H₁₇⁸¹BrN₃O₃.

FIG. 6: A. Chemical structure of SM875. B-C. Dose-response treatment of SM875 in HEK293 cells (ATCC, CRL-1573) stably transfected with mouse PrP^{C} or NEGR-1, as detected by Western blots using with specific anti-PrP^{C} (B) or anti-NEGR-1 (C) antibodies. D. Dose-response treatment of SM875 in human breast cancer cell line ZR-75 endogenously expressing human PrP^{C}, as detected by probing blots with a specific anti-PrP^{C} antibody.

FIG. 7: SM785 effect on post-translational processing of PrP^{C}. HEK293 cells stably expressing mouse PrP^{C} were exposed to different concentrations of SM875 (shown) or vehicle (DMSO, volume equivalent) for 24 h, lysed, treated with the enzyme PNGase-F to remove sugar moyeties, and analyzed by Western blotting.

FIG. 8: Effect of SM875 on cellular localization of PrP^{C}. Immunofluorescence analysis of HEK293 cells stably expressing EGFP-tagged PrP^{C} incubated with SM875 (B and C) or vehicle (DMSO) control (A).

FIG. 9: A. Molecular structure of SM875-derivative SM898. B. Cell viability assay in PrP^{C}-expressing HEK293 cells, as assayed by MTT essay. C. Effects of SM875 and SM898 on PrP^{C} expression, as assayed by Western blotting.

FIG. 10: Analysis of effects of PPI-FIT-derived compound SM930 on PrP^{C} expression. A. Chemical structure of SM930; B. Cell viability assay in HEK293 (left panel) or ZR-75 cells (right panel), as assayed by MTT essay, showing that SM930 induces severe cell death at the highest concentration tested (30 μM); C. Representative Western blotting images and quantification graphs showing that SM930 significantly suppresses mouse PrP^{C} expression at the highest concentration (30 μM), but not the control protein NEGR-1, when tested in stably-transfected HEK293 cells; D. Representative Western blotting images and quantification graphs showing that SM930 significantly suppresses human PrP^{C} expression (starting from the concentration of 3 μM), but not the control protein Thy-1, when tested in untransfected ZR-75 human breast cancer cells. Statistical differences were estimated by one-way Anova, Dunnet post-hoc test. p values are: *<0.05, **<0.01, ***<0.001.

FIG. 11: Analysis of effects of PPI-FIT-derived compound SM940 on PrP^{C} expression. A. Chemical structure of SM940; B. Cell viability assay in HEK293 or ZR-75 cells, as assayed by MTT, showing that SM940 does not induce detectable cell death at the concentrations tested (indicated); C. Representative Western blotting images and quantification graphs showing that SM940 significantly suppresses mouse PrP^{C} expression starting from 3 μM, but not the control protein NEGR-1, when tested in stably-transfected HEK293 cells; D. Representative Western blotting images and quantification graphs showing that SM940 significantly suppresses human PrP^{C} expression (starting from the concentration of 3 μM), but not the control protein Thy-1, when tested in untransfected ZR-75 human breast cancer cells. Statistical differences were estimated by one-way Anova, Dunnet post-hoc test. p values are: *<0.05, **<0.01.

FIG. 12: Analysis of effects of PPI-FIT-derived compound SM950 on PrP^{C} expression. A. Chemical structure of SM950; B. Cell viability assay in HEK293 or ZR-75 cells, as assayed by MTT, showing that SM950 induces detectable cell death at some of the concentrations tested (indicated); C. Representative Western blotting images and quantification graphs showing that SM950 significantly suppresses mouse PrP^{C} expression starting from 10 μM, but not the control protein NEGR-1, when tested in stably-transfected HEK293 cells; D. Representative Western blotting images and quantification graphs showing that SM950 significantly suppresses human PrP^{C} expression (starting from the concentration of 1 μM), but not the control protein Thy-1, when tested in untransfected ZR-75 human breast cancer cells. Statistical differences were estimated by one-way Anova, Dunnet post-hoc test. p values are: *<0.05, **<0.01, ***<0.001.

FIG. 13: Real-Time PCR analysis of PrP^{C} mRNA levels upon compound treatment. SM875 does not decrease PrP^{C} mRNA levels in: A, stably transfected HEK293 cells; B, untransfected human breast cancer cells ZR-75; or C, L929 mouse fibroblasts. Similarly, compounds SM940 (D) and SM950 (E) do not suppress mouse PrP^{C} mRNA levels in HEK293 cells. These results demonstrate that the different compounds do not decrease the amount of PrP^{C} mRNA, thus demonstrating that they act at a post-translational level. Interestingly, a significant increase of PrP^{C} mRNA was occasionally detected for some cells/concentrations, possibly indicating a compensatory response of cells to the induced decrease of PrP^{C} at the protein level. Statistical differences were estimated by one-way Anova, Dunnet post-hoc test. p values are: *<0.05.

FIG. 14: PPI-FIT-derived compounds SM875 and SM940 induce the aggregation of recombinant PrP^{C} in a temperature-dependent fashion. A. Schematic of the experiment. In order to observe a direct effect of the molecules identified on PrP^{C} in vitro, an experimental paradigm was designed in which natively folded PrP^{C} is heated at different temperatures with the hope to provide enough energy to allow its conformational transition to the predicted folding intermediate (FI-PrP). If the folding intermediate appears as a consequence of the raising temperature, then the inventors predicted that compounds of the invention, for instance SM875 or SM940, should be able to bind it, causing its stabilization and thus likely inducing its aggregation, since FI-PrP exposes to the solvent hydrophobic residues which instead are buried inside the natively folded protein core of PrP^{C}. Recombinant PrP aggregation has been evaluated by a detergent-insolubility assay. B. Representative Western blotting images showing the temperature-dependent effects on PrP aggregation exerted by compounds SM875 and SM940 Temperature increase was as follow: lanes 1,5, T=25° C.; lanes 2,6, T=37° C.; lanes 3,7, T=45° C.; lanes 4,8, T=55° C. C. Distribution graphs illustrating the results. Compounds SM875 and SM940 increased the amount of detergent-insoluble recombinant PrP in a temperature-dependent fashion. Conversely, no effects were detected in untreated controls, or when the protein was incubated with molecule SM935. These results indicate that compound SM875 and SM940 bind to a folding intermediate of PrP^{C} (i.e. FI-PrP). Statistical differences were estimated by one-way Anova, Dunnet post-hoc test. p values are: *<0.05, **<0.01.

FIG. 15: SM875 induces the activation of autophagy in a PrP^{C}-dependent fashion and its effects are inhibited by autophagy inhibitor Bafilomycin Al. A. First, HEK293 cells stably-transfected with mouse PrP^{C} or untransfected were treated with SM875 at different concentrations and then the levels of autophagy marker LC3II were evaluated by Western blotting. Representative pictures (above) and corresponding quantification (bar graph below) show that SM875 induces a large increase of LC3II in PrP^{C}-expressing cells (HEK293 WT-PrP) and a much smaller increase in untrasfected cells. However, it has to be noted that untransfected cells endogenously express low but still detectable levels of PrP^{C}, which may possibly account for the small increase in LC3III detected in these cells. Data were normalized on the effect of trehalose (TRE), a known autophagy inducer, which as expected increased LC3-II levels in both PrP^{C}-expressing and untransfected cells. B. Next, ZR-75 breast cancer cells endogenously expressing human PrP^{C} were treated with raising concentrations of SM875, in presence or absence of Bafilomycin A1 (BAF), a well-known inhibitor of lysosomal degradation. Representative pictures (above) and corresponding quantification (bar graph below) show that BAF almost completely prevents SM875-induced decrease of PrP^{C} levels. Collectively, these data indicate that SM875 promotes the post-translational degradation of PrP^{C} through the lysosome-dependent autophagy pathway. Statistical differences were estimated by one-way Anova, Dunnet post-hoc test. p values are: *<0.05, **<0.01, ***<0.001.

FIG. 16: SM875 inhibits prion replication in mouse fibroblasts. A. The schematic illustrates the experimental layout, using as an example the effect of Fe(III)-TMPyP, a previously described anti-prion compound (Massignan et al. SciRep 2016). B. Cells were incubated with raising concentrations of SM875 (indicated) or with TMPyP (10 μM) and the level of PK-resistant PrP was quantified by Western blotting. Results showed that, similarly to positive control TMPyP, SM875 inhibits prion levels in a dose-dependent fashion. Statistical differences were estimated by one-way Anova, Dunnet post-hoc test. p values are: *<0.05, **<0.01, ***<0.001.

FIG. 17: results of the effect on the expression of PrP of compounds according to the invention tested by western blotting in HEK293 cells stably expressing mouse PrP. Arrows compare the results of inhibitory concentration at 25% (IC₂₅) as compared to SM875.

FIG. 18: results of the effect on the expression of PrP of compounds according to the invention tested by western blotting in HEK293 cells stably expressing mouse PrP. Arrows compare the results of IC₅₀ as compared to SM875.

FIG. 19: A-F. Quantification of the PrP-lowering effects for indicated compounds (name, chemical formula, molecular weight and structure are reported) at different concentrations, as assayed by western blotting, plotted as percentage of the vehicle (Vhc) control. (**p<0.01 and ***p<0.005, by one-way ANOVA test).

FIG. 20: A-E. Quantification of the PrP-lowering effects for indicated compounds (name, chemical formula, molecular weight and structure are reported) at different concentrations, as assayed by western blotting, plotted as percentage of the vehicle (Vhc) control. (***p<0.005 by one-way ANOVA test).

FIG. 21: A-C. Quantification of the PrP-lowering effects for indicated compounds (name, chemical formula, molecular weight and structure are reported) at different concentrations, as assayed by western blotting, plotted as percentage of the vehicle (Vhc) control. (*p<0.05 by one-way ANOVA test).

FIG. 22: A-E. Quantification of the PrP-lowering effects for indicated compounds (name, chemical formula, molecular weight and structure are reported) at different concentrations, as assayed by western blotting, plotted as percentage of the vehicle (Vhc) control. (*p<0.05 by one-way ANOVA test).

FIG. 23: A-E. Quantification of the PrP-lowering effects for indicated compounds (name, chemical formula, molecular weight and structure are reported) at different concentrations, as assayed by western blotting, plotted as percentage of the vehicle (Vhc) control. (*p<0.05, **p<0.01 and ***p<0.005, by one-way ANOVA test).

FIG. 24: A-D. Quantification of the PrP-lowering effects for indicated compounds (name, chemical formula, molecular weight and structure are reported) at different concentrations, as assayed by western blotting, plotted as percentage of the vehicle (Vhc) control. (*p<0.05, **p<0.01 and ***p<0.005, by one-way ANOVA test).

FIG. 25: A-B. Quantification of the PrP-lowering effects for indicated compounds (name, chemical formula, molecular weight and structure are reported) at different concentrations, as assayed by western blotting, plotted as percentage of the vehicle (Vhc) control.

FIG. 26: A-B. Results on the PrP inhibition effects of the SM875 enantiomers (the stereocenter is indicated with asterisk). Quantification of the PrP-lowering effects is shown at different concentrations, as assayed by western blotting, and plotted as percentage of the vehicle (Vhc) control. (**p<0.01 and ***p<0.005, by one-way ANOVA test).

FIG. 27: Results of the dose response analysis on compound GC6 at six different concentrations in HEK293 cells and ZR-75 human breast cancer cells.

Quantification (B-D) of western blotting images (A-C) reporting the PrP-lowering effects for indicated compounds (name, chemical formula, molecular weight and structure are reported) at different concentrations, was plotted as percentage of the vehicle (Vhc) control. (*p<0.05, **p<0.01 and ***p<0.005, by one-way ANOVA test).

FIG. 28: Synthesis of SM875 analogues.

FIG. 29: Synthesis of methyl-derivatives.

FIG. 30: Synthesis of DG3.

### DETAILED DESCRIPTION OF THE INVENTION

### Definitions

Within the meaning of the present invention, a disease or pathology is a condition resulting from a pathophysiological response to external or internal factors.

A disorder is a disruption to the normal or regular functions in the body or a part of the body as a result of a disease.

A syndrome is a term that refers to a disease or a disorder that has more than one identifying feature or symptom.

Within the present disclosure, the terms disease, disorder, pathology are used interchangeably and are to be intended in the broadest meaning of disease.

As used in the present invention, the term “treatment” refers to the use of a compound for therapeutic purposes, which include decrease or prevention of disease symptoms, slower progression of said symptoms, abrogation of symptoms, as well as total or partial abrogation of the cause of the disease.

Within the present invention, a neurodegenerative disease is a disease resulting from neurodegenerative processes, e.g. the progressive loss of structure or function of neurons, including neuronal death.

Neurodegenerative diseases include but are not limited to, Parkinson's disease, Alzheimer's disease, prion disease, amyotrophic lateral sclerosis.

Neuroinflammatory disorders are conditions where immune responses damage components of the nervous system.

A demyelinating disease is disease of the nervous system in which the myelin sheath of neurons is damaged.

As defined herein and in WO 2020/021493, the step of modelling a sequence in time of a protein folding pathway is carried out by means of computer simulations based on the Molecular Mechanics (MM) or Quantum-Mechanics Molecular Mechanics (QM-MM) approaches. Said computer simulations are based on Ratchet-and-pawl molecular dynamics, and/or a Bias Functional computation approach, and/or by means of a Self-Consistent Path Sampling computation approach.

More details on exemplary algorithms that can be effectively used to carry out the above mentioned step of modelling time evolution of a protein folding pathway can be found in the scientific papers “S. Orioli, S. a Beccara, and P. Faccioli, J. Chem. Phys. 147, 064108 (2017)”; “C. Camilloni, R. A. Broglia, and G. Tiana, J. Chem. Phys. 134, 045105 (2011)”; “S. a Beccara, L. Fant, and P. Faccioli, Phys. Rev. Lett. 114, 098103 (2015)”.

Within the context of the present invention, “folding pathway” describes the transition from an unfolded protein to its native fold over the course of time, i.e., how a chain of amino acids reaches its thermodynamically stable state. Within the context of the “protein folding” and “folding pathways”, mentioned in the present description, is intended within the endogenous protein synthesis, and not related to as denaturing or renaturing processes or to conformers or “short lived conformations”.

“Druggability” is the ability of a protein, or any “conformer” of a protein, to allow binding of a drug (e.g., a small molecule, any other organic compound, a peptide or an antibody), thus causing potential therapeutic benefits for patients. A “conformer” is each alternative conformation of the same polypeptide. It reflects the conformational isomerism of polypeptides and the statistical character of the thermodynamic states of macromolecules.

In the present description, consistently with a terminology commonly used in the field of pharmaceutical research, the term “pocket” indicates a spatial region of the protein tertiary structure suitable for binding a small molecule.

In particular, the concept of druggable pockets refers to a specific binding site of a disease-linked protein target capable to bind drug-like molecules thus obtaining a modulation of the protein biological function.

When the binding site is not known from a 3D structure (e.g., ligand-protein complex) or from other experimental data (e.g., drug resistance mutations), computational methods can be employed to suggest likely locations.

Accordingly, several properties and/or parameters and/or global pocket descriptors (and respective exemplary values) may be used to characterize binding sites/pockets.

For example, a suitable druggable pocket may be characterized by a root-mean-square-deviation (RMSD) larger than a root-mean-square-deviation threshold from the pocket present in the native state.

In a non-limiting example, said root-mean-square-deviation threshold is equal to 2 Ångström (Å) or greater than 2 Ångström (Å).

According to the present invention, the pocket is defined in terms of pocket parameters, which are instrumental to predict its druggability by means of comparison of said pocket parameters with respective thresholds.

Said pocket parameters comprise dimensional parameters, and/or form parameters, and/or position parameters, and/or ratio of hydrophobic to hydrophilic character.

In particular, dimensional pocket parameters may comprise volume of the pocket and/or depth of the pocket and/or the enclosure and exposure of the pocket. The exposure and enclosure properties provide a different measure of how open is the site to solvent.

In an implementation example, the pocket volume threshold is at least 300 Å³.

In an implementation example, the pocket exposure threshold is less than 0.5 and the pocket enclosure threshold is at least 0.7.

In an implementation example, the pocket depth threshold is at least 10 Å.

Scoring parameters for protein hot-spot identification comprise “SiteScore”, and/or “Dscore”, and/or “DrugScore” and or pocket balance.

These parameters are known in the field of modelling and characterization of protein and protein intermediates, and are based on a mix of values, related to different properties, suitable to evaluate the “druggability” of a protein native state or a protein folding intermediate.

In fact, such scoring parameters derive from known evaluation software packages.

For example, SiteMap (Halgren TA (2009) “Identifying and characterizing binding sites and assessing druggability”, J. Chem. Inf. Model 49: 377-389) predicts a site score (SiteScore) and druggabilty score (DScore) through a linear combination of only three single descriptors: the size of the binding pocket, its enclosure, and a penality for its hydrophilicity.

Another example is DoGSiteScorer (A. Volkamer, D. Kuhn, T. Grombacher, F. Rippmann, M. Rarey, “Combining global and local measures for structure-based druggability predictions” J. Chem. Inf. Model. 2012,52,360-37), which also generates a druggability score (DrugScore) which range from zero to one.

The selection is based on the comparison of the scoring parameters with respective thresholds.

Within the present invention, the scoring parameters have been defined as follows:

the pocket SiteScore threshold is 0.8.

the pocket DScore threshold is 0.9.

the pocket DrugScore threshold is 0.5.

the pocket balance threshold is 1.0.

Whilst criteria for the identification and selection of the folding intermediate and the druggable pocket as listed above, are based on a comparison of parameters and/or values related to the properties chosen with respective thresholds, the person skilled in the art can understand that exemplary values and respective threshold may be subject to change according to the type of protein or other requirements, without affecting the definition of druggable pocket.

The Pharmacological Protein Inactivation by Folded Intermediate Targeting (PPI-FIT methodology)

The method for identifying target protein folding intermediates suitable to be tested as targets for drug discovery procedures disclosed in WO 2020/021493 is applied in the present invention to the design and selection of compounds against the most kinetically and thermodynamically relevant folding intermediate of the prion protein, in order to stabilize such intermediate and inhibit its transition to the native form. In a cellular environment, said stabilized folding intermediate is recognized by the protein folding quality control machinery as an improperly folded polypeptide and sent to degradation.

Thus, the present inventors selected therapeutically relevant compounds capable of post-translationally decreasing the expression of the prion protein with therapeutic benefits in prion diseases, and possibly other disorders, for instance neurodegenerative disorders, linked to the toxicity-transducing activity of this protein.

As a first step, the present inventors reconstructed the entire sequence of events underlying the folding pathway of the prion protein at atomistic level of resolution.

This was done by means of all-atom molecular simulations based on the Bias Functional (BF) approach, as detailed in Methods.

According to this approach, a first a set of fully denatured protein conformations is obtained by means of molecular dynamics (MD) simulations performed at large temperature, starting from the protein crystal native structure.

Next, from each of the initial unfolded configurations many independent folding pathways are generated by means of a specific algorithm called ratchet-and-pawl molecular dynamics (rMD), which contains a biasing force to promote the rate of folding transitions.

Finally, the least-biased trajectory (LBT) in the set of rMD folding trajectories generated starting from the same unfolded condition was selected by computing the scoring functional defined Eq.(4) in Methods.

To identify the folding intermediates, all configurations visited by rMD trajectories are plotted in the plane defined by the instantaneous Root-Mean-Square Deviation (RMSD) to the native structure and the fraction of native contacts.

The folding intermediate is identified with a high density region in the frequency histogram on this plane, separated from the high density associated with the protein native state.

Configurations in the folding intermediate are then structurally grouped using standard clustering algorithms.

Finally, protein targets were identified from conformers in the said clusters, selected from LBTs and satisfying the PPI-FIT criteria.

Then, the present inventors employed in-silico modeling and virtual drug screening techniques to identify small ligands specific for the PrP^{C} folding intermediate identified herein. Potential binding sites unique in the structure of the folding intermediate and not present in the native conformation were identified on the surface of the PrP intermediate, taking into account typical properties of druggable pockets, such as volume, depth, enclosure/exposure and hydrophobicity, as shown in table 1.

TABLE 1

| Descriptor | Threshold | FI-PrP poket |
|---|---|---|
| SiteScore | ≥0.8 | 0.94 |
| DScore | ≥0.9 | 0.94 |
| Exposure | ≤0.5 | 0.47 |
| Enclosure | ≥0.7 | 0.75 |
| Balance | ≥1 | 1.58 |
| Volume | ≥300 Å³ | 313.54 |
| Depth | ≥10 Å | 13.59 |
| SimpleScore | ≥0.5 | 0.52 |
| DrugScore | ≥0.5 | 0.53 |

A solvent-accessible druggable pocket was selected between the displaced helix 1 and helix 3, defined by 14, non-continuous residues (152, 153, 156, 157, 158, 187, 196, 197, 198, 202, 203, 205, 206, 209 with reference to the PDB 1QLX sequence). The conformation of this pocket was the target of a virtual screening campaign performed by using BioSolveIT software and employing the Asinex Gold & Platinum collection of small molecules (˜3.2×105 commercially available compounds). Positive hits were filtered by different chemoinformatics tools to predict pan-assay interference (e.g. the selection of Pan-assay interference compounds (PAINS) that react nonspecifically with numerous biological targets rather than specifically affecting one desired target), and properties such as pharmacodynamics, physicochemical and ADME (adsorption, distribution, metabolism and excretion) properties. Compounds were clustered based on their chemical scaffold and a ranking list of candidate hits was generated.

Following biological validation as described in the examples herein, four chemical scaffolds for therapeutic molecules and several active compounds were selected.

Accordingly, a first object of the invention is represented by chemical scaffolds capable of inducing the degradation of the cellular prion protein, said scaffolds being identified with the Pharmacological Protein Inactivation by the Folded Intermediate Targeting (PPI-FIT) methodology.

According to the above detailed methodology, the scaffolds of the invention have surprisingly shown to bind in silico a specific pocket corresponding to a binding site in the folding intermediate of the PrP protein, which is absent in its native conformation.

According to a first exemplary embodiment, compounds identified according to the present invention are characterized by the following scaffold of general Formula I (scaffold I):

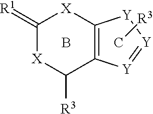

wherein:

ring B may be partially saturated, for instance a di-hydropyridinone ring when R¹ is 0 and/or when one or both X is N, or unsaturated, e.g. a pyridinone ring when

### R¹ is O;

R¹ may be O or S;

each atom on ring B may be independently a C or N atom;

X may be independently selected from —C— or —O— or —N(R⁴)—, wherein R⁴ may be —H, or —C₁₋₄ linear or cyclic alkyl;

ring C is a 5- or 6-membered aromatic ring wherein each atom may be —C— or —N—, e.g. Y may independently be —C(R³) or —N(R³)— or —N═;

R³ is selected from —H, C₁₋₄ linear or cyclic alkyl group, alkoxyl or aryloxyl, ring A or Z-ring A;

preferably, ring C has one or two heteroatoms;

Z may be —CH₂— or —O— or —N(H)— or —S(O₂)— or —S(O)—;

ring A may be an aromatic or heteroaromatic 5- or 6-membered ring, preferably a phenyl ring; ring A may be substituted at any position with one or more group R² independently selected from: —H, —F, —Cl, —Br, —I, —CH₃, —CH₂CH₃, —CH(CH₃)₂, —C(CH₃)₃, —CHF₂, —CF₃, —OH, —OCH₃, —OCH₂CH₃, —OCF₃, —OCH (CH₃)₂, —CN, —NO₂, —NH₂, —SH.

According to the present disclosure, when one or more stereocenters form within the above molecular structure, then the resulting enantiomers are encompassed within the purposes of the present invention.

For the purposes of the present invention, a preferred general formula I is the scaffold of formula I.1:

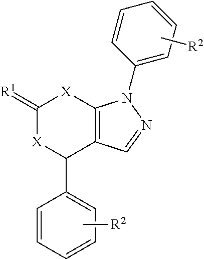

wherein the above definitions apply.

In particular, within general formula I.1: each atom on ring B may be independently a C or N atom, X may be independently selected from —C— or —O— or N(R⁴), R⁴ may be —H, or —C₁₋₄ linear or cyclic alkyl, R¹ may be 0 or S, R² may be independently selected from: —H, —F, —Cl, —Br, —I, —CH₃, —CH₂CH₃, —CH(CH₃)₂, —C(CH₃)₃, —CHF₂, —CF₃, —OH, —OCH₃, —OCH₂CH₃, —OCF₃, —OCH (CH₃)₂, —CN, —NO₂, —NH₂, —SH, wherein one or two or three R² may be present on each one of Ring A or Ring D.

According to preferred embodiments of the present invention, exemplary compounds according to general formula I are:

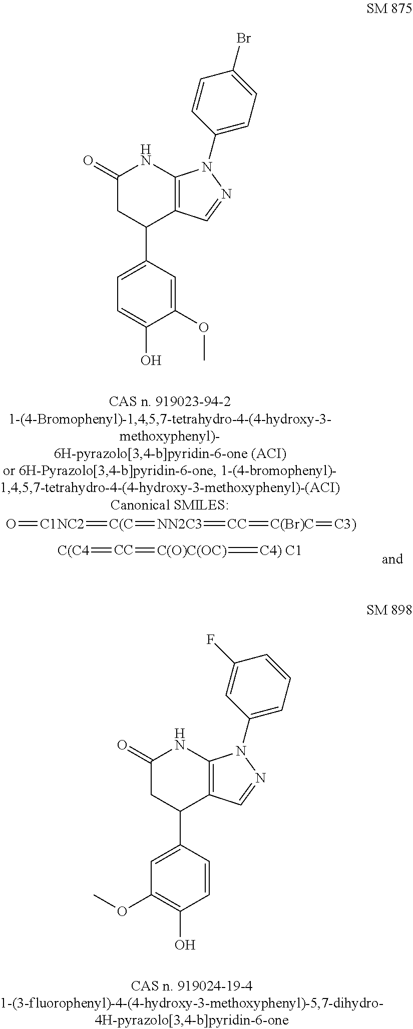

As per a particular aspect of the present invention, compound SM 875 comprises one stereocenter, so that the S and the R enantiomers form:

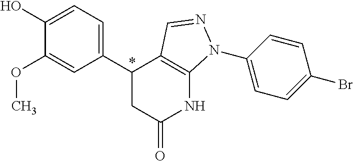 [SMILES: COc1cc(C2CC(=O)Nc3c2cnn3-c2ccc(Br)cc2)ccc1O]

Both enantiomers have been tested according to the experimental section reported below.

Particularly preferred in the enantiomeric form E2 having R configuration.

According to a second exemplary embodiment, compounds identified according to the present invention are characterized by the following scaffold of general formula II (scaffold II):

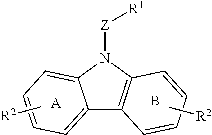

wherein:

each atom on each ring may be independently a —C— or —N— atom;

Z may be —H, —CH₂—, —S(O₂)—, —S(O)—,

R¹ may be absent (when Z is —H) or may be a group independently selected from: H, C₁₋₄ linear, branched, cyclic alkyl or alkoxyl or aryloxyl optionally substituted with one or more of: —H, —F, —Cl, —Br, —I, —CH₃, —CH₂CH₃, —CH (CH₃)₂, —C(CH₃)₃, —CHF₂, —CF₃, —OH, —OCH₃, —OCH₂CH₃, —OCF₃, —OCH (CH₃)₂, —CN, —NO₂, —NH₂, —S, a tetrahydrofuran ring or a tetrahydropyran ring wherein each atom of the ring may independently be substituted with —H or —OH, an aromatic or heteroaromatic 5- or 6-membered ring substituted at any position with one or more group R² independently selected from: —H, —F, —Cl, —Br, —I, —CH₃, —CH₂CH₃, —CH(CH₃)₂, —C(CH₃)₃, —CHF₂, —CF₃, —OH, —OCH₃, —OCH₂CH₃, —OCF₃, —OCH (CH₃)₂, —CN, —NO₂, —NH₂, —SH;

ring A and ring B are each independently an aromatic or heteroaromatic 5- or 6-membered ring, preferably a phenyl ring; ring A and ring B may be independently substituted at any position with one or more group R² independently selected from: —H, —F, —Cl, —Br, —I, —CH₃, —CH₂CH₃, —CH(CH₃)₂, —C(CH₃)₃, —CHF₂, —CF₃, —OH, —OCH₃, —OCH₂CH₃, —OCF₃, —OCH (CH₃)₂, —CN, —NO₂, —NH₂, —SH.

According to preferred embodiments of the present invention, an exemplary compound according to general formula II is:

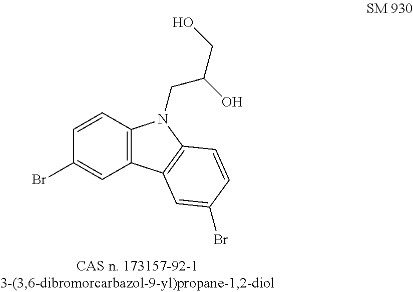

According to a third exemplary embodiment, compounds identified according to the present invention are characterized by the following scaffold of general formula III (scaffold III):

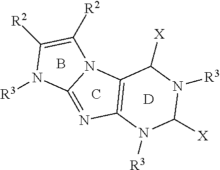

wherein:

each atom on each aromatic ring may be independently a C or N atom;

ring B and ring C are fused imidazole rings,

ring D is a pyrimidine ring, optionally ring D may be a cytosine or uracyl ring;

R² are each independently —H, —OH, —CH₂OH, —CH₃, —CH₂CH₃, halogen selected in the group comprising: —F, —Cl, —Br, —I, —CF₃, —OCH₃;

X may be independently: ═O, —NH₂, ═S;

R³ is independently selected from —H, C₁₋₄ linear, branched or cyclic alkyl, ring A or Z-ring A;

Z may be —CH₂ or —S(O₂)— or —S(O)—, ring A may be an aromatic or heteroaromatic 5- or 6-membered ring, preferably a phenyl ring; ring A may be substituted at any position with one or more group R¹ independently selected from: —H, —F, —Cl, —Br, —I, —CH₃, —CH₂CH₃, —CH(CH₃)₂, —C(CH₃)₃, —CHF₂, —CF₃, —OH, —OCH₃, —OCH₂CH₃, —OCF₃, —OCH (CH₃)₂, —CN, —NO₂, —NH₂, —SH.

For the purposes of the present invention, a preferred general formula III is scaffold of formula III.1:

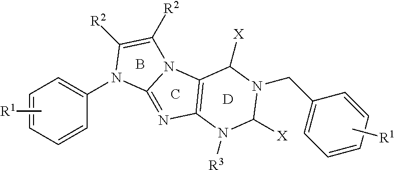

wherein the above definitions apply.

According to a preferred embodiment, an exemplary compound according to general formula III is:

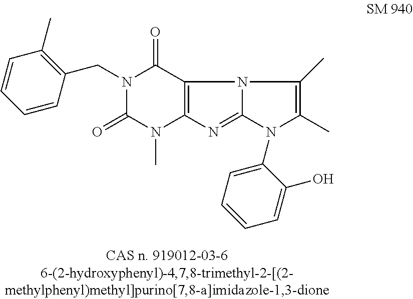

According to a fourth exemplary embodiment, compounds identified according to the present invention are characterized by a scaffold of general formula IV (scaffold IV):

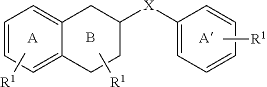

wherein:

each atom on each ring may be independently a C or N atom;

ring A and A′ are each independently an aromatic or heteroaromatic 5- or 6-membered ring, preferably a phenyl ring;

ring A, A′ and B may be substituted at any position with one or more group R¹ independently selected from: —H, —F, —Cl, —Br, —I, —CH₃, —CH₂CH₃, —CH (CH₃)₂, —C(CH₃)₃, —CHF₂, —CF₃, —OH, —OCH₃, —OCH₂CH₃, —OCF₃, —OCH (CH₃)₂, —CN, —NO₂, —NH₂, —SH;

ring B is an aromatic or a non-aromatic ring wherein each atom may be independently one or more C or N or O or S or S(O₂) atom and preferably is a piperidine ring;

X is —CH₂, —O—, —S—, —N(H), —C(O)—, —C(S)—, —C(H)=C (H)—, —S(O₂), —S(O).

For the purposes of the present invention, a preferred general formula IV is the following general formula IV.1:

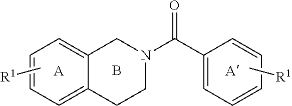

wherein the above definitions apply.

According to preferred embodiments of the present invention, an exemplary compound according to general formula I is:

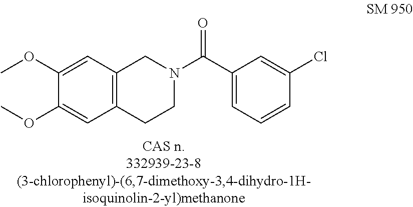

As per specific embodiments of the present invention, preferred compounds identified with the Pharmacological Protein Inactivation by the Folded Intermediate Targeting (PPI-FIT) methodology according to the above have one of the following formula:

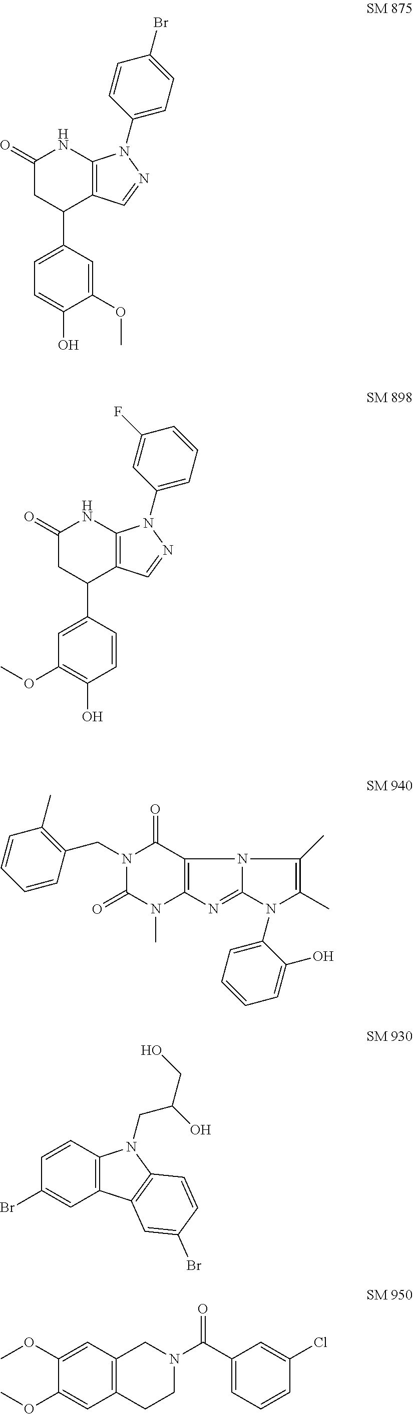

In particular, the preferred compounds of the invention are those above referred as SM875, SM898 and SM940.

Within the meaning of the present invention, a preferred general formula I.1 is the scaffold of formula I.2:

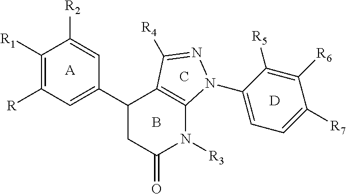

wherein Ring A and Ring D may independently be aromatic or heteroaromatic rings, preferably wherein the heteroatom is represented by N.

Most preferably, each Ring A and/or Ring D has one, two or three heteroatoms, preferably one, two, three N atoms, most preferably one or two N atoms.

In an embodiment, Ring A is selected from:

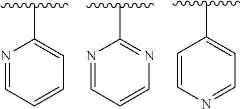

In an embodiment, Ring D is selected from:

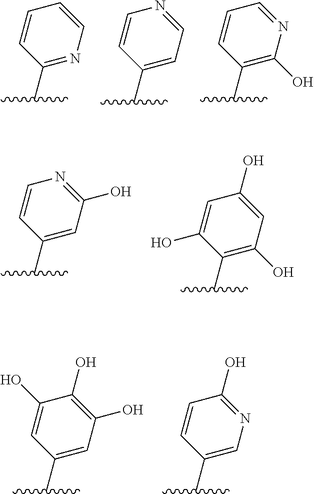

R, R₁ and R₂ may independently be H, hydroxyl (e.g., —OH), C₁₋₃ alkoxyl group (for instance represented but not limited to —OCH₃, —OCH₂CH₃, —OCH(CH₃)₂), phenoxyl (e.g., —OPh), benzyloxyl (e.g. —OCH₂Ph).

In an embodiment, R and R₁ may form a 1,4-dioxane ring. In a further embodiment, R₁ is phenoxyl (e.g., —OPh) or benzyloxyl (e.g., —OCH₂Ph).
R₁ may also be one of

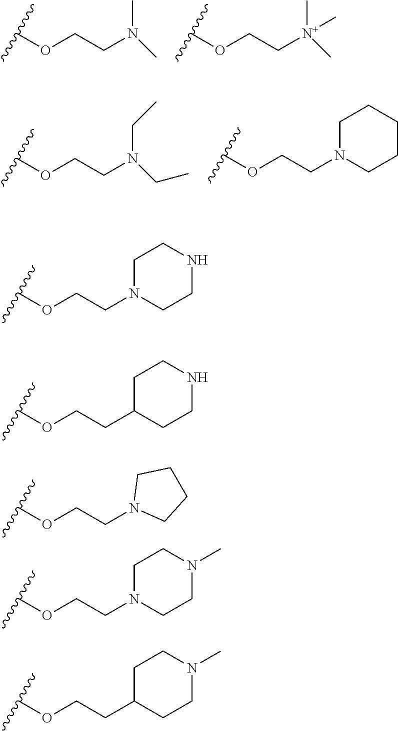

R₃ may be H, C₁-3 alkyl (preferably, methyl); in one embodiment, R₃ is H; in a further embodiment, R₃ is methyl.
R₄ may be H, C₁-3 alkyl (preferably, methyl); in one embodiment R₄ is H, in a further embodiment R₄ is methyl.
R₅, R₆, R₇ may independently be: H, hydroxyl (—OH) halide, (e.g., —F, —Br, —Cl, —I), methyl (e.g., —CH₃), triflouromethyl (e.g., —CF₃), hydroxyl (e.g., —OH).
Ring D may have one, two or three substituents; in a preferred embodiment, R₅ and R₆ are H; in a particularly preferred embodiment, when R₅ and R₆ are H, R₇ is I or Br.

According to a further embodiment of the present invention, a preferred structure within formula I.1 and 1.2 is formula 1.3 wherein Ring A and Ring D are aromatic rings;

R, R₁ and R₂ may be H, hydroxyl (e.g., —OH), C_{1-c3} alkoxyl group (e.g., —OCH₃, —OCH₂CH₃, —OCH (CH₃)₂), phenoxyl (e.g., —OPh) or benzyloxyl (e.g., —OCH₂Ph).
In an embodiment, R and R₁ may form a 1,4-dioxane ring. R₃ may be H, C₁₋₃ alkyl (preferably methyl); in one embodiment, R₃ is H, in a further embodiment R₃ is methyl.
R₄ may be H, C₁₋₃ alkyl (preferably methyl); in one embodiment R₄ is H, in a further embodiment R₄ is methyl.
R₅, R₆, R₇ may independently be H, halide, (e.g., —F, —Br, —Cl, —I), methyl (e.g., —CH₃), triflouromethyl (e.g., —CF₃). Ring D may have one, two or three substituents; in a preferred embodiment, R₅ and R₆ are H; in a particularly preferred embodiment, when R₅ and R₆ are H, R₇ is I or Br.

According to a further embodiment of the present invention, a preferred structure within formula I.1, I.2, I.3 is Formula I.4 wherein

Ring A and Ring D are aromatic rings and preferably are represented each by a phenyl ring;
R, R₁ and R₂ may be H, hydroxyl (e.g., —OH), C₁₋₃ alkoxyl group (e.g., —OCH₃, —OCH₂CH₃, —OCH (CH₃) 2),
R₃ may be H, C₁-3 alkyl (preferably, methyl),
R₄ may be C₁₋₃ alkyl (preferably, methyl),
R₅, R₆, R₇ may independently be H, halide (e.g. —F, —Br, —Cl, —I), methyl (e.g., —CH₃), triflouromethyl (e.g., —CF₃).
Ring D may have one, two or three substituents; in a preferred embodiment, R₅ and R₆ are H; in a particularly preferred embodiment, when R₅ and R₆ are H, R₇ is I or Br.

According to a further embodiment of the present invention, a preferred structure within formula I.1, 1.2, 1.3, 1.4, is Formula 1.5 wherein: Ring A and Ring D are aromatic rings and preferably are represented each by a phenyl ring,

when R is —OH, R₁ is —OCH₃,
when R is —OCH₃, R₁ is —OH,
when R is —H, —OPh or —OCH₂Ph,
when R is —OCH₂CH₃, R₁ is —O—CH₃,
R and R₁ form a 1,4-dioxane ring,
R₄ is H, or methyl,

### R₅ is —H,

### R₆ is —H,

R₇ is —H, —Br, —F, —I, trifluoromethyl (—CF₃), —Cl.

According to a particular aspect of the invention, within the compounds of the disclosed formula 1.2, 1.3, 1.4 and 1.5:

optionally, when R₁ is —OH, R or R₂ is not —OCH₂CH₃; when R or R₂ is —OCH₂CH₃, R₁ is not —OH;
optionally, when R₁ is —OH and R or R₂ is —OCH₂CH₃, R₅ is not —CH₂CH₃.
Optionally, when R or R₂ is-OH, R₁ is not —OCH₃;
optionally, when R₁ is —OCH₃, R or R₂ is not —OH; optionally, when R₁ is —OCH₃, R or R₂ is —OH, R₇ is not —CH₂CH₃.
Optionally, when R and R₂ are —OCH₃, R₁ is not —OH;
optionally, when R₁ is —OH, R₂ and R₃ are not —OCH₃; optionally, when R and R₂ are —OCH₃, R₁ is-OH, R₆ is not —F.
Optionally, R₅ is not —CH₂CH₃.
Optionally, R-7 is not —CH₂CH₃.

### Optionally, R₆ is not —F.

As per an aspect of the present invention, the disclosed compounds are not:

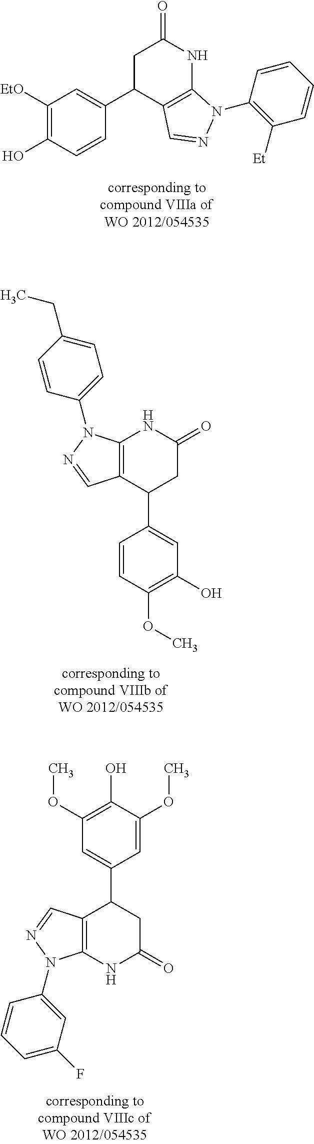 [SMILES: CCOc1cc(C2CC(=O)Nc3c2cnn3-c2ccccc2CC)ccc1O ; CCc1ccc(-n2ncc3c2NC(=O)CC3c2ccc(OC)c(O)c2)cc1 ; COc1cc(C2CC(=O)Nc3c2cnn3-c2cccc(F)c2)cc(OC)c1O]

According to particular aspects of the invention, there are disclosed the following compounds:

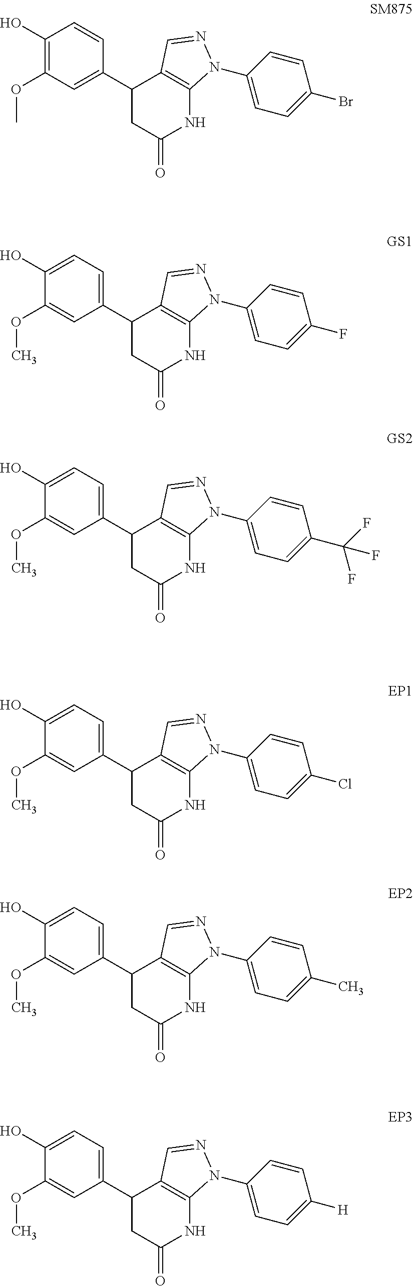 [SMILES: COc1cc(C2CC(=O)Nc3c2cnn3-c2ccc(Br)cc2)ccc1O ; COc1cc(C2CC(=O)Nc3c2cnn3-c2ccc(F)cc2)ccc1O ; COc1cc(C2CC(=O)Nc3c2cnn3-c2ccc(C(F)(F)F)cc2)ccc1O ; COc1cc(C2CC(=O)Nc3c2cnn3-c2ccc(Cl)cc2)ccc1O ; COc1cc(C2CC(=O)Nc3c2cnn3-c2ccc(C)cc2)ccc1O ; COc1cc(C2CC(=O)Nc3c2cnn3-c2ccccc2)ccc1O]

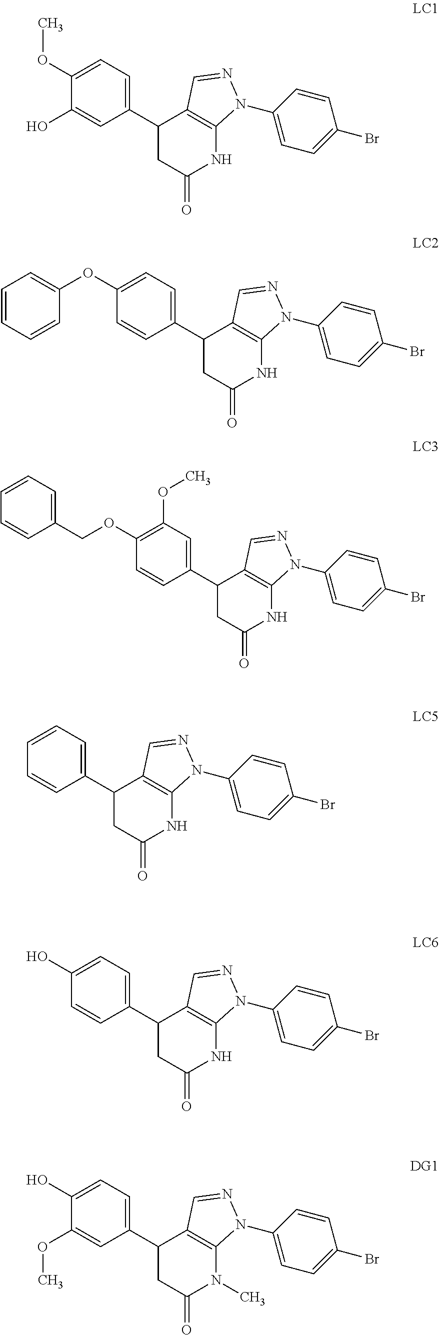 [SMILES: COc1ccc(C2CC(=O)Nc3c2cnn3-c2ccc(Br)cc2)cc1O ; O=C1CC(c2ccc(Oc3ccccc3)cc2)c2cnn(-c3ccc(Br)cc3)c2N1 ; COc1cc(C2CC(=O)Nc3c2cnn3-c2ccc(Br)cc2)ccc1OCc1ccccc1 ; O=C1CC(c2ccccc2)c2cnn(-c3ccc(Br)cc3)c2N1 ; O=C1CC(c2ccc(O)cc2)c2cnn(-c3ccc(Br)cc3)c2N1 ; COc1cc(C2CC(=O)N(C)c3c2cnn3-c2ccc(Br)cc2)ccc1O]

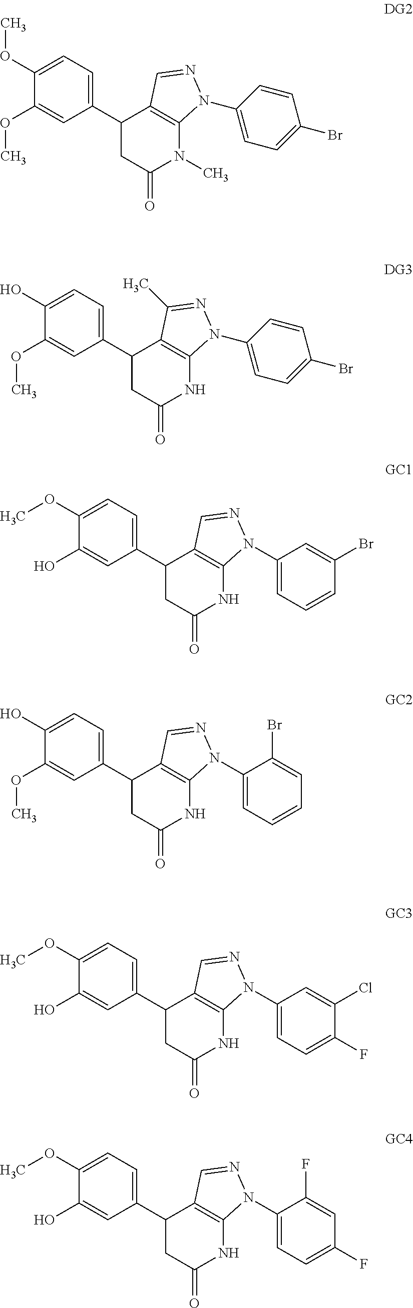 [SMILES: COc1ccc(C2CC(=O)N(C)c3c2cnn3-c2ccc(Br)cc2)cc1OC ; COc1cc(C2CC(=O)Nc3c2c(C)nn3-c2ccc(Br)cc2)ccc1O ; COc1ccc(C2CC(=O)Nc3c2cnn3-c2cccc(Br)c2)cc1O ; COc1cc(C2CC(=O)Nc3c2cnn3-c2ccccc2Br)ccc1O ; COc1ccc(C2CC(=O)Nc3c2cnn3-c2ccc(F)c(Cl)c2)cc1O ; COc1ccc(C2CC(=O)Nc3c2cnn3-c2ccc(F)cc2F)cc1O]

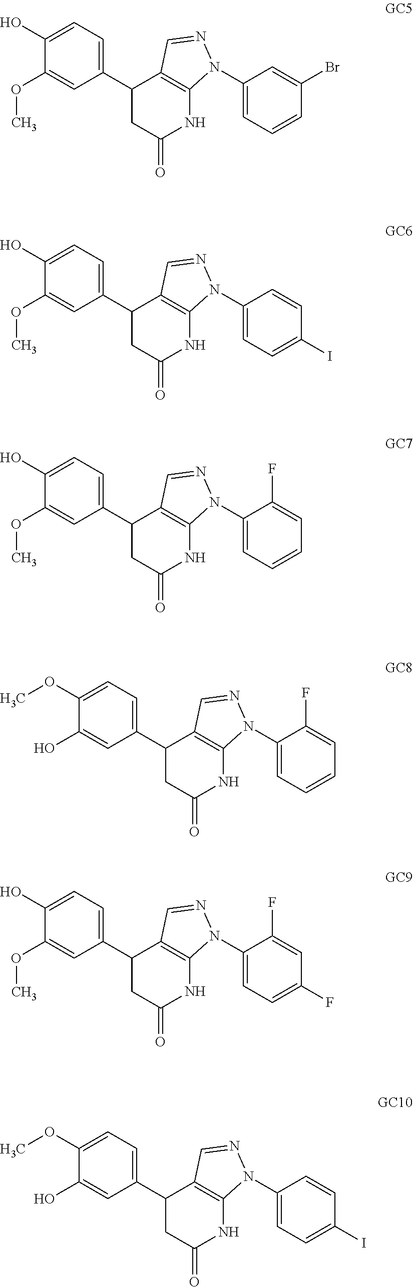 [SMILES: COc1cc(C2CC(=O)Nc3c2cnn3-c2cccc(Br)c2)ccc1O ; COc1cc(C2CC(=O)Nc3c2cnn3-c2ccc(I)cc2)ccc1O ; COc1cc(C2CC(=O)Nc3c2cnn3-c2ccccc2F)ccc1O ; COc1ccc(C2CC(=O)Nc3c2cnn3-c2ccccc2F)cc1O ; COc1cc(C2CC(=O)Nc3c2cnn3-c2ccc(F)cc2F)ccc1O ; COc1ccc(C2CC(=O)Nc3c2cnn3-c2ccc(I)cc2)cc1O]

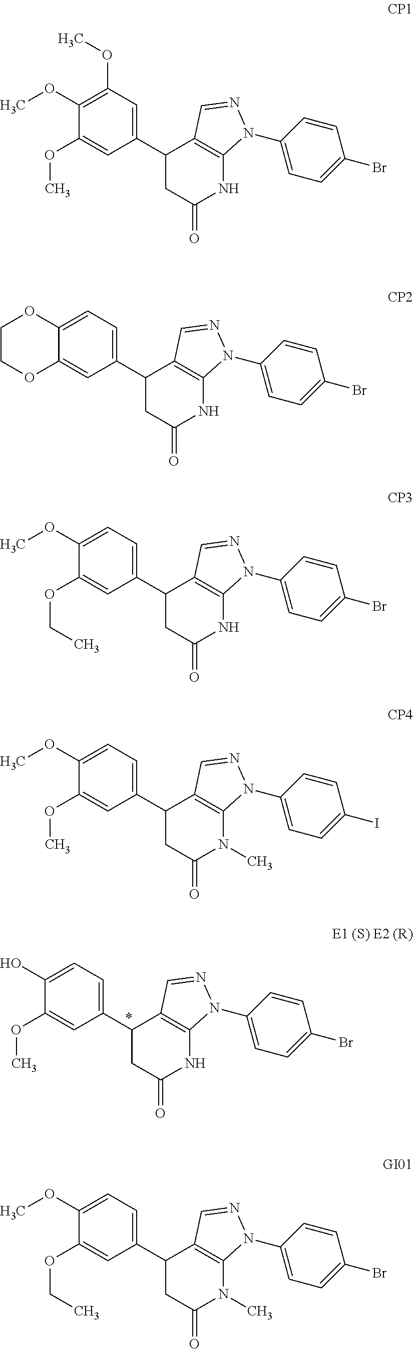 [SMILES: COc1cc(C2CC(=O)Nc3c2cnn3-c2ccc(Br)cc2)cc(OC)c1OC ; O=C1CC(c2ccc3c(c2)OCCO3)c2cnn(-c3ccc(Br)cc3)c2N1 ; CCOc1cc(C2CC(=O)Nc3c2cnn3-c2ccc(Br)cc2)ccc1OC ; COc1ccc(C2CC(=O)N(C)c3c2cnn3-c2ccc(I)cc2)cc1OC ; COc1cc(C2CC(=O)Nc3c2cnn3-c2ccc(Br)cc2)ccc1O ; CCOc1cc(C2CC(=O)N(C)c3c2cnn3-c2ccc(Br)cc2)ccc1OC]

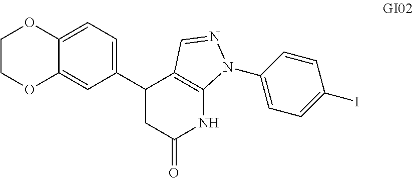 [SMILES: O=C1CC(c2ccc3c(c2)OCCO3)c2cnn(-c3ccc(I)cc3)c2N1]

Particularly preferred compounds of the present invention are GS2, LC1, DG3, GC6, LC2, LC3, LC5, LC6, CP2, CP3, GIO2.

For the purposes of the present invention, the disclosed compounds may comprise one or more stereocenters, so that enantiomers form.

All said enantiomers shall be considered within the present invention.

The compounds disclosed in the present invention are all commercially available and/or they can be prepared according to methods known in the art or according to the following examples.

In a second object, the present invention discloses pharmaceutical compositions comprising the invention compounds.

As per a particular aspect, a pharmaceutical composition may include also an enantiomeric form of the compounds of the invention.

In a third object, the invention discloses the compounds of general formula (I, II, III and IV as well as formula 1.1, 1.2, 1.3, 1.4 and 1.5, 111.1, IV.1), specific compounds reported above and their enantiomeric forms as well as the pharmaceutical compositions comprising them, for use as a medicament.

In particular, the invention compounds are disclosed for use as a medicament in the treatment of diseases or disorders related to the cellular prion protein (PrP^{C}) or interactors of PrP^{C} or toxic signaling pathways involving PrP^{C}, including neurodegenerative disorders, such as sporadic, inherited or acquired prion diseases, Alzheimer's disease, Parkinson's diseases and other α-synucleinopathies; neuroinflammatory disorders and demyelinating diseases, such as multiple sclerosis; cancer, in particular glioblastoma, gastric cancer, breast cancer, colon cancer.

In a preferred embodiment, compounds of general formula (I, II, III and IV as well as formula 1.1, 1.2, 1.3, 1.4 and 1.5, 111.1, IV.1), specific compounds reported above and their enantiomeric forms are disclosed for use as a medicament in the treatment of prion diseases, Alzheimer's disease, Parkinson's diseases, multiple sclerosis, glioblastoma, gastric cancer, breast cancer, colon cancer.

In particular, prion diseases include Creutzfeldt-Jakob disease (CJD), Gerstmann-Straussler-Scheinker (GSS) syndrome and fatal familial insomnia (FFI).

According to a further embodiment of the invention, the compounds of the invention are disclosed for use in the treatment of a disease, wherein the term “treatment” refers to therapeutic purposes, which include decrease or prevention of disease symptoms, slower progression of said symptoms, abrogation of symptoms, as well as total or partial abrogation of the cause of the disease.

In another embodiment, the discovered scaffolds are disclosed for the treatment of demyelinating disorders, including but not limited to multiple sclerosis.

According to a further embodiment, the compounds of the invention are disclosed for use in the treatment of the above diseases together with other pharmacological or non-pharmacological treatments.

In particular, a pharmacological synergy may be shown between the compounds of the invention and one or more other medicaments.

More in particular, said one or more medicaments may be against PrP^{C} or interactors of PrP^{C} or toxic signaling pathways involving PrP^{C} such as:
small molecules
antibodies,
Proteins or peptides
RNA based therapeutics
gene therapy or gene editing therapeutic approaches,
   against PrP^{C} or interactors of PrP^{C} or toxic signaling pathways involving PrP^{C}.

Therefore, the compounds of the invention are disclosed for medical use in combination with one or more medicaments against diseases or disorders related to the cellular prion protein (PrP^{C}) or interactors of PrP^{C} or toxic signaling pathways involving PrP^{C} including small molecules, antibodies, protein or peptides, RNA based therapeutics, gene therapy or gene editing therapeutic approaches.

In particular, said diseases or disorders related to the cellular prion protein (PrP^{C}) or interactors of PrP^{C} or toxic signaling pathways involving PrP^{C} comprise neurodegenerative disorders, such as sporadic, inherited or acquired prion diseases, Alzheimer's disease, Parkinson's diseases and other α-synucleinopathies; neuroinflammatory disorders and demyelinating diseases, such as multiple sclerosis; cancer, in particular glioblastoma, gastric cancer, breast cancer, colon cancer.

In a further object, it is disclosed a method for the treatment of disorders related to the cellular prion protein (PrP^{C}) or interactors of PrP^{C} or toxic signaling pathways involving PrP^{C} comprising the administration of a compound of the invention or of a pharmaceutical composition comprising a compound of the invention, to a patient in need thereof.

According to a further embodiment of the invention, there is disclosed a method comprising the administration of a compound of the invention for the treatment of a disease, wherein the term “treatment” refers to therapeutic purposes, which include decrease or prevention of disease symptoms, slower progression of said symptoms, abrogation of symptoms, as well as total or partial abrogation of the cause of the disease.

In particular, within the method of the invention, said administration may be initiated before the disease onset (particularly in the case of genetic disorders for which the disease-causing mutations have been assessed), immediately after the disorder has been diagnosed, or once other pharmaceutical treatments have proved to fail.

In another embodiment, the discovered scaffolds are disclosed in a method for the treatment of demyelinating disorders, including but not limited to multiple sclerosis.

Within the disclosed method, the compounds of the invention may be administered together with other pharmacological or non-pharmacological treatments.

In particular, a pharmacological synergy may be shown between the compounds of the invention and:
small molecules
antibodies,
protein or peptides,
RNA-based therapeutics
gene therapy or gene editing therapeutic approaches.

According to an aspect of the invention, said compounds may be against PrP^{C} or interactors of PrP^{C} or toxic signaling pathways involving PrP^{C}.

The compounds of the invention are disclosed in a method of treatment in combination with small molecules, antibodies, gene therapy or gene editing therapeutic approaches, optionally against diseases or disorders related to the cellular prion protein (PrP^{C}) or interactors of PrP^{C} or toxic signaling pathways involving PrP^{C}.

In particular, said diseases or disorders related to the cellular prion protein (PrP^{C}) or interactors of PrP^{C} or toxic signaling pathways involving PrP^{C} comprise neurodegenerative disorders, such as sporadic, inherited or acquired prion diseases, Alzheimer's disease, Parkinson's diseases and other α-synucleinopathies; neuroinflammatory disorders and demyelinating diseases, such as multiple sclerosis; cancer, in particular glioblastoma, gastric cancer, breast cancer, colon cancer.

Within the disclosed method, the compounds of the invention may therefore be administered together with other pharmacological or non-pharmacological treatments against neurodegenerative disorders, such as sporadic, inherited or acquired prion diseases, Alzheimer's disease, Parkinson's diseases and other α-synucleinopathies; neuroinflammatory disorders and demyelinating diseases, such as multiple sclerosis; cancer, in particular glioblastoma, gastric cancer, breast cancer, colon cancer.

In particular, a pharmacological synergy may be shown between the compounds of the invention and:
small molecules
antibodies,
protein or peptides,
RNA-based therapeutics
gene therapy or gene editing therapeutic approaches,
   against neurodegenerative disorders, such as sporadic, inherited or acquired prion diseases, Alzheimer's disease, Parkinson's diseases and other α-synucleinopathies; neuroinflammatory disorders and demyelinating diseases, such as multiple sclerosis; cancer, in particular glioblastoma, gastric cancer, breast cancer, colon cancer.

According to a further embodiment, there is disclosed the use of the compounds of the present invention for the manufacture of a medicament for the treatment of diseases or disorders related to the cellular prion protein (PrP^{C}) or interactors of PrP^{C} or toxic signaling pathways involving PrP^{C}.

In particular, said diseases or disorders are represented by neurodegenerative disorders, such as sporadic, inherited or acquired prion diseases, Alzheimer's disease, Parkinson's diseases and other α-synucleinopathies; neuroinflammatory disorders and demyelinating diseases, such as multiple sclerosis; cancer, in particular glioblastoma, gastric cancer, breast cancer, colon cancer.

The present invention will be further disclosed with reference to the following experimental section.

### Methods

### Bias Functional Methods for Folding Pathways Simulations.

General Features and Software. The BF method is a three-step procedure that enables the simulation of protein folding pathways at atomistic level of resolution and consists in: (i) generation of denatured condition by thermal unfolding, (ii) productions of folding trajectories starting from the unfolded states and (iii) scoring of the folding trajectories based on a variational principle. The software to perform these simulations relies on the MD engine of Gromacs 4.6.5 patched with the plugin for collective variable analysis Plumed 2.0.2.
Generation of Denatured Conditions. Unfolded conformations are generated by thermal unfolding starting from the native structure. This is achieved by performing independent 3-5 ns trajectories of molecular dynamics at high temperature (800 K) in the NVT ensemble. For each trajectory, a single denatured conformation is extracted.
Generation of Folding Pathways. For each denatured conformation, a set of folding trajectories are generated by employing the rMD algorithm. In this scheme, the folding progress is described as a function of a reaction coordinate, defined as z(X):

z(X)=Σ_{|i-j|>35}^{N}[Cij(X)−Cij(X)^{Native}]² Equation 1

Where Cij(X) is the contact map of the instantaneous system configuration and Cij(X)Native is the contact map in the reference native state. The reference native state is obtained by energy minimizing the experimental structure retrieved from the protein data bank. The Cij(X) entries of z(X) interpolate smoothly between 0 and 1 according to the following function:

$${C}_{ij} ( X ) = \{ \begin{matrix}\frac{1 - {( {r}_{ij} / {r}_{0} )}^{6}}{1 - {( {r}_{ij} / {r}_{0} )}^{10}} & if {r}_{ij} \leq {r}_{c} \\ 6 / 10 & if {r}_{ij} = {r}_{0} \\ 0 & if {r}_{ij} > {r}_{c}\end{matrix} \qquad Equation 2$$

Where rij is the Euclidean distance between the ith and the jth atom, r0 is a typical distance defying residue contacts (set to 7.5 A) and rc is a cutoff distance (set to 12.3 A) beyond which the contact is set to 0. In rMD, the protein evolves according to plain-MD as long as the reaction spontaneously proceeds towards the native state (i.e. lowering the value of the z(x) coordinate). On the other hand, when the chain tries to backtrack along z(X), an external biasing force is introduced that redirects the dynamics towards the native state. The biasing force acting on a given atom, FirMD, is defined as:

$${F}_{i}^{rMD} = \{ \begin{matrix}- {k}_{r} {∇}_{i} z ( X ) \cdot \{ z ( X ) - {z}_{m} ( t ) \} & if z ( X ) > {z}_{m} \\ 0 & if z ( X ) \leq {z}_{m}\end{matrix} \qquad Equation 3$$

where zm(t) indicates the smallest value of the reaction coordinate z(X) up to time t and kr is a coupling constant.
Selection of the Least Biased Trajectory. For each set of trajectories starting from the same initial condition, the folding pathway with the highest probability to realize in absence of external biasing force is selected. This scheme is applied by first defining a folding threshold: a trajectory is considered to have reached the folded state if its root mean squared deviation of atomic positions (RMSD) compared to the native target structure is ≤4 Å. Then, the trajectories successfully reaching the native state are scored by their computed bias functional T, defined as:

$$T = {\sum }_{i = 1}^{N} \frac{1}{{m}_{i} {γ}_{i}} {\int }_{0}^{t} d τ {\vert {F}_{i}^{rMD} ( X , τ ) \vert}^{2} \qquad Equation 4$$

Where t is the trajectory folding time, mi and yi are the mass and the friction coefficient of the ith atom and FirMD is the force acting on it. The folding trajectory minimizing the bias functional for each set is referred to as Least Biased (LB) trajectory.

### Computational Analyses of the Cellular Prion Protein.

Structure and Topology of the C-terminal Domain of the Cellular Prion Protein (PrP). The native structure of the C-terminal domain of human PrP was retrieved from PDB 1QLX, the structure spans from residue 125 to 228 and contain the structured globular domain of PrP.
Protein topology was generated in Gromacs 4.6.5 using Amber99SB-ILDN force field in TIP3P water.
Folding Simulations of PrP. The native structure of the C-terminal domain of human PrP (PDB 1QLX) was positioned in a dodecahedral box with 40 Å minimum distance from the walls. The box was filled with spc216 water molecules and neutralized with 3 Na+ ions. The system was energy minimized using the steepest descent algorithm. NVT equilibration was then performed for 500 ps at 800 K using the V-rescale thermostat with positional restraints on heavy atoms. Restraints were then removed and 9 independent 3 ns of plain MD were performed in the NVT ensemble at 800 K, yielding 9 denatured conformation. Each initial condition was repositioned in a dodecahedral box with 15 Å minimum distance from the walls, energy minimized using the steepest descent algorithm and then equilibrated first in the NVT ensemble (using the Nosé-Hoover thermostat at 350 K, τT=1 ps) and then in the NPT ensemble (using the Nos6-Hoover thermostat at 350 K, τT=1 ps, and the Parrinello-Rahman barostat at 1 bar, τP=2 ps). For each initial condition 20 trajectories were generated by employing the rMD algorithm in the NPT ensemble (350 K, 1 bar). Each trajectory consists in 1.5·10⁶ rMD steps generated with a leap-frog integrator with time-step of 2 fs. Frames were saved every 5·10² steps. The ratchet constant kr was set to 5·10⁻⁴ kJ/mol. Non-bonded interactions were treaded as follow: Van-der-Waals and Coulomb cutoff was set to 16 A, while Particle Mesh Ewald was employed for long-range electrostatics. For each set of trajectories, the bias functional scheme was applied with an additional filtering on the secondary structure content for folding definition. In particular, trajectories reaching a final conformation with less than 85% of average secondary structure content compared to the NMR structure were not considered in the ranking. Analysis of Trajectories. RMSD was computed using Gromacs while the fraction of native contacts (Q) was computed using VMD 1.9.2. A lower-bound approximation of the energy landscape G(Q, RMSD) was generated by plotting the negative logarithm of the 2D probability distribution of the collective variables Q and RMSD, obtained from the 180 rMD trajectories (115×115 bins). Protein conformations belonging to the LB trajectories and spanning over the energetic wells of interest (G<3.7 kBT) were sampled. Conformations belonging to the intermediate state were clustered by using a k-mean clustering in R-Studio employing the following metrics for defining a distance between two structures:

D(X_{A},X_{B})=\sqrt{Σ_{|i-j|}^{N}[Cᵢⱼ(X_{A})−Cᵢⱼ(X_{B})]²} Equation 5

Where D(XA, XB) is the distance metrics between two protein conformations, Cij (XA) and Cij (XB) are the contact map entries of the conformations A and B respectively (defined in equation 2). The appropriate number of cluster (k=3) was selected using the elbow method. The representative configuration of each cluster was selected by calculating the average contact map of the cluster conformations and then extracting the structure minimizing the distance D(XA, XB) between itself and the average contact map. Data were represented using the Matplotlib library in python, the 2D [Q, RMSD] energy plot was smoothed with a Gaussian kernel. Images of the protein conformations were produced using UCSF Chimera.

### Computer-Aided Drug Discovery Analyses.

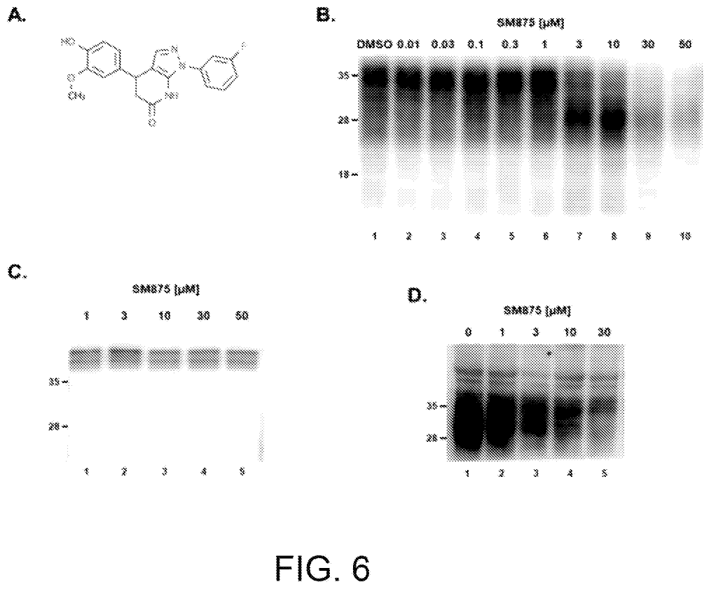

Consensus Approach for Druggable Ligand Binding Site Identification. A consensus approach relying on SiteMap (Schrödinger Release 2017-4: SiteMap, Schrödinger, LLC, New York, N.Y., 2017; Halgren, T., J. Chem. Inf. Model., 2009, 49, 377-389) and DoGSiteScorer (A. Volkamer, J. Chem. Inf. Model. 2012, 52 (2), pp 360-372) analysis has been applied to find and evaluate druggable binding pocket. The default parameters were used for both tools. Considering the innovative character of the target herein reported (i.e. a folding intermediate structure), less stringent thresholds were thus selected in the search of druggable pockets to be explored in virtual screening: volume≥300 Å3; depth≥10 Å; balance≥1.0; exposure≤0.5; enclosure≥0.70; SiteScore≥0.8; DScore≥0.90; DrugScore≥0.5; SimpleScore≥0.5 (Supp. Table 2 and 3).
Identification of a Druggable Pocket in the PrP Folding Intermediate. The folding intermediate was prepared with the Schroedinger's Protein Preparation Wizard. During the preparation, the hydrogen bonding networks were optimized through an exhaustive sampling of hydroxyl and thiol groups. The N- and C-terminal residues were capped with ACE and NMA groups, respectively. Then, hydrogen atoms and protein side chains were energy-minimized using the OPLS3 force field. The obtained structure was (i) solvated by TIP3P water molecules in a cubic simulation box of 12.5 Å distant from the protein in every direction, (ii) neutralized by addition of three Na+ ions, and (iii) equilibrated for 100 ps of MD simulation (NPT ensemble) at 300 K using the Langevin thermostat. In such a simulation, the relative positions of the Ca atoms were kept fixed (force constant 1 Kcal/mol), in order to exclusively sample the arrangement of the side chains. MD simulations were performed using the OPLS3 force field in Desmond 5.0 software (Schrödinger Release 2017-4: Desmond Molecular Dynamics System, D. E. Shaw Research, New York, N.Y., 2017) and run for 50 ns. Recording interval was set to 50 ps, allowing the collection of 1001 frames. The trajectory was clustered using the “Desmond trajectory clustering” tool in Maestro (Maestro-Desmond Interoperability Tools, Schrödinger, New York, N.Y., 2017) based on the RMSD of residues 152, 153, 156, 157, 158, 187, 196, 197, 198, 202, 203, 205, 206 and 209 (i.e. the residues composing the interested site). A hierarchical clustering was performed to obtain 10 clusters of the explored site. The centroid of each cluster was then selected as representative structure and subjected to in silico ligand binding site prediction and druggability assessment by using the above-mentioned consensus methods involving DogSiteScorer and SiteMap analysis. Virtual Chemical Library Preparation. The Asinex Gold & Platinum Library was downloaded from the Asinex webpage (˜3,2×10⁵ commercially available compounds, www.asinex.com). A first round of ligand preparation was performed in LigPrep (Schrödinger Release 2017-4: LigPrep, Schrödinger, LLC, New York, N.Y., 2017). In this step, the different tautomeric forms for undefined chiral centers were created. By contrast, for specified chirality, only the specified enantiomer was retained. Subsequently, the compounds were imported within SeeSAR (SeeSAR Version 5.6, BioSolveIT GmbH, Sankt Augustin, Germany, 2016). A final library of ˜4.3×105 docking clients was thus obtained.
Identification of In Silico Hits Through Virtual Screening. The virtual screening workflow was developed by using the KNIME analytic platform (Berthold, Michael R. AcM SIGKDD explorations Newsletter 11, no. 1 (2009): 26-31) and the BioSolveIT KNIME nodes. Specifically, the workflow was organized as follows: (i) the “Prepare Receptor with LeadIT” node was used for protein preparation and docking parameters definition in LeadIT (LeadIT version 2.2.0; BioSolveIT GmbH, Sankt Augustin, Germany, 2017, www.biosolveit.de/LeadIT). The binding site was defined on the basis of the residues composing the identified druggable pocket (Supp Table 3). The residue protonation states, as well as the tautomeric forms, were automatically assessed in LeadIT using the ProToss method, that generates the most probable hydrogen positions on the basis of an optimal hydrogen bonding network using an empirical scoring function; (ii) the “Compute LeadIT Docking” node was selected to perform the docking simulations of the ˜4.3×10⁵ docking clients by using the FlexX algorithm (Rarey M, et al. J Mol Biol. 1996 Aug 23;261(3):470-89). Ten poses for each ligand were produced; (iii) the “Assess Affinity with HYDE in SeeSAR” node generated refined binding free energy (i.e. AG) and estimated HYDE affinity (KiHYDE) for each ligand pose using the HYDE rescoring function (N. Schneider, et al. J. Comput. Aided. Mol. Des., 27 (2013), pp. 15-29); (iv) for each ligand, the pose with the lowest KiHYDE was extracted. Only compounds with a predicted KiHYDE range below 5 uM were retained for the following steps; (v) the rescored poses were filtered based on physicochemical and ADME filters using the Optibrium models integrated in SeeSAR (Optibrium 2018, www.optibrium.com/stardrop). In particular the following filters were used: 2≤Log P≤5, where Log P is the calculated octanol/water partition coefficient; 1.7≤Log D≤5, where log D is the calculated octanol/water distribution coefficient; TPSA≤90; Log S≥1, where TPSA is the topological polar surface area; Log S7.4≥1, where Log S7.4 is the intrinsic aqueous solubility at pH of 7.4 (a Log S≥1 corresponds to intrinsic aqueous solubility of greater than 10 μM); HIA=+, where HIA is the classification for human intestinal absorption (predicts a classification of ‘+’ for compounds which are ≥30% absorbed and ‘−’ for compounds which are <30% absorbed); 300≤MW≤500, where MW is molecular weight; number of rotable bonds≤3; PgP category =−, where PgP category is the classification of P-glycoprotein transport (the compound must belong to the ‘-’ category to avoid the active efflux); number of hydrogen bond donors ≤3; number of stereocenters ≤1. In addition, molecules potentially acting as pan-assay interference compounds were discharged. This approach produced a list of 275 virtual hits, that were first submitted to a diversity-based selection. For each compound, a binary fingerprint was derived by means of the canvasFPgen utility provided by Schrödinger (Fingerprint type: MolPrint2D; precision:XP) (Schrödinger Release 2017-4: Canvas, Schrödinger, LLC, New York, N.Y., 2017). Using the created fingerprint, the 10 most different compounds (i.e. ASN 03578729, ASN 15755504, ASN 16356773, ASN 17325626, ASN 19380113, BAS 00312802, BAS 00340795, BAS 00382671, BAS 01058340, BAS 01849776) were extrapolated by applying the canvasLibOpt Schrodinger utility (Schrödinger Release 2017-4: Canvas, Schrödinger, LLC, New York, N.Y., 2017). In addition, visual inspection guided the selection of promising ligands based on the predicted binding mode and the interactions established with the identified binding pocket. In total, 30 molecules were selected, 8 from the diverse selection and 22 after visual inspection (Supp FIG. 6 and Supp Table 4). Indeed, even though 10 diverse compounds were originally chosen, BAS 00340795 was not in stock and ASN 03578729 was later replaced by its close analogue ASN 05397475, selected by 3D visualization and endowed with a better predicted affinity.

### Chemical Synthesis of SM875.

Reagents and Instrumentation. The reagents (Sigma Aldrich) and solvents (Merck) were used without purification. The reaction yields were not optimized and calculated after chromatographic purification. Thin layer chromatography (TLC) was carried out on Merck Kieselgel 60 PF254 with visualization by UV light at 254 nm. Microwave-assisted reactions were carried out using a mono-mode CEM Discover reactor in a sealed vessel. Preparative thin layer chromatography (PLC) on 20×20 cm Merck Kieselgel 60 F254 0.5 mm plates. HPLC purification was performed by a Merck Hitachi L-6200 apparatus, equipped with a diode array detector Jasco UVIDEC 100V and a LiChrospher reversed phase RP18 column, in isocratic conditions with eluent acetonitrile/water 1:1, flow 5 mL min⁻¹, detection at 254 nm. IR spectrum of the final product was recorded by using a FT-IR Tensor 27 Bruker spectrometer equipped with Attenuated Transmitter Reflection (ATR) device at 1 cm⁻¹ resolution in the absorption region Δν 4000÷1000 cm⁻¹. A thin solid layer was obtained by the evaporation of the chloroform solution in the sample. The instrument was purged with a constant dry nitrogen flow. Spectra processing was made using Opus software packaging. NMR spectra were recorded on a Bruker-Avance 400 spectrometer by using a 5 mm BBI probe ¹H at 400 MHz and ¹³C at 100 MHz in CDCl₃ relative to the solvent residual signals δ_{H} 7.25 and δ_{C} 77.00 ppm, J values in Hz. Structural assignments are confirmed by heteronuclear multiple bond correlation (HMBC) experiment. ESI-MS spectra were taken with a Bruker Esquire-LC mass spectrometer equipped with an electrospray ion source, by injecting the samples into the source from a methanol solution MS conditions: source temp. 300° C., nebulizing gas N₂, 4 L·min⁻¹, cone voltage 32 V, scan range m/z 100-900. Fragmentation experiments were carried out using helium to collisionally activate the selected primary ions. LC-ESI-MS spectrum was acquired using a C-18 Kinetex 5 μm column, eluting with acetonitrile/water 70:30, flow 1 mL·min⁻¹ using ESI source as detector in positive ion mode. High-resolution ESI-MS measurement for the final product, including tandem MS² fragmentation experiments, were obtained by direct infusion using an Orbitrap Fusion Tribrid mass spectrometer.
Cell cultures and treatments.
Cell lines used in the present invention have been cultured in Dulbecco's Minimal Essential Medium (DMEM, Gibco, #11960-044), 10% heat-inactivated fetal bovine serum (Δ56-FBS), Penicillin/Streptomycin (Pen/Strep, Corning #20-002-Cl), non-essential amino acids (NEAA, Gibco, #11140-035) and L-Glutamine (Gibco, #25030-024), unless specified differently. HEK293 and N2a cells were obtained from ATCC (ATCC CRL-1573 and CCL-131, respectively). We used a subclone (A23) of HEK293 stably expressing a mouse wild-type PrP or an EGFP-PrP construct, both already described and characterized previously (Stincardini et al. 2017). L929 mouse fibroblasts and inducible RK13 cells were kindly provided by Ina Vorberg (DZEN, Bonn, Germany) (Vorberg et al. 2004) and Didier Villette (INRA, Toulouse, France) (Arellano-Anaya et al. 2017), respectively. Human cancer cell lines (H₃₅₈, ZR-75, A549, H₄₆₀, MCF7, H₁₂₉₉, SKBR3 and T47D), all belonging to the NCI collection of human cancer cell lines, were kindly provided Valentina Bonetto (Mario Negri Institute, Milan, Italy). Cells were passaged in T25 flasks or 100 mm Petri dishes in media containing 200 pg/mL f Hygromycin or 500 pg/ml of G418 and split every 3-4 days. Every cell line employed in this study has not been passaged more than 20 times from the original stock. Compounds used in the experiments were resuspended at 30 or 50 mM in DMSO, and diluted to make a 1000× stock solution, which was then used for serial dilutions. A 1 μl aliquot of each compound dilution point was then added to cells plated in 1 mL of media with no selection antibiotics.
For pulse experiments, inducible RK13 cells were seeded on 24-well plates at a confluence of 50%. After 24 h cells were treated with doxyciclin (0.01 mg/ml) or vehicle (DMSO), in the presence or absence of Brefeldin 1A (10 μM) or SM875 (10 μM). At the end of each time-point (2, 4, 8 and 24 h) cells were washed with PBS and then lysed in lysis buffer. For chase experiments, RK13 cells were seeded on 24-well plates at a confluence of 30%. After 24 h cells were treated with Doxyciclin (0.01 mg/ml) for 24 h. The medium containing doxyciclin was then removed and cells kept in fresh medium for 4 h before adding SM875 (10 μM). After 5, 19 and 24 h of incubation cells wells were washed with PBS and lysed.

### Plasmids.

Cloning strategies used to generate cDNAs encoding for WT or EGFP-tagged PrP have been described previously (Ivanova et al. 2001). The EGFP-PrP construct contains a monomerized version of EGFP inserted after codon 34 of mouse PrP. The identity of all constructs was confirmed by sequencing the entire coding region. All constructs were cloned into the pcDNA3.1(+)/hygro expression plasmid (Invitrogen). The Strep-FLAG pcDNA3.1(+)/G418 NEGR-1 was kindly provided by Giovanni Piccoli (University of Trento, Italy) (Pischedda et al. 2014). All plasmids were transfected using Lipofectamine 2000 (Life Technologies), following manufacturer's instructions.

### Western Blotting & Antibodies.

Samples were lysed in lysis buffer (Tris 10 mM, pH 7.4, 0.5% NP-40, 0.5% TX-100, 150 mM NaCl plus complete EDTA-free Protease Inhibitor Cocktail Tablets, Roche, #11697498001), diluted 2:1 in 4× Laemmli sample buffer (Bio-Rad) containing 100 mM Dithiothreitol (CAS No. 3483-12-3, SigmaAldrich), boiled 8 min at 95° C. and loaded on SDS-PAGE, using 12% acrylamide pre-cast gels (Bio-Rad) and then transferred to polyvinylidene fluoride (PVDF) membranes (Thermo Fisher Scientific). Membranes were blocked for 20 min in 5% (w/v) non-fat dry milk in Tris-buffered saline containing 0.01% Tween-20 (TBS-T). Blots were probed with anti-PrP antibody D18 (kindly provided by D. Burton, The Scripps Research Institute, La Jolla, Calif.) or 6D11 (1:5,000) in BSA 3% in TBS-T overnight at 4° C., washed 3 times with TBS-T 10 min each, then probed with a 1:8,000 dilution of horseradish conjugated goat anti-human (Jackson Immunoresearch) or anti-mouse (Santa Cruz) IgG for 1 h at RT. After 2 washes with TBS-T and one with Milli-Q water, signals were revealed using the ECL Prime Western Blotting Detection Kit (GE Healthcare), and visualized with a ChemiDoc XRS Touch Imaging System (Bio-Rad).

### Dot Blotting.

Samples were spotted on PVDF membrane in a 96-well dot blot system. A 96-well dot blot apparatus (Schleicher & Schuell) was set up with a 0.45-μm-pore-size polyvinylidene difluoride (PVDF) membrane (Immobilon-P; Millipore), and each dot was rinsed with 500 μl of TBS. Under vacuum, cell lysates were added to the apparatus rinsed with 500 μL of TBS. The membrane was then blocked with 5% milk-0.05% Tween 20 (Sigma) in TBS (TBST-milk) for 30 min and probed with the anti-PrP antibody 6D11 (1:4,000) followed by goat anti-mouse IgG (Pierce). Signals were revealed using enhanced chemiluminescence (Luminata, Bio-Rad) and visualized by a ChemiDoc XRS Touch Imaging System (Bio-Rad).

### Real Time PCR.

Following treatments, cells were harvested from 24 wells plates and RNA was extracted using TRIzol (Invitrogen) or RNeasy Plus mini kit (Quiagen). A 800 ng aliquot per each sample was reverse transcribed using High Capacity cDNA Reverse Transcription kit (Applied Biosystems) according to the manufacturer's instructions. Quantitative RT-PCR was performed in a CFX96 Touch thermocycler (Bio-Rad) using PowerUp SYBR Green Master mix (Invitrogen) with a program of 40 cycles amplification. Mouse PrP set 1 and set 2 were respectively used to amplify endogenous and transgenic PrP (table below). Relative quantification was normalized to mouse or human HPRT (hypoxanthine-guanine phosphoribosyltransferase).

| | Forward sequence | Reverse sequence |
|---|---|---|
| Primer | 5′-3′ | 5′-3′ |
| Mouse HPRT | TCAGACCGCTTTTTGC | ATCGCTAATCACGACG |
| (endogenous) | CGCGA | CTGGGAC |
| Mouse PrP | GGACATCACCAAGACG | CGCCATGATGACTGAT |
| set 1 | AGGG | CCGA |
| (endogenous) | | |
| Mouse PrP | GCTGGCCCTCTTTGTG | GTTCCACCCTCCAGGC |
| set 2 | ACTA | TTTG |
| (exogenous) | | |
| Human HPRT | CAGCCCTGGCGTCGTG | TCACATCTCGAGCAAG |
| (endogenous) | ATTAGTGA | ACGTTCAGT |
| Human PrP | CCGAGGCAGAGCAGTC | CCAGGTCACTCCATGT |
| (endogenous) | ATTA | GGC |

### Inmunofluorescence and High Content Imaging.

Immunocytochemistry was performed on inducible RK13 cells treated for 2, 4, 8, or 24 h with doxycycline 0.01 (1×) or 0.1 (10×) mg/mL, in the presence or absence of SM875 (10 μM). Cells were seeded on CellCarrier-384 Ultra microplates (Perkin Elmer) at a concentration of 6,000 cells/well, grown for approximately 24 h, to obtain a semi-confluent layer (60%) and then treated with the compound. Cells were fixed for 12 min at RT by adding methanol-free paraformaldehyde (PFA, Thermo Fisher Scientific) to a final concentration of 4%. Wells were then washed three times with PBS, and permeabilization was performed by incubating cells for 1 min with PBS containing a final concentration of 0.1% Triton X-100. Wells were washed three times with PBS and cells were incubated with blocking solution (FBS 2% in PBS) for 1 h at RT. The anti-PrP primary antibody (D18) was diluted in the blocking solution and added to the wells to a final concentration of 1:400. After three washes with PBS, the secondary antibody (Alexa 488-conjugated goat anti-human IgG diluted 1:500 in blocking solution) was incubated for 1 h at RT. Hoechst 33342 (Thermo Fisher Scientific) diluted in 0.5 mM PBS was then added after two additional washes.
For high content imaging experiments, cells expressing EGFP-PrP were plated on CellCarrier-384 Ultra microplates (Perkin Elmer) at a concentration of 12,000 cells/well and grown for approximately 24 h, to obtain a semi-confluent layer (60%). SM875 was administered at a final concentration of 0.1, 0.3, 1, 3 or 10 μM, in two replicate wells. Vehicle (DMSO, volume equivalent) was used as a negative control. Cells were treated for 24 h and then fixed for 12 min at RT by adding methanol-free paraformaldehyde (Thermo Fisher Scientific) to a final concentration of 4%. Plates were then washed twice with PBS and counterstained with Hoechst 33342 (Thermo Fisher Scientific). The cell localization of EGFP-PrP and inducible PrP was monitored using an Operetta High-Content Imaging System (Perkin Elmer). Imaging was performed in a widefield mode using a 20X High NA objective (0.75). Five fields were acquired in each well over two channels (380-445 Excitation-Emission for Hoechst and 475-525 for EGFP and Alexa 488). Image analysis was performed using the Harmony software version 4.1 (Perkin Elmer).
Cell viability.
Cells were seeded on 24-well plates at approximately ˜60% confluence. Compounds at different concentrations or vehicle control (DMSO, volume equivalent) were added after 24 h, medium was replaced the second day, and then removed after a total of 48 treatment. Cells were incubated with 1 mg/mL of 3-(4,5-dimethylthiazol-2-yl)-2,5-diphenyltetrazolium bromide (MTT, Sigma Aldrich) in PBS for 15 min at 37° C. After carefully removing MTT, cells were resuspended in 500 μL DMSO, and cell viability values obtained by a plate spectrophotometer (BioTek Instruments, VT, USA), measuring absorbance at 570 nm.
Production of recombinant PrP. RecHuPrP23-231 was expressed by competent E. coli Rosetta (DE3) bacteria harboring pOPIN E expression vector containing the wild type 1109 bank vole Prnp gene (Erana et al. 2019). Bacteria from a glycerolate maintained at −80° C. were grown in a 250 ml Erlenmeyer flask containing 50 ml of LB broth overnight. The culture was then transferred to two 2 L Erlenmeyer flasks containing each 500 ml of minimal medium supplemented with 3 g/L glucose, 1 g/L NH₄Cl, 1M MgSO₄, 0.1 M CaCl₂), 10 mg/mL thiamine and 10 mg/mL biotin. When the culture reached an OD₆₀₀ of ˜0.9-1.2 AU, Isopropyl B-D-1-thiogalactopyranoside (IPTG) was added to induce expression of PrP overnight under the same temperature and agitation conditions. Bacteria were then pelleted, lysed, inclusion bodies collected by centrifugation, and solubilized in 20 mM Tris-Cl, 0.5 M NaCl, 6M Gnd/HCl, pH=8. Although the protein does not contain a His-tag, purification of the protein was performed with a histidine affinity column (HisTrap FF crude 5 ml, GE Healthcare Amersham) taking advantage of the natural His present in the octapeptide repeat region of PrP. After elution with buffer containing 20 mM Tris-HCl, 0.5 M NaCl, 500 mM imidazole and 2 M guanidine-HCl, pH=8, the quality and purity of protein batches was assessed by BlueSafe (NZYTech, Lisbon) staining after electrophoresis in SDS-PAGE gels. The protein was folded to the PrP^{C} conformation by dialysis against 20 mM NaAcetate buffer, pH=5. Aggregated material was removed by centrifugation. Correct folding was confirmed by CD and protein concentration, by measurement of absorbance at 280 nm. The protein was concentrated using Amicon centrifugal devices and the concentrated solution stored at −80° C. until used.
Dynamic mass redistribution.
The EnSight Multimode Plate Reader (Perkin Elmer) was used to carry out DMR analyses. Immobilization of full-length (residues 23-230) or mouse N-terminally truncated (111-230) recombinant PrP (15 μL/well of a 2.5 μM PrP solution in 10 mM sodium acetate buffer, pH 5) on label-free microplates (EnSpire-LFB high sensitivity microplates, Perkin Elmer) was obtained by amine-coupling chemistry. The interaction between Fe³⁺-TMPyP, SM875 and SM940 diluted to different concentrations (0.03-100 μM, eight 1:3 serial dilutions) in assay buffer (10 mM PO₄, pH 7.5, 2.4 mM KCl, 138 mM NaCl, 0.05% Tween-20) and PrP, was monitored after a 30 min incubation at RT. All the steps were executed by employing a Zephyr Compact Liquid Handling Workstation (Perkin Elmer). Data were obtained by normalizing each signal on the intra-well empty surface, and then by subtraction of the control wells. The Kaleido software (Perkin Elmer) was employed to acquire and process the data.
Temperature-dependent detergent insolubility assay.
An 800 μM stock solution of freshly purified mouse recombinant PrP (residues 111-231) was diluted 1:10 in Sodium Acetate (10 mM NaAc, pH 7) to obtain 80 μM aliquots. To avoid precipitation of recombinant PrP, aliquots were flash freeze in liquid nitrogen, stored at −80° C., and then kept on ice during their use. In order to carry out the assay, recombinant PrP was diluted to a final concentration of 0.5 μM in precipitation buffer (10 mM NaAc, 2% TX100, pH 7), split in 8 identical aliquots, and incubated for 1 h at different temperatures (25, 37, 45, and 55° C.), in the presence or absence of each molecule, or vehicle control (DMSO, volume equivalent). Each sample was then carefully loaded onto a double layer of sucrose (60% and 80%) prepared in precipitation buffer and deposited at the bottom of ultracentrifuge tubes. Samples were then subjected to ultracentrifugation at 100,000×g for 1 hour at 4° C. The obtained protein pellets were diluted in 2× LMSB and then analyzed by Western blotting.
Detection of prions in cells.
L929 fibroblasts were grown in culturing medium and passaged 5-7 times after infection with the RML prion strain (derived from corresponding prion-infected mice, courtesy of Dr. Roberto Chiesa, Mario Negri Institute, Milan). In order to test the anti-prion effects of compounds, cells were seeded in 24-well plates (day 1) at approximately 60% confluence, with different concentrations of each molecule, or vehicle control (DMSO, volume equivalent). Medium containing fresh compounds or vehicle was replaced on day 2, and cells were split (1:2) on day 3, avoiding the use of trypsin by pipetting directly onto the well surface. Cells were collected on day 4 in PBS and centrifuged at 3.500 rpm×3 min. The resulting pellets were then rapidly stored −80° C. To evaluate prion loads, cell pellets were resuspended in 20 μL of lysis buffer (Tris 10 mM, pH 7.4, 0.5% NP-40, 0.5% TX-100, 150 mM NaCl) and incubated for 10 min at 37° C. with 2.000 units/mL of DNase I (New England BioLabs). Half of the resulting sample was incubated with 10 pg/mL of PK (Sigma Aldrich) for 1 h at 37° C., while the other half was incubated in the same conditions in the absence of PK. Both PK-treated and untreated samples were then mixed 1:2 with 4× Laemmli sample buffer (Bio-Rad) containing DTT, boiled for 8 min at 95° C. and ran by SDS-PAGE.
Statistical analyses of biological data.
All the data were collected and analyzed blindly by two different operators. Statistical analyses, performed with the Prism software version 7.0 (GraphPad), included all the data points obtained, with the exception of experiments in which negative and/or positive controls did not give the expected outcome. No test for outliers was employed. The Kolmogorov-Smirnov normality test was applied (when possible, n≥5). Results were expressed as the mean±standard errors, unless specified. In some case, the dose-response experiments were fitted with a 4-parameter logistic (4 PL) non-linear regression model, and fitting was estimated by calculating the R². All the data were analyzed with the one-way ANOVA test, including an assessment of the normality of data, and corrected by the Dunnet post-hoc test. Probability (p) values <0.05 were considered as significant (*<0.05, **<0.01, ***<0.001).

### EXAMPLE 1

### Identification of a PrP folding intermediate

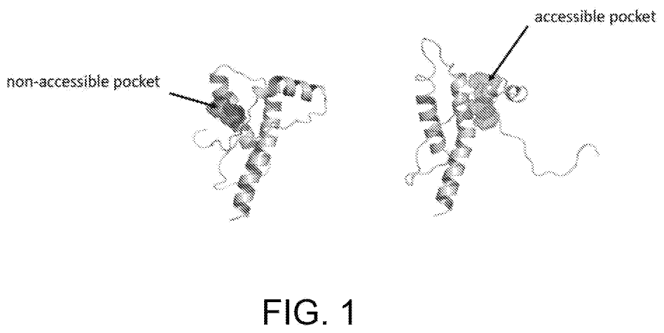

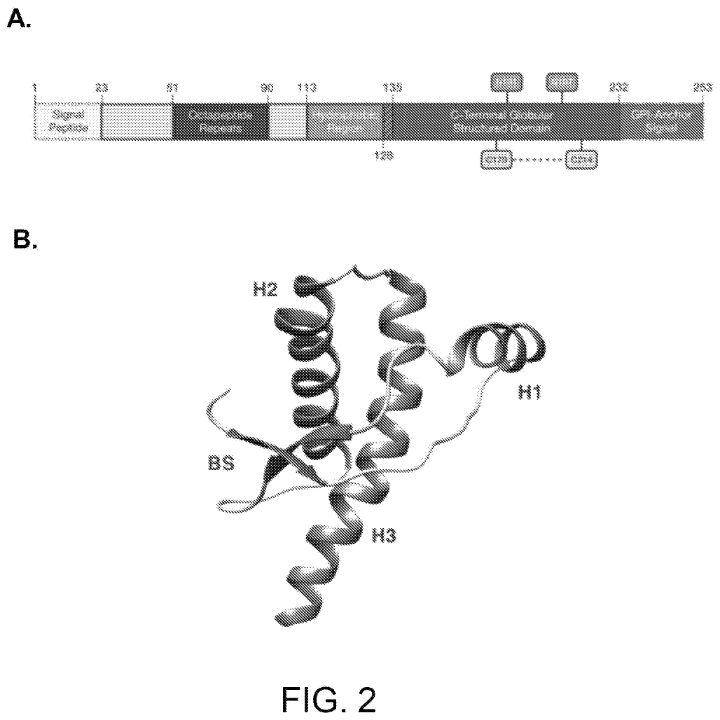

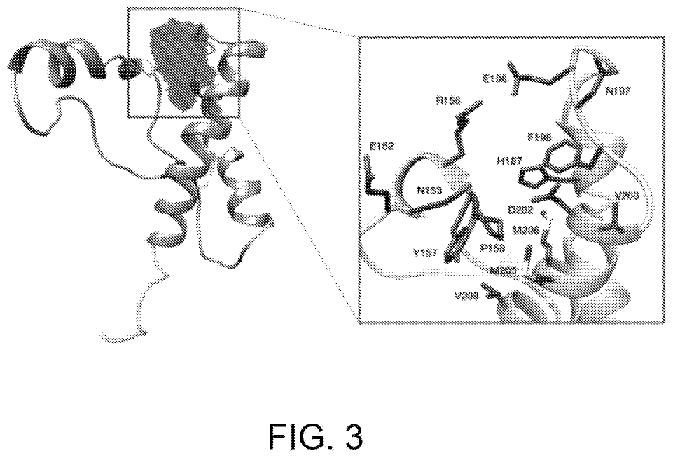

The PPI-FIT methodology was used to identify a druggable intermediate along the folding pathway of PrP^{C} (named FI-PrP, FIG. 1, right). To characterize the protein folding pathway and identify kinetically relevant intermediates the inventors relied on an enhanced path sampling technique (Bias Functional approach) using the AMBER ff99SB-ILDN force fieldin explicit solvent.
The prion protein consists of a flexible, N-terminal segment (residues 24-120 of the human sequence with reference to the PDB 1QLX sequence), and a structured, C-terminal domain (residues 121-230) comprising three α-helices and two short β-strands flanking helix 1. The N-terminal segment is natively unstructured and precludes its use for in-silico approaches. Thus, as a reference structure the globular domain of human PrP (residues 121-230, as shown in FIG. 2) was used. We generated 9 unfolded PrP conformations by thermal unfolding and used rMD to produce 20 folding trajectories for each conformation. The BF scheme was then employed to define the most statistically-significant folding pathway for each set of trajectories, leading to the 9 least biased folding pathways. Conformations residing within the observed energy wells in the bidimensional distribution were sampled from each least biased trajectory. The analyses revealed the existence of a semi-native, intermediate PrP folding state explored by all least biased pathways. Subsequent clustering analysis of the ensemble of conformers populating the intermediate state enabled the definition of an all-atom structure for the PrP folding intermediate (as shown in FIG. 3, left).

As compared to the PrP native conformation, this structure is characterized by a displaced helix-1 missing its docking contacts with helix 3, leaving exposed to the solvent several hydrophobic residues natively buried inside the protein core (e.g. Y157, M206, V209; FIG. 3, right).

### Virtual Identification of High Affinity Ligands for the PrP Folding Intermediate.

In-silico modeling and virtual drug screening techniques were employed to identify small ligands specific for the PrP folding intermediate selected. First, the inventors searched for potential binding sites unique in the structure of the intermediate and not present in the native conformation. The SiteMap and DoGSiteScorer software were used to scout the surface of the PrP intermediate, taking into account typical properties of druggable pockets, such as volume, depth, enclosure/exposure and hydrophobicity (as shown in Table 1). These analyses identified a solvent-accessible druggable pocket between the displaced helix 1 and helix 3, defined by 14, non-continuous residues (152, 153, 156, 157, 158, 187, 196, 197, 198, 202, 203, 205, 206, 209). The conformation of this pocket, refined by performing MD with positional restrain of backbone atoms, as shown in FIG. 3), was the target of a virtual screening campaign performed by using BioSolveIT software and employing the Asinex Gold & Platinum collection of small molecules (˜3,2×10⁵ commercially available compounds). Positive hits were filtered by different chemoinformatics tools to predict, pan-assay interference (i.e. chemical compounds that could give false positive results in different high-throughput screens), pharmacodynamics, physicochemical and ADME (adsorption, distribution, metabolism and excretion) properties. Compounds were also clustered based on their chemical scaffold. These analyses generated a ranking list of candidate hits, from which 31 molecules were further selected for biological validation.

### In Vitro Selection of Positive Hits

PrP biogenesis follows a trafficking pathway typical of glycosylphosphatidylinositol (GPI)-anchored polypeptides. The protein is synthesized directly in the lumen of the endoplasmic reticulum (ER), where it folds and receives post-translational processing of the primary structure (removal of signal peptide and anchoring of the GPI moiety) as well as the addition of two N-linked glycosylation chains (at Asn-181 and Asn-197). In principle, a compound binding to a PrP folding intermediate may produce a long-living, immature conformer that could be recognized by the ER quality control (ERQC) machinery, leading to its degradation by the ER-associated clearance pathway and/or by lysosome-dependent autophagy. Following this principle, the 31 putative ligands were directly tested in cells for their ability to lower the expression of PrP at a post-translational level. Each compound was administered for 24 h at different concentrations (1-3-10-30 μM) to HEK293 cells stably expressing mouse PrP. The expression and/or post-translational alterations of PrP were detected by Western blotting. Compounds showing an ability to lower the amount or alter the post-translational processing of PrP (Z 30%) at a least one concentration were tested against a control protein, NEGR-1. This GPI-anchored molecule follows the same expression pathway of PrP, thus representing an ideal control to evaluate compound specificity.
In addition, the cytotoxicity of each molecule was also evaluated in the same range of concentrations, by employing the MTT assay.
The screening activity performed led to the selection of the four compounds above referred as SM875, SM930, SM940 and SM950.

### EXAMPLE 2

### Chemical synthesis of SM875

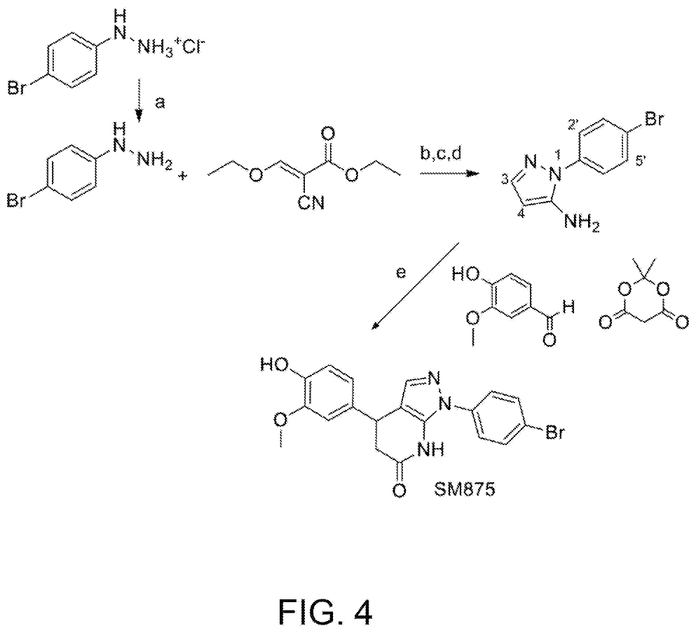

The target product SM875 was obtained according the synthetic strategy reported in FIG. 4. The sequence involves the preparation of the precursor 1-(4-bromophenyl)-1H-pyrazol-5-amine [1], which was used in a following three-component reaction with 4-hydroxy-3-methoxybenzaldehyde and Meldrum acid (2,2-dimethyl-1,3-dioxane-4,6-dione) according to a modified method by Zeng et al. [2]. The 1-(4-bromophenyl)-1H-pyrazol-5-amine was synthesized starting from 4-(bromophenyl) hydrazine that was obtained from the commercial hydrochloride by treatment with a saturated NaHCO₃aqueous solution (50 mL), followed by dichloromethane extraction (50 mL×3), treatment with anhydrous Na₂SO₄ and evaporation. To a magnetically stirred solution of 4-(bromophenyl)hydrazine (100 mg, 0.53 mmol, in 5 mL ethanol 5), (E)-ethyl 2-cyano-3-ethoxyacrylate (89.6 mg, 0.53 mmol) was added and refluxed for 2 h. The reaction mixture was concentrated in vacuo, the residue was suspended in 1:1 methanol/2M NaOH aq. solution (5 mL) and refluxed for 1 h. After cooling, the mixture was neutralized with 1M HCl aq. solution (5 mL) and concentrated in vacuo using a water bath at 40° C. The residue was heated at 180° C. for 10 min, suspended in ethanol after cooling and stored overnight at 4° C. The supernatant was recovered and concentrated to give a residue which was stirred in the presence of a NaHCO₃solution (10 mL). Extraction with ethyl acetate (10 mL×3), followed by the treatment with anhydrous Na₂SO₄ of the combined organic phases and concentration in vacuo gave the product (79 mg, 61%), which was used in the following three component reaction. The successful synthesis of 1-(4-bromophenyl)-1H-pyrazol-5-amine was verified by 1H-NMR [δH 7.58(d, J 8.7 Hz, H-3′ and H-5′), 7.47(d, J 8.7 Hz, H-2′ and H-6′), 7.41(s, H-3), 5.62 (s, H-4)] and ESIMS (m/z 239.8 [M+H]+). In the last reaction step, 1-(4-bromo-phenyl)-1H-pyrazol-5-amine (79 mg, 0.33 mmol), 4-hydroxy-3-methoxybenzaldehyde (41 mg, 0.27 mmol) and 2,2-dimethyl-1,3-dioxane-4,6-dione (46 mg, 0.32 mmol) in ethanol (5 mL) were refluxed under stirring for 2.5 h, or alternatively by replacing the conventional heating with microwave irradiation at 110° C. for 1 h. The reaction mixture was then cooled to room temperature and dried in vacuum. The raw product was purified by silica preparative thin layer chromatography (PLC) eluting with n-hexane/ethyl acetate (1:1). The band collected at retention factor 0.4 was first used for structural characterization and then injected into preparative HPLC (RP-18 column, acetonitrile/water 1:1, UV detection at 254 nm, flow 5 mL·min⁻¹, retention time 4.5 min) to give the target product (for use on cell cultures) as a white powder after evaporation of the eluent: 34 mg, 25% with reflux in ethanol; a yield of ˜25% is also obtained with microwave irradiation.

### Structural Characterization of SM875.

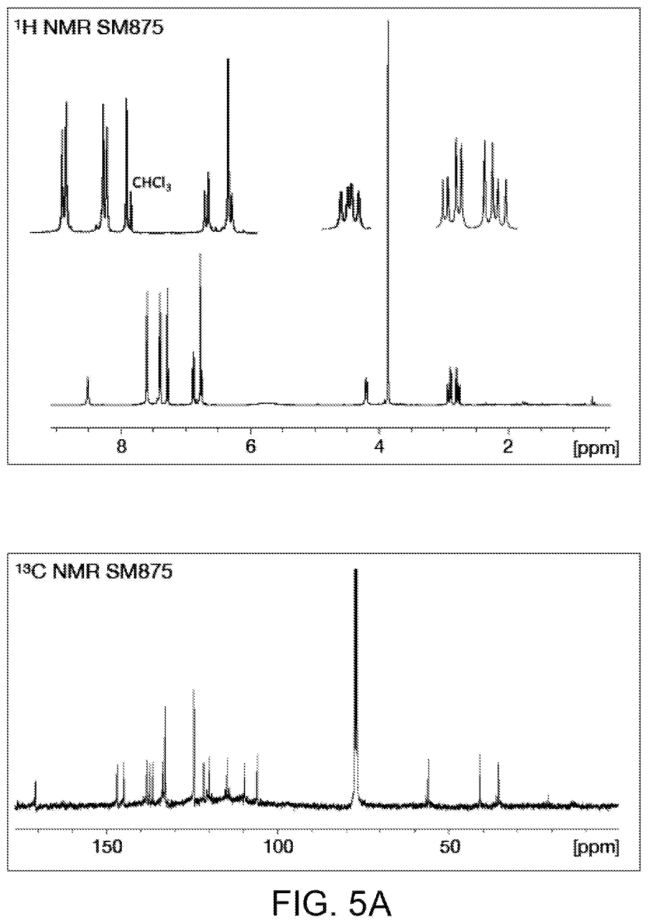

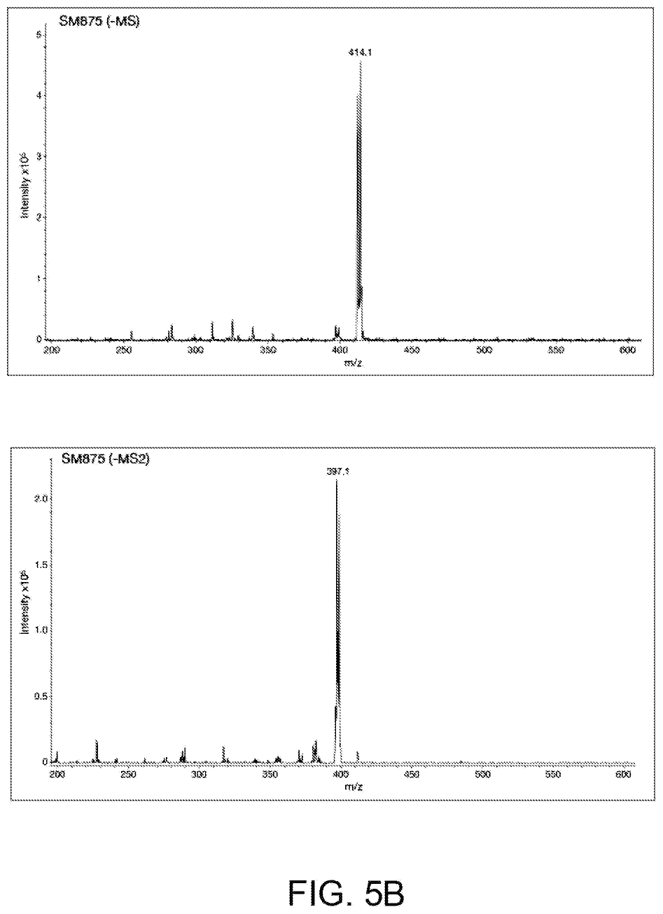

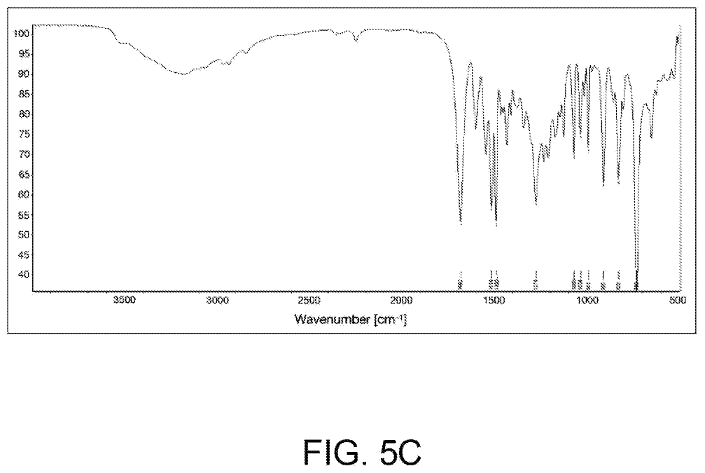

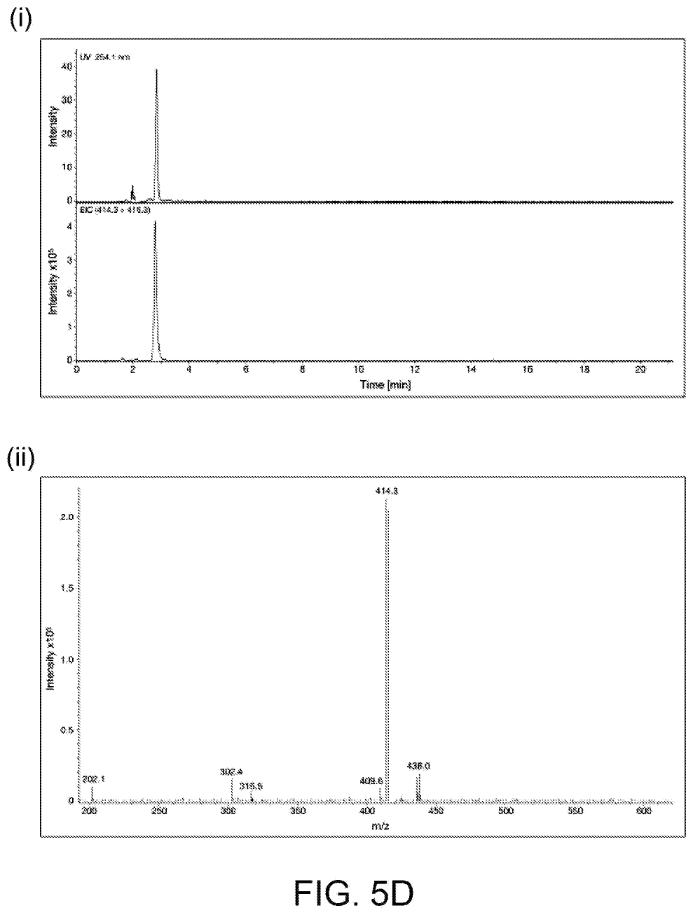

NMR, ESI-MS spectra, together with LC-MS (UV, EIC and MS) are reported in FIG. 5, A-D. Reported high resolution mass spectrometry values correspond to the average of 100 measures: HRESI(+)MS: m/z 414.04426±0.00126 [M+H]+(calculated for C₁₉H₁₇⁷⁹BrN₃O₃, 414.04478); HRESI(+)MS/MS (414): m/z 399.02055±0.00190 [M+H]+(calculated for C₁₈H₁₄⁷⁹BrN₃O₃, 399.02131); m/z 372.03349±0.00195 (calculated for C₁₇H₁₅⁷⁹BrN₃O₂, 372.03422); m/z 289.99184±0.00175 (calculated for C₁₂H₉⁷⁹BrN₃O, 372.03422).

### Characterization of Therapeutic Activity of SM875

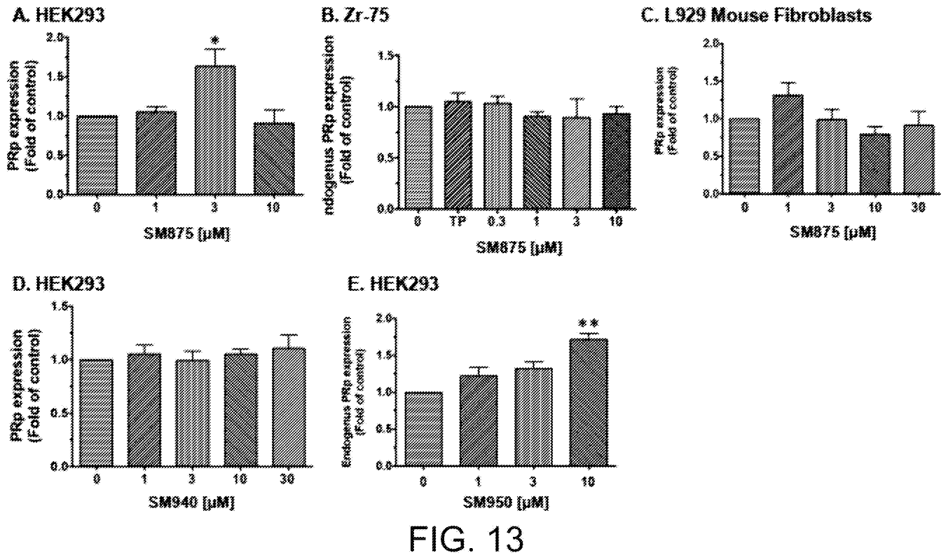

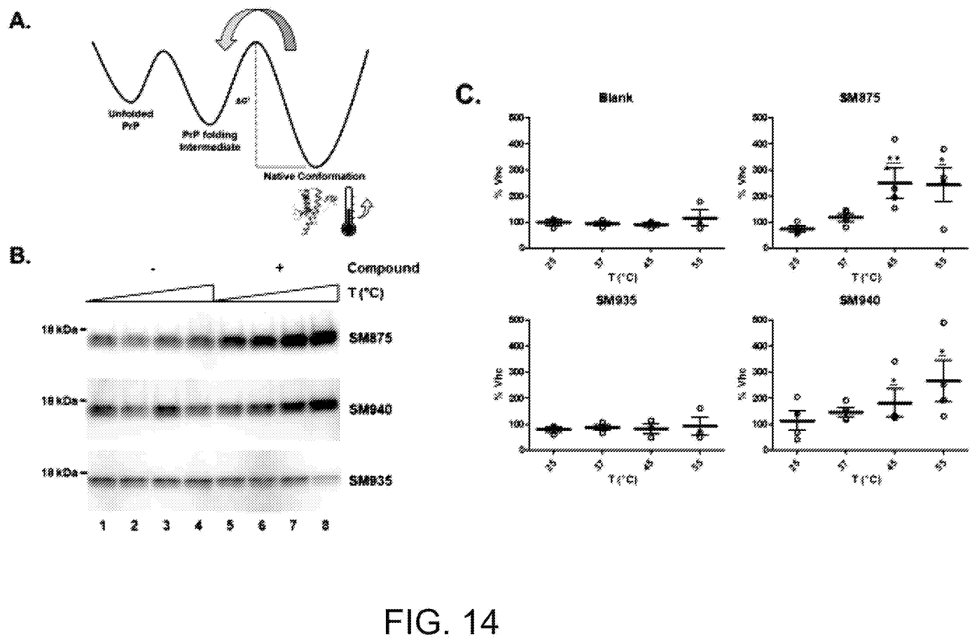

Compound SM875 (shown in FIG. 6A) induces a dose-dependent decrease of PrP^{C} expression, accompanied by a progressive shift of the signal from the expected ˜35 kDa of full-length, di-glycosylated native PrP^{C}, to lower (>28 kDa) molecular weight bands, likely corresponding to cleaved PrP forms. These effects were already evident at concentrations as low as 3 μM (FIG. 6B, lanes 7-10). Importantly, SM875 did not produce any effect on NEGR-1, a non-relevant GPI-anchored protein synthesized in the ER like PrP^{C} (FIG. 6B). HEK293 cells (ATCC, CRL-1573) were stably transfected with mouse PrP^{C} or NEGR-1 were exposed to different concentrations of SM875 (shown) or vehicle (DMSO, volume equivalent) for 24 h, lysed and analyzed by Western blotting. Signals were detected by using a specific anti-PrP^{C} or anti-NEGR-1 primary antibody, and relevant HRP-coupled secondary antibodies, and revealed using a ChemiDoc Touch Imaging System (Bio-Rad, CA, USA) Similar effects were obtained in ZR-75 cells, a human breast cancer cell line endogenously expressing PrP^{C} (FIG. 6D). Importantly, the decrease of PrP^{C} expression was not accompanied by a decrease of its mRNA, as assayed by RT-PCR (FIG. 13), demonstrating that the compound acts at a post-translational level.

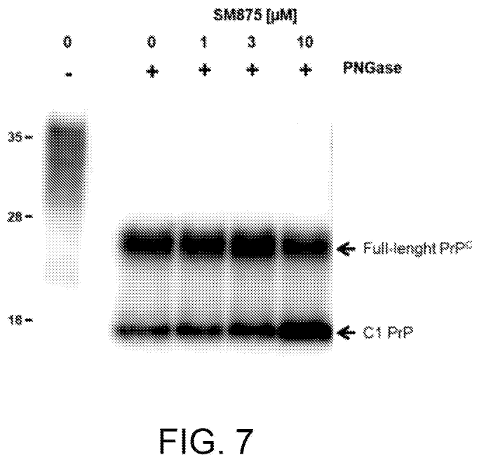

Next, it was checked whether SM875 could alter the post-translational processing of PrP^{C}. The protein is known to be physiologically subjected to a primary post-translational cleavage named alpha-cleavage, which divides the N- and C-terminal domains of PrP^{C}, producing a membrane-bound C-terminal half (residues ˜111-231) and a soluble fragment called N1 (residues ˜23-110). The exact cleavage location, as well as the precise identity of the responsible enzymes are still debated. In order to test the effect of SM875 on the alpha-cleavage, HEK293 cells stably expressing PrP^{C} were incubated with different concentrations of compound (1-10 μM) for 24 h. Cell lysates were then treated with the enzyme PNGase-F to remove sugar moieties, and analyzed by Western blotting (FIG. 7). Upon treatment with SM875, a dose-dependent increase of Cl PrP was observed, accompanied by a proportional decrease of full-length PrP^{C}, indicating that the compound increases alpha-cleavage. Collectively, these data indicate that SM875 induces a specific, dose-dependent decrease of full-length, native PrP^{C} by promoting its degradation.

### SM875 Decreases the Amount of Full-Length PrP^{C} at the Cell Surface

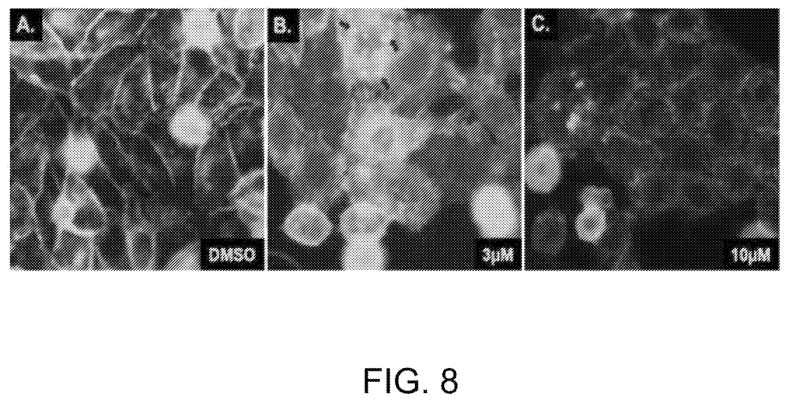

Based on the observed effects of SM875 on PrP^{C} expression, it was hypothesized that the compound could also decrease the total amount of the protein at the cell surface. In order to directly test this hypothesis, HEK293 cells stably transfected with a PrP^{C} form tagged with a monomerized EGFP molecule at its N-terminus (EGFP-PrP^{C}) were seeded on an 8-well chamber slide and incubated with SM875 (FIG. 8, B and C) or vehicle (DMSO) control (FIG. 8 A). Coverslips were mounted with a gel mount (Sigma Aldrich), and visualized with a Cell-R imaging station (Olympus) coupled to an inverted microscope (IX 81, Olympus). Fluorescent signals deriving from EGFP were acquired with a high-resolution camera (ORCA) equipped with a 488 nm excitation filter, and an emission filter with a range of 510±40 nm. In control conditions, EGFP-PrP^{C} localizes almost entirely at the plasma membrane, giving rise to a typical “honeycomb-like” staining of the cell surface. Compounds altering PrP^{C} trafficking, as the phenothiazine derivative chlorpromazine, have previously been shown to alter such localization pattern [12]. Importantly, incubation with SM875 for 24 h induced a drastic reduction of cell surface EGFP-PrP^{C}, at the same concentrations (3-10 μM) at which the molecule produced effects on PrP^{C} expression and processing (FIG. 8). Interestingly, in few cells treated with SM875 it was observed the appearance of dot-like structures, possibly indicating the accumulation of EGFP-PrP^{C} in specific intracellular compartments, compatible with a model in which the protein is removed from the ER and sent to intracellular degradation.

### Generation and Characterization of SM898, a Derivative of SM875

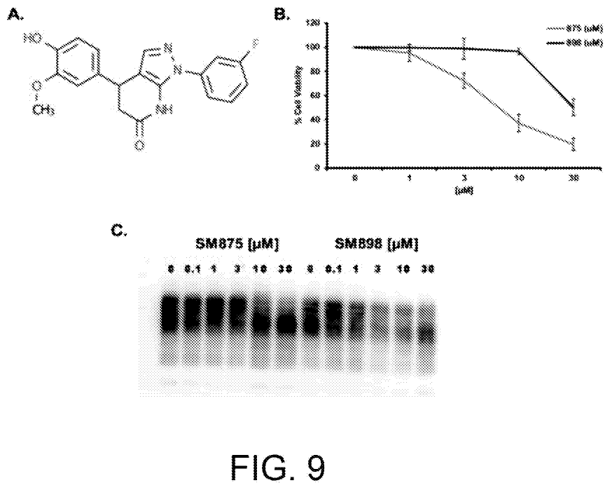

In several experiments it was noticed an evident cytotoxicity of SM875 at concentrations near those at which the compound exerted the maximum biological effect. In an attempt to reduce the cytotoxicity of SM875, a fluorinated derivative was designed (called SM898, FIG. 9A). SM898 showed a strongly reduced (˜3 times) cytotoxicity, as assayed in HEK293 cells exposed to each compound for 48 h, and subsequently analyzed by MTT assay (FIG. 9B; estimated median lethal dose, LD50, was 4.6 μM for SM875, and 37.9 μM for SM898). Importantly, no appreciable differences were observed between SM875 and SM898 in their ability to reduce PrP^{C} expression (FIG. 9C). Collectively, these results indicate that SM898 is less toxic than its parent compound, while retaining its biological activity.

### EXAMPLE 3

### Characterization of Therapeutic Activity of SM930, SM940 and SM950

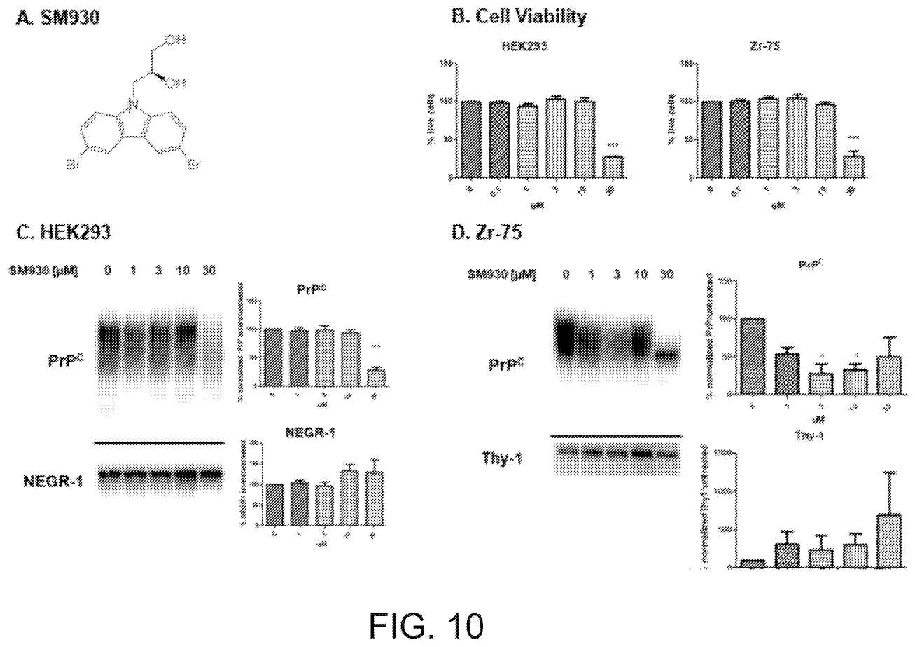

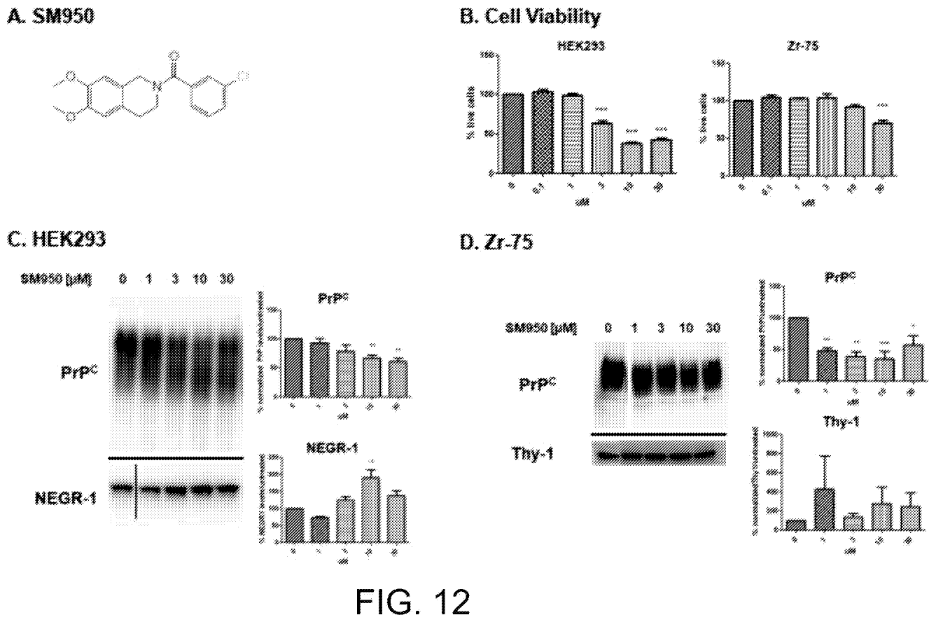

Three additional compounds, named SM930, SM940 and SM950 (shown in FIGS. 10, 11, and 12 respectively) induced a specific reduction of PrP^{C} levels in cells, as assayed by Western Blotting analysis, following the same experimental protocol described above for SM875. These molecules were also analyzed for intrinsic toxicity by MTT essay. SM930 suppressed PrP^{C} expression, but not NEGR-1, at the highest concentration tested (30 μM) when tested in stably-transfected HEK293 cells. The molecule also induced cell death at the highest concentration tested (30 μM). SM940 suppressed PrP^{C} expression, but not NEGR-1, when tested in stably-transfected HEK293 cells, starting from a concentration of 3 μM. Cell viability assay in HEK293 and Zr-75 cells, as assayed by MTT, showed that SM940 did not induce significant cell death even at the highest concentration tested (30 μM). Finally, SM950 suppressed PrP^{C} expression, but not NEGR-1, when tested in stably-transfected HEK293 cells, starting from a concentration of 10 μM. Cell viability assay in HEK293 and Zr-75 cells, as assayed by MTT, showed that this compound induced significant cell death already at a concentration of 3 μM in HEK293 cells, although the molecule was much less toxic when tested in Zr-75 cells (where it showed modest toxicity only at 30 μM). Conversely, none of the compounds decreased the expression of control protein NEGR-1 at any concentration.

### EXAMPLE 4

### Decreasing Activity of SM875, SM940 and SM950 in the Levels of PrP^{C} mRNA

In order to rule out the possibility that SM875, SM940 and SM950 acted by decreasing the levels of PrP^{C} mRNA, the inventors carried out real time PCR analysis of the PrP^{C} transcript in different cell lines upon compound treatment (FIG. 13). The results showed that none of these molecules suppress PrP^{C} expression by decreasing the levels of its mRNA.

### EXAMPLE 5

### Compounds SM875 and SM940 Induce the Aggregation of Recombinant PrP^{C} in a Temperature-Dependent Fashion

Compounds SM940 and SM875 have been identified based on the PPI-FIT rationale of targeting a folding intermediate of PrP^{C}. In order to observe a direct effect of these molecules on PrP^{C} in vitro, an experimental paradigm was designed in which natively folded PrP^{C} is heated at different temperatures with the hope to provide enough energy to allow its conformational transition to the predicted folding intermediate (FIG. 14). If the folding intermediate appears as a consequence of the raising temperature, then the inventors predicted that compounds of the invention, for instance SM875 or SM940, should be able to bind it, causing its stabilization and thus likely inducing its aggregation, since FI-PrP exposes to the solvent hydrophobic residues which instead are buried inside the natively folded protein core of PrP^{C}.
Recombinant PrP aggregation was evaluated by a detergent-insolubility assay. Compounds SM875 and SM940 increased the amount of detergent-insoluble recombinant PrP in a temperature-dependent fashion; conversely, no effects were detected in untreated controls, or when the protein was incubated with molecule SM935 (which was found negative after the cell-based screen and used here as a negative control), as shown by western blot. These results indicate that compounds SM875 and SM940 bind to a folding intermediate of PrP^{C} (i.e. FI-PrP). Statistical differences were estimated by one-way Anova, Dunnet post-hoc test. p values are: *<0.05, **<0.01.

### EXAMPLE 6

### SM875 Induces the Degradation of PrP^{C} in the Lysosomal Compartment

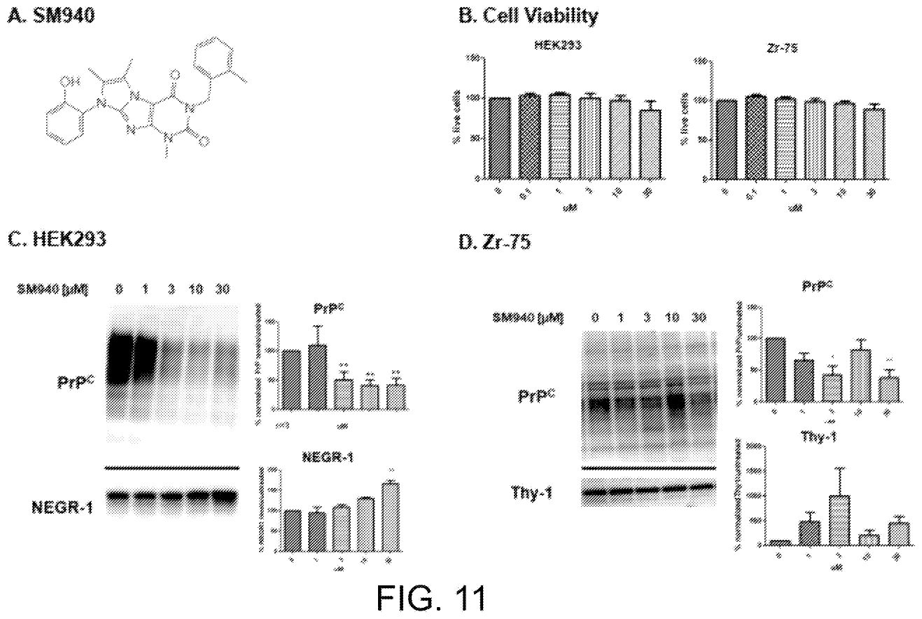

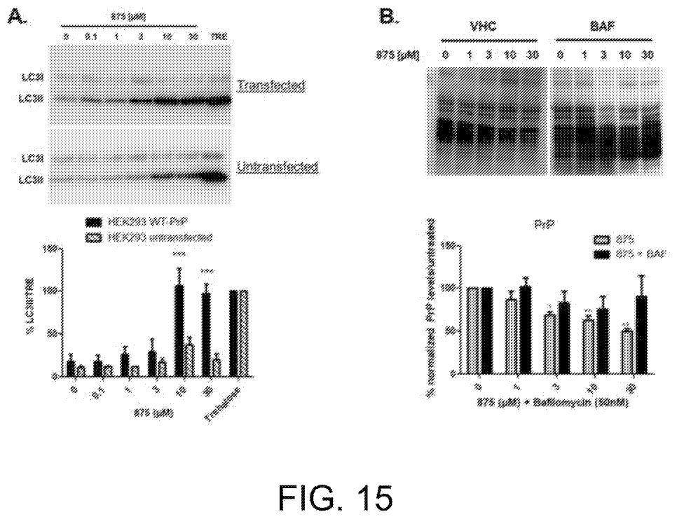

The inventors sought to test the possibility that the molecule SM875 and its derivatives could induce the degradation of PrP^{C} via lysosome-dependent autophagy. First, they observed that SM875 induces the activation of autophagy in cells in a PrP^{C}-dependent fashion, as assayed by detecting autophagy marker LC3-II (FIG. 15 A). Next, they found that autophagy inhibitor Bafilomycin Al rescues SM875-induced PrP^{C} decrease in cells (FIG. 11 B). Collectively, these results suggest that SM875 promotes the degradation of PrP^{C} by the lysosome-dependent autophagy pathway.

### EXAMPLE 7

### SM875 Inhibits Prion Replication in Mouse Fibroblasts

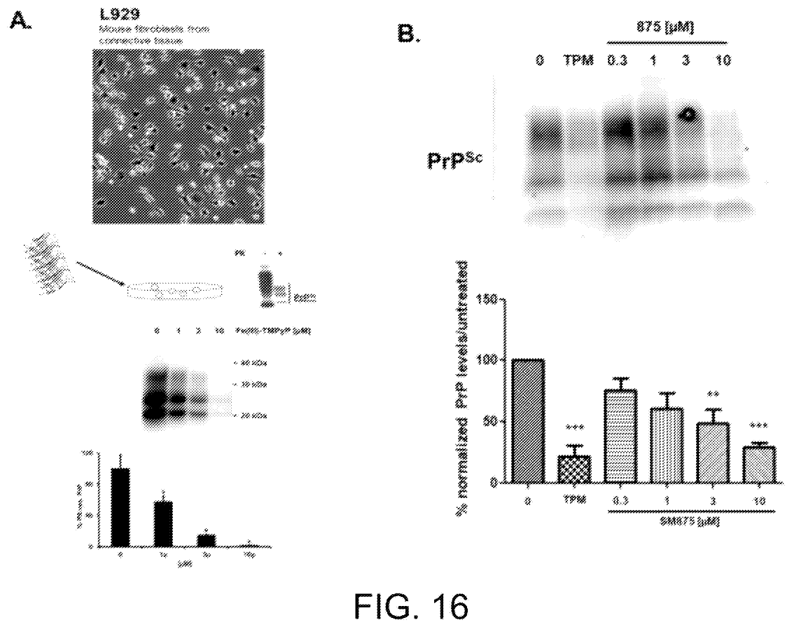

Following the observation that SM875 suppresses the expression of PrP^{C}, the substrate for prion replication, the inventors sought to test the ability of this molecule to lower the amount of proteinase-K (PK)-resistant PrP molecules in mouse fibroblasts persistently infected with the Rocky Mountain Laboratories (RML) prion strain. As shown in FIG. 16, they found that compound SM875 inhibits prion replication in a dose-dependent fashion, decreasing prion loads similarly to compound TPM, a previously reported potent anti-prion compound (Massignan et al. SciRep 2016).

### EXAMPLE 8

### Chemical Synthesis of SM875 Analogues

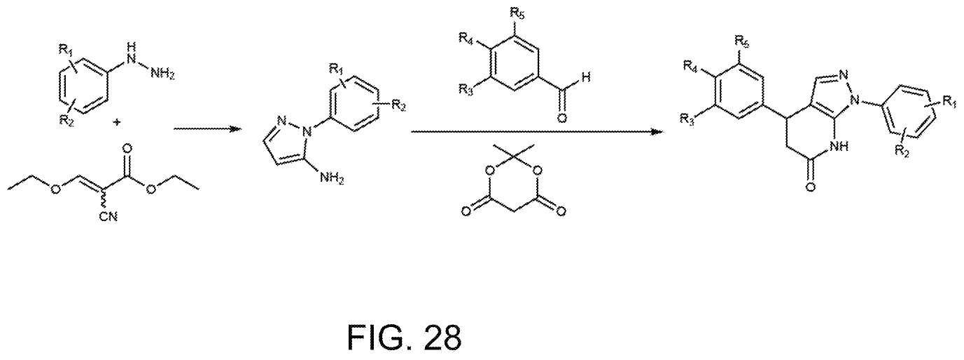

The scheme for the synthesis of compound SM875 (see above paragraph “Chemical Synthesis of SM875”) was used to obtain 24 analogs, using the following commercially available substituted phenyl-hydrazines (groups R1 and R2) and substituted aryl-aldehydes (groups R3, R4, R5) and more particularly the following reagents (Sigma Aldrich & Alfa Aesar): phenylhydrazine hydrochloride, 4-(fluorophenyl)-hydrazine hydrochloride, 4-(chlorophenyl)-hydrazine hydrochloride, 4-(iodophenyl)hydrazine, p-tolylhydrazine hydrochloride, 4-(trifluoromethyl)-phenylhydrazine, 3-(bromophenyl)hydrazine hydrochloride, 2-(bromophenyl)-hydrazine hydrochloride, 2-(fluorophenyl)-hydrazine hydrochloride, 2,4-(difluorophenyl)-hydrazine hydrochloride, 3-chloro-4-fluorophenylhydrazine, benzaldehyde,4-hydroxybenzaldehyde, 4-phenoxybenzaldehyde,3-hydroxy-4-methoxybenzaldehyde, 4-ethoxy-3-methoxy-benzaldehyde, 1,4-benzodioxan-6-carboxaldehyde, 3-mnethoxy-4-Benziloxybenzaldehyde, and 2,3,4-trimnethoxybenzaldehyde. See the scheme of FIG. 28, wherein:

| Compound | R1 | R2 | R3 | R4 | R5 |
|---|---|---|---|---|---|
| EP3 | H | H | OCH₃ | OH | H |
| GS1 | p-F | H | OCH₃ | OH | H |
| EP1 | p-Cl | H | OCH₃ | OH | H |
| GC8 | p-I | H | OCH₃ | OH | H |
| EP2 | p-CH₃ | H | OCH₃ | OH | H |
| GS2 | p-CF₃ | H | OCH₃ | OH | H |
| GC5 | m-Br | H | OCH₃ | OH | H |
| GC7 | o-F | H | OCH₃ | OH | H |
| GC9 | p-F | o-F | OCH₃ | OH | H |
| LC5 | p-Br | H | H | H | H |
| LC6 | p-Br | H | H | OH | H |
| LC2 | p-Br | H | H | OPh | H |
| LC1 | p-Br | H | OH | OCH₃ | H |
| CP3 | p-Br | H | OC₂H₅ | OCH₃ | H |
| CP2 | p-Br | H | 3,4-OCH₂CH₂O— | 3,4-OCH₂CH₂O— | H |
| LC3 | p-Br | H | OCH₃ | OCH₂Ph | H |
| CP1 | p-Br | H | OCH₃ | OCH₃ | OCH₃ |
| GC1 | m-Br | H | OH | OCH₃ | H |
| GC8 | o-F | H | OH | OCH₃ | H |
| GC4 | p-F | o-F | OH | OCH₃ | H |
| GC3 | p-F | m-Cl | OH | OCH₃ | H |
| CG10 | p-I | H | OH | OCH₃ | H |
| GIO1 | p-I | H | 3,4-OCH₂CH₂O— | 3,4-OCH₂CH₂O— | H |

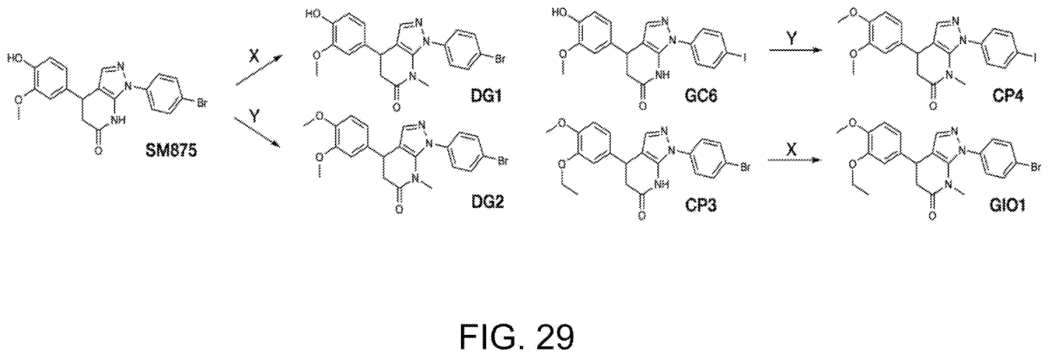

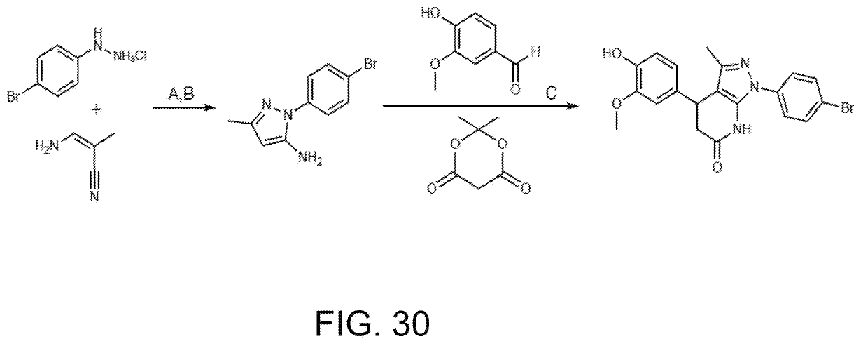

Four further derivatives were obtained by methylation of SM875 and analogues CG6 and CP3. In this process, ˜5 mg of starting compound was added to a solution of acetone/iodomethane 1:1. Subsequently, 50 mg of K₂CO₃ was added to the mixture and let react for 24 h, to obtain the single methylation on the lactam nitrogen, or for 48 h to obtain additional methylation on hydroxyl groups (where present). The soluble product was then obtained by filtering out the precipitated salt and concentrated in vacuo.
As depicted in FIG. 29, the four methyl-derivatives were synthesized: two starting from SM875, one starting from CP3 and one starting from GC6. Reaction conditions: (X)=CH₃I, K₂CO₃, 24 h. (Y)=CH₃I, K₂CO₃, 48 h. Method from Selvaraju et al.237
A further analogue has been obtained by synthesizing the modified precursor 1-(4-bromophenyl)-3-methyl-1H-pyrazol-5-amine that was used instead of the 1-(4-bromophenyl)-1H-pyrazol-5-amine to perform the three-component reaction. In particular, analogue DG3 was obtained by employing the modified precursor 1-(4-bromophenyl)-3-methyl-1H-pyrazol-5-amine instead of the 1-(4-bromophenyl)-1H-pyrazol-5-amine. This alternative precursor was obtained by reacting 4-(bromophenyl)-hydrazine hydrochloride and 3-aminoacrylonitrile in 0.6 mL 2M HCl aqueous solution at 100° C. by microwave irradiation for 1 h. The reaction mixture was neutralized with aqueous NaOH solution (0.25 M, ˜6 mL) and after 15 min stirring it was extracted in dichloromethane. The combined organic phases were treated with anhydrous Na₂SO₄ and concentrated in vacuo. The successful synthesis of 1-(4-bromo-phenyl)-3-methyl-1H-pyrazol-5-amine was verified by ¹H-NMR analysis. This compound was then employed as a reagent for the three-component reactions, as previously described, to yield DG3.
In particular, the following reagents and conditions have been employed for the synthesis of DG3: (A) HCl 2M aqueous solution, 100° C. by microwave irradiation for 1 h. (B) Neutralization by NaOH solution and extraction in dichloromethane. (C) 2.5 h reflux or alternatively 110° C. in a microwave reactor for 1 h in ethanol.
All the synthesized analogues were purified using preparative HPLC technique before cell-based testing.

### EXAMPLE 9

### Comparison of Efficacy for the Analogue Compounds

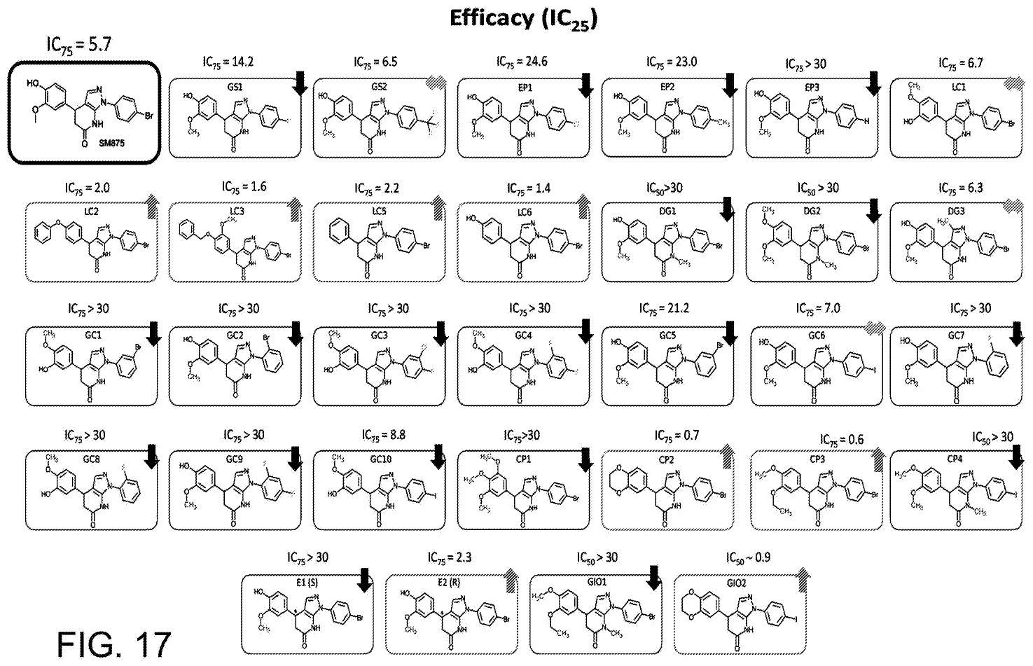

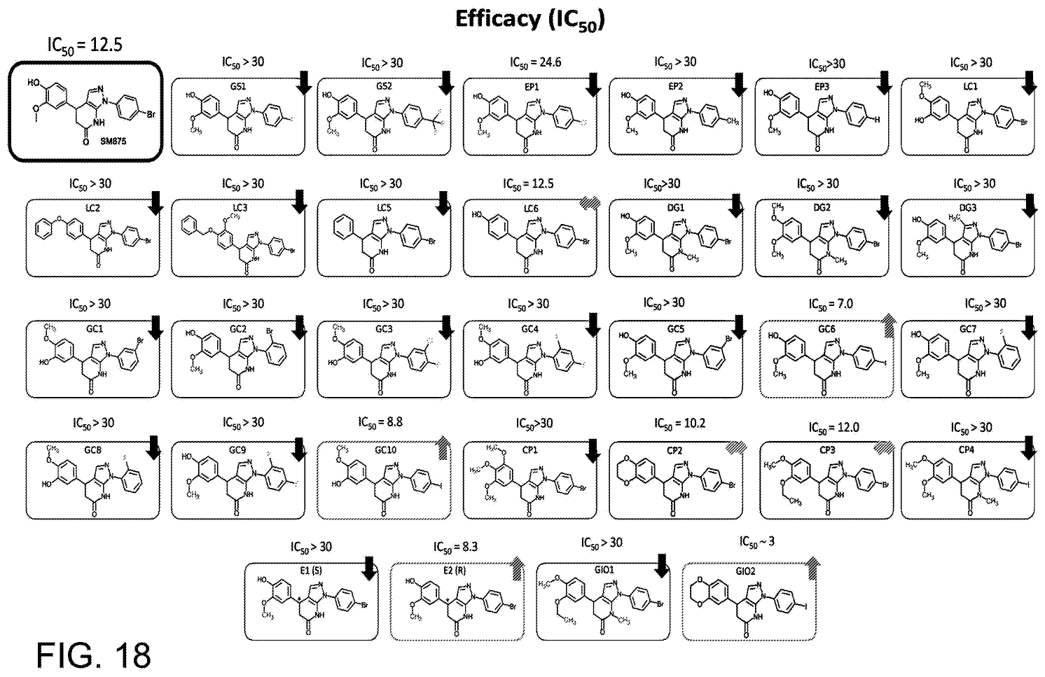

The effect on the expression of PrP of different chemical analogs was tested by western blotting in HEK293 cells stably expressing mouse PrP. We identified several molecules decreasing (down arrows) or not altering PrP-suppressing effects, as compared to the parent compound SM875. Conversely, few molecules suppressed PrP expression at lower concentrations (up arrows), as indicated by the IC₂₅ (FIG. 17) and IC₅₀ (FIG. 18) values reported above each compound.
The IC values are summarized in the following table.

| Compound | IC | Value μM | IC | Value μM |
|---|---|---|---|---|
| SM875 | IC50 | 12.5 | IC75 | 5.7 |
| GS1 | IC50 | >30 | IC75 | 14.2 |
| GS2 | IC50 | >30 | IC75 | 6.5 |
| EP1 | IC50 | 24.6 | IC75 | 24.6 |
| EP2 | IC50 | >30 | IC75 | 23.0 |
| EP3 | IC50 | >30 | IC75 | >30 |
| LC1 | IC50 | >30 | IC75 | 6.7 |
| LC2 | IC50 | >30 | IC75 | 2.0 |
| LC3 | IC50 | >30 | IC75 | 1.6 |
| LC5 | IC50 | >30 | IC75 | 2.2 |
| LC6 | IC50 | 12.5 | IC75 | 1.4 |
| DG1 | IC50 | >30 | IC50 | >30 |
| DG2 | IC50 | >30 | IC50 | >30 |
| DG3 | IC50 | >30 | IC75 | 6.3 |
| GC1 | IC50 | >30 | IC75 | >30 |
| GC2 | IC50 | >30 | IC75 | >30 |
| GC3 | IC50 | >30 | IC75 | >30 |
| GC4 | IC50 | >30 | IC75 | >30 |
| GC5 | IC50 | >30 | IC75 | 21.2 |
| GC6 | IC50 | 7.0 | IC75 | 7.0 |
| GC7 | IC50 | >30 | IC75 | >30 |
| GC8 | IC50 | >30 | IC75 | >30 |
| GC9 | IC50 | >30 | IC75 | >30 |
| GC10 | IC50 | 8.8 | IC75 | 8.8 |
| CP1 | IC50 | >30 | IC75 | >30 |
| CP2 | IC50 | 10.2 | IC75 | 0.7 |
| CP3 | IC50 | 12.0 | IC75 | 0.6 |
| CP4 | IC50 | >30 | IC50 | >30 |
| E1 (S) | IC50 | >30 | IC75 | >30 |
| E2 (R) | IC50 | 8.3 | IC75 | 2.3 |
| GIO1 | IC50 | >30 | IC50 | >30 |
| GIO2 | IC50 | ~3 | IC50 | ~0.9 |

### EXAMPLE 10

### Quantification of the Effect of Individual SM875

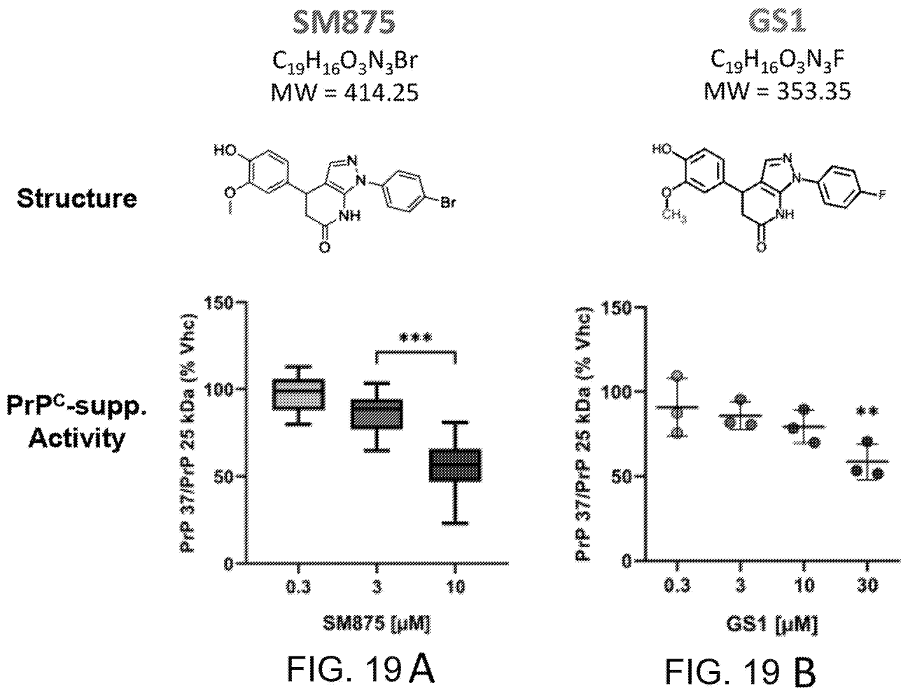

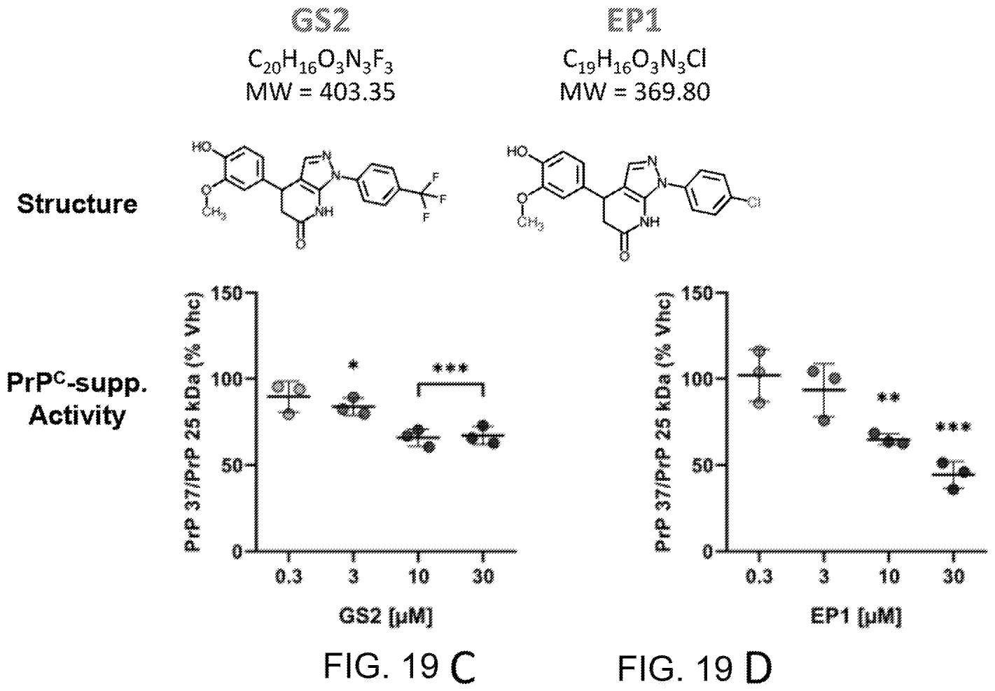

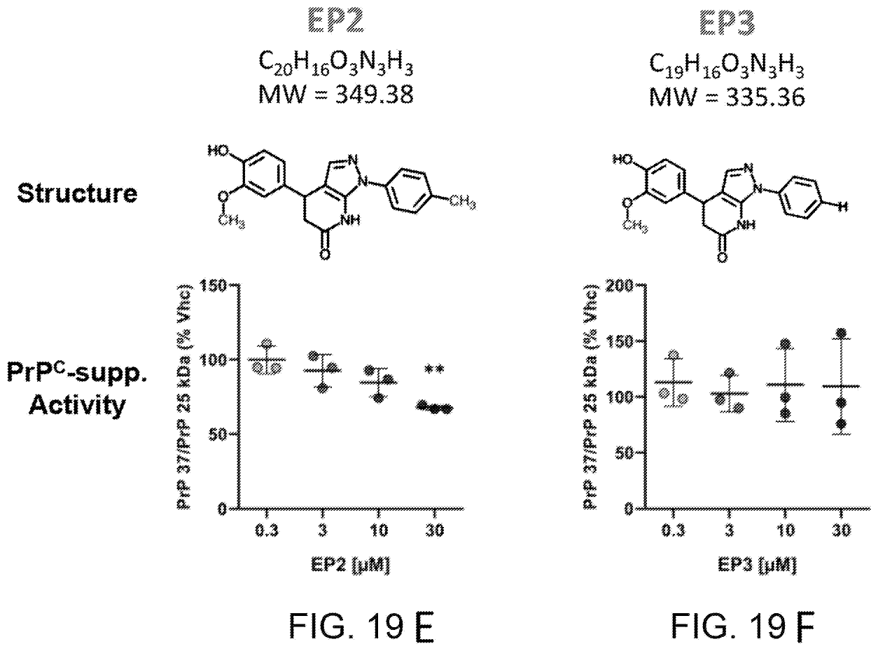

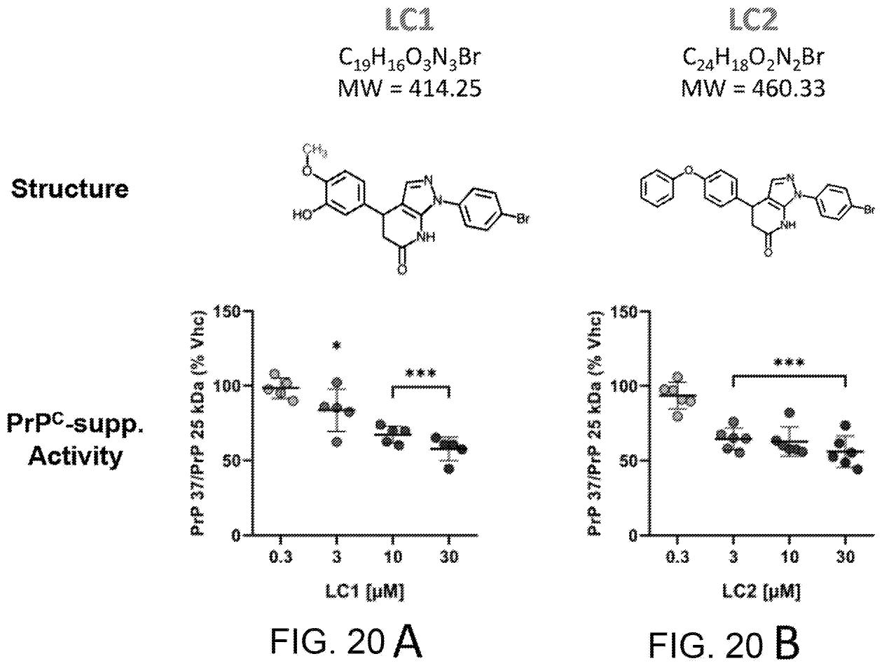

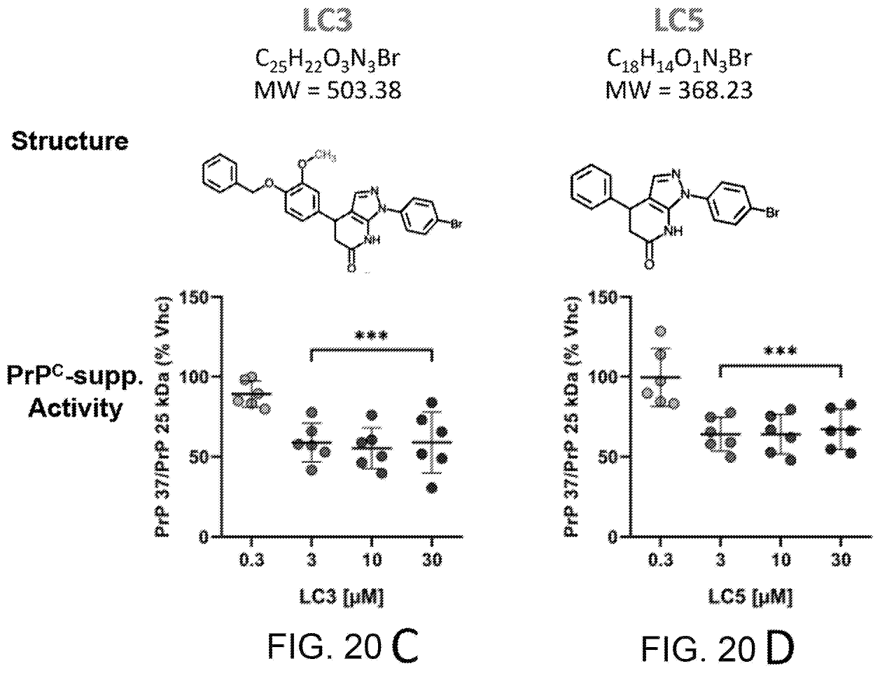

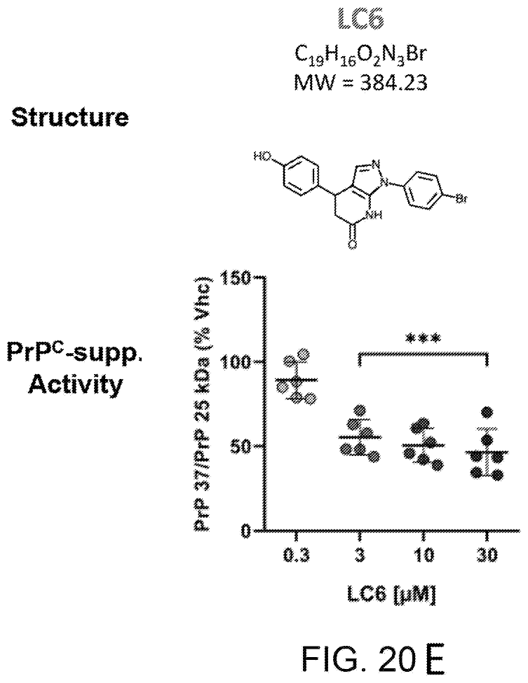

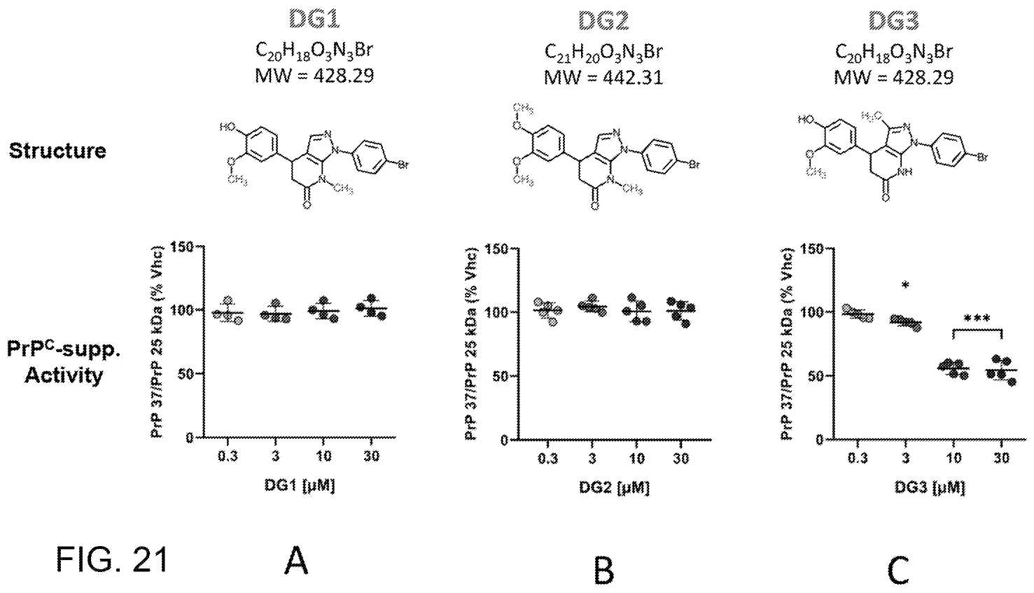

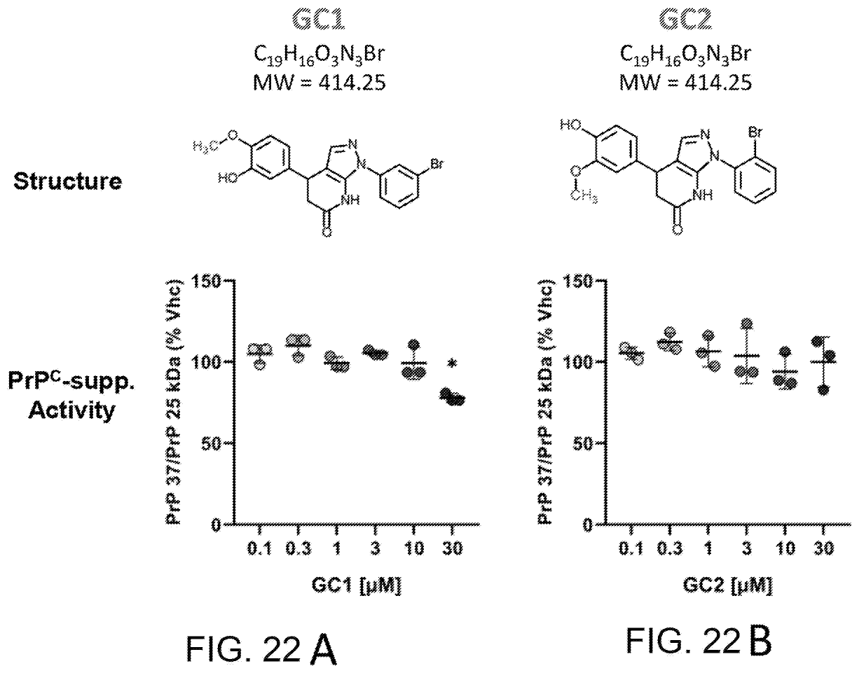

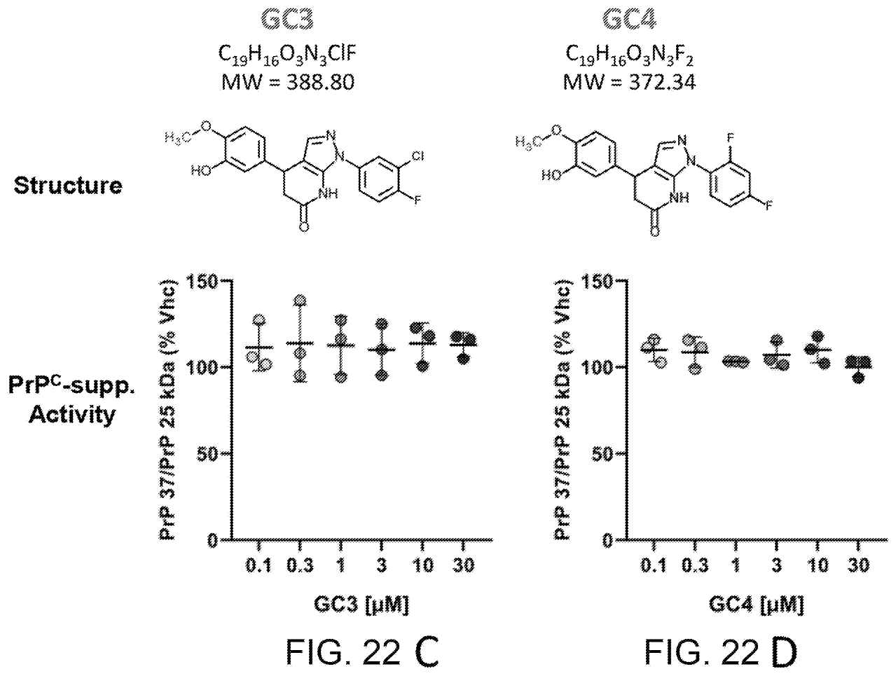

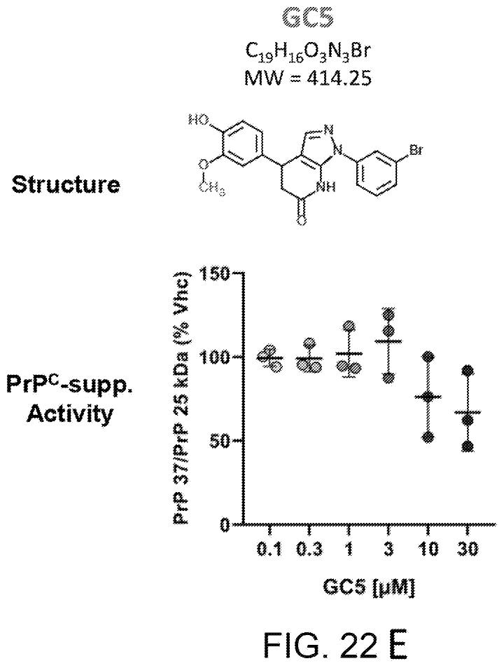

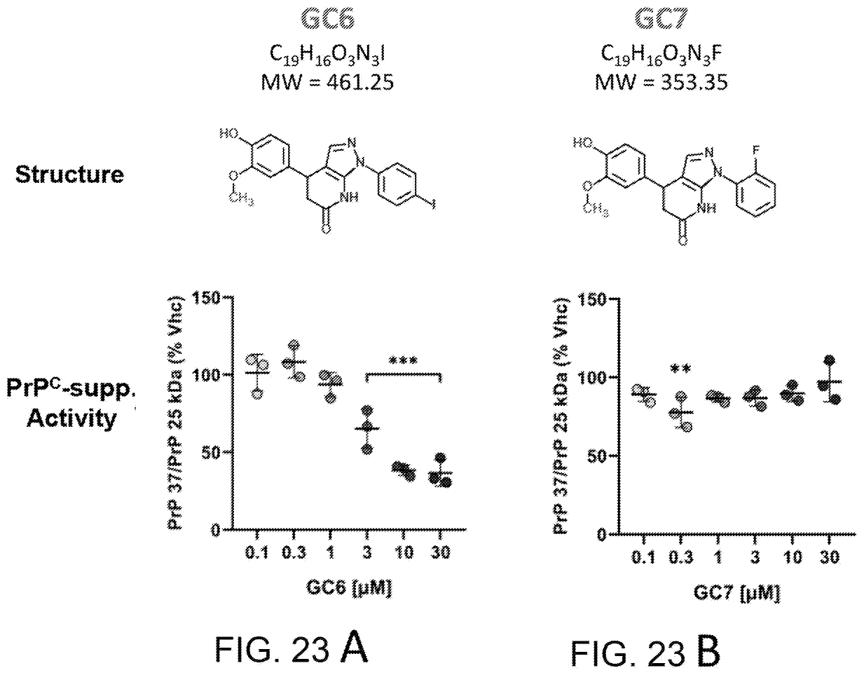

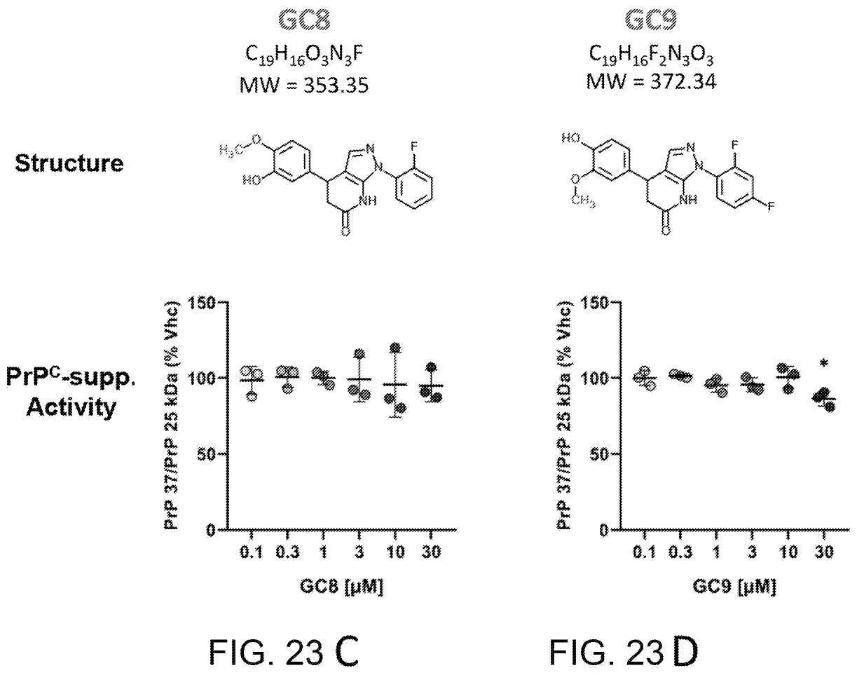

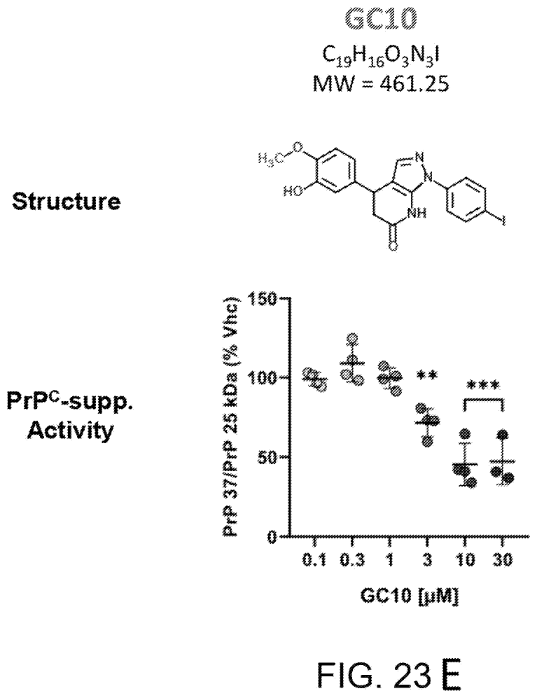

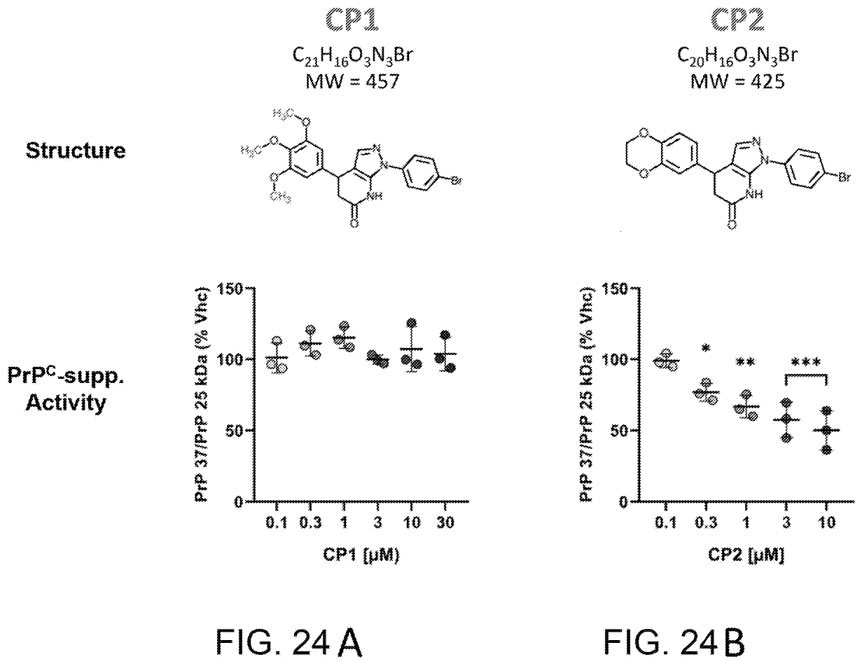

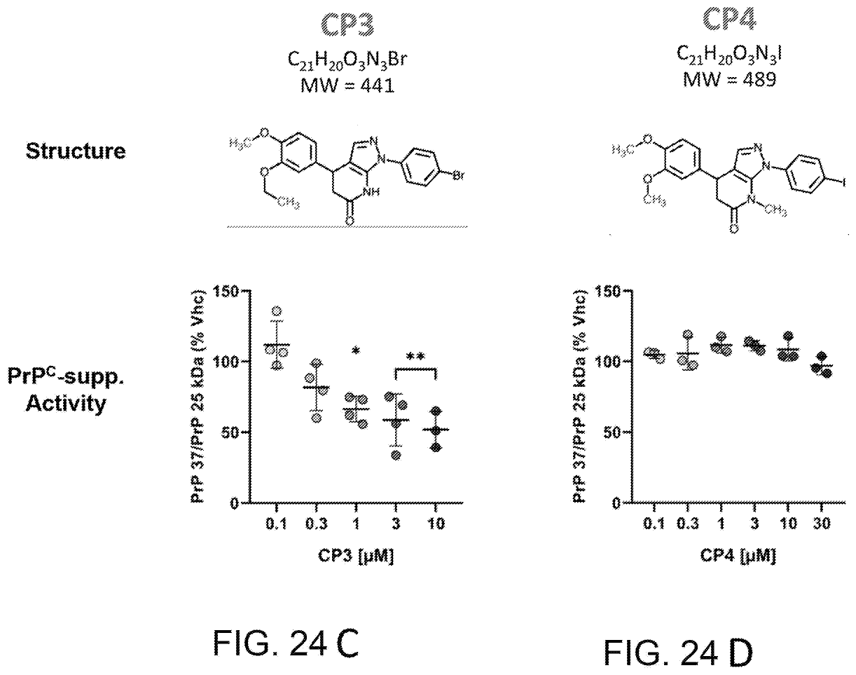

FIG. 19-25 include compound's reference name, molecular structure and a graph reporting the quantification of PrP levels. The data were collected in HEK293 cells (ATCC, CRL-1573) stably transfected with mouse PrP, exposed to different concentrations of each compound (indicated) or vehicle (DMSO, volume equivalent) for 48 h, lysed and analyzed by western blotting. Signals were detected by using specific anti-PrP primary antibody, a relevant HRP-coupled secondary antibody, and revealed using a ChemiDoc Touch Imaging System. Each graph shows the average densitometric quantification (±standard error, when applicable) of the levels of full-length PrP from different independent replicates (n≥3, with the exception of molecule GIO2, for which n=2). Each signal was normalized on the corresponding total protein lane (detected by UV, and allowed by the enhanced tryptophan fluorescence technology of stain-free gels) and expressed as the percentage of the level in vehicle (Vhc)-treated controls.

### EXAMPLE 11

### Evaluation of PrP-Suppressing Effects of Two SM875 Enantiomers

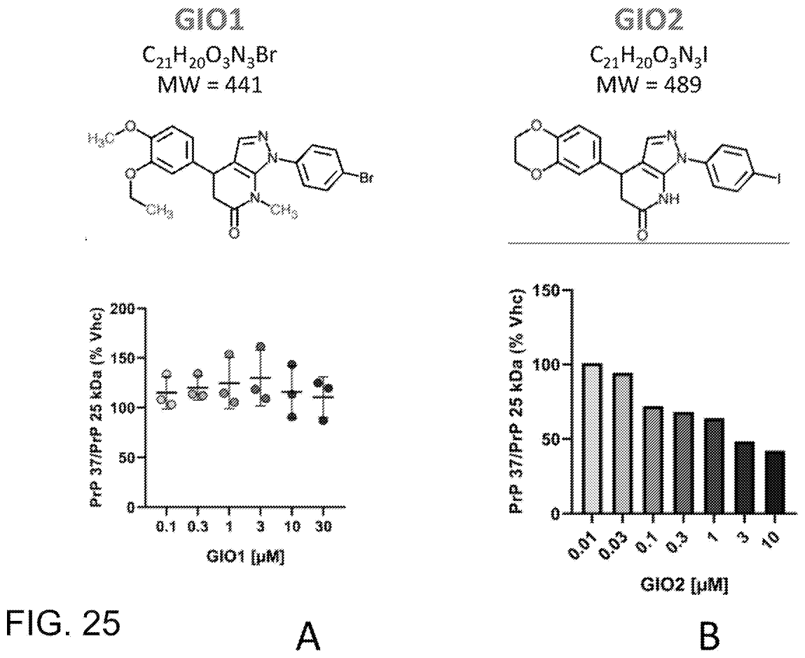

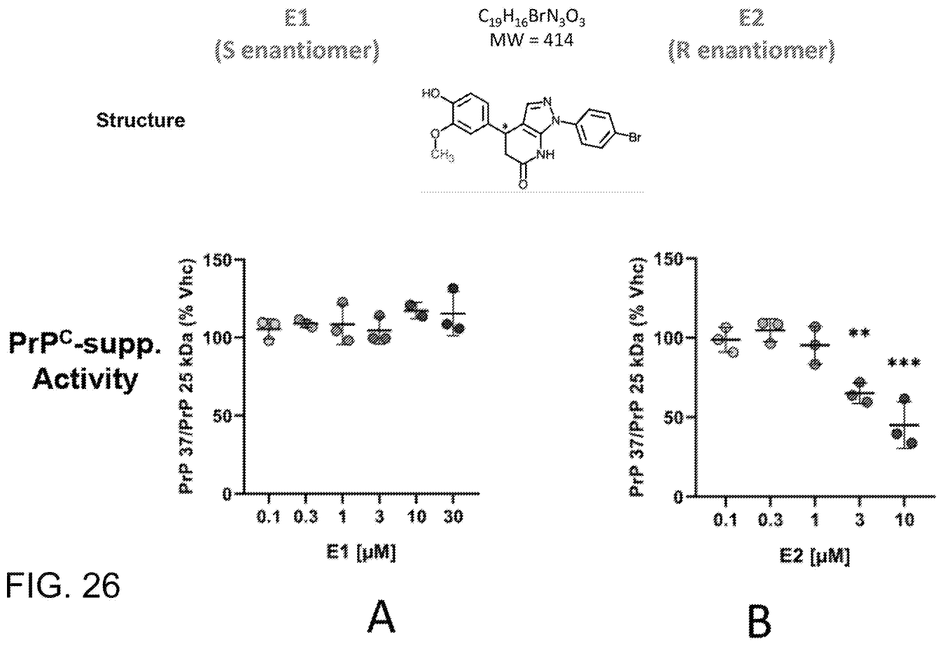

The PrP-lowering effects of each individual enantiomer of SM875 (the chiral center indicated by the asterisk) were tested as described in the previous Example. The analysis revealed that only one of the two enantiomers (E2, R configuration) retains the entire PrP-suppressing activity, suggesting that the binding of SM875 to the PrP folding intermediate is stereoselective. See FIG. 26.

### EXAMPLE 12

### Dose-Response Analysis of SM875 Derivatives

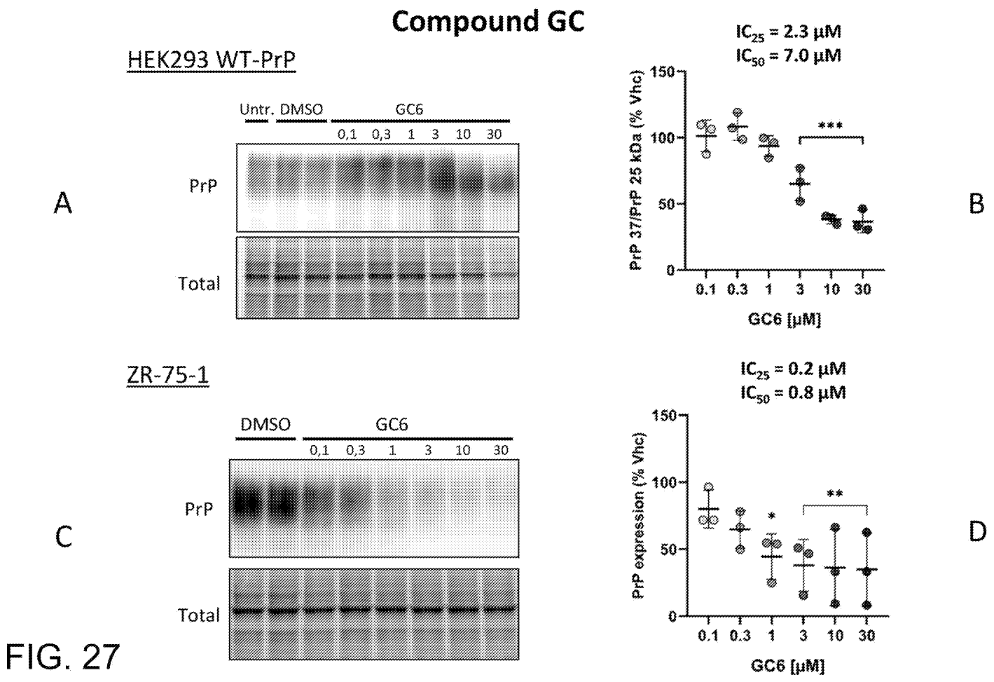

FIG. 27 shows a selected analogue (GC6) was tested at six different concentrations (0.1-30 μM) in HEK293 cells and ZR-75 human breast cancer cells, following the exact same protocol described above. The experiment showed an even more prominent activity in ZR-75 cells than in HEK293 cells, with both IC₂₅ and IC₅₀ values in the sub-micromolar range.

### * * *

1. A compound having general formula (I): 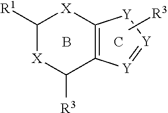
wherein:
ring B may be partially saturated, for instance a di-hydropyridinone ring when R¹ is O and/or when one or both X is N, or insaturated, e.g. a pyridinone ring when R¹ is O;
R¹ may be 0 or S;
each atom on ring B may be independently a C or N atom;
X may be independently selected from —C— or —O— or —N(R⁴)—, wherein R⁴ may be —H, or —C₁₋₄ linear or cyclic alkyl;
ring C is a 5- or 6-membered aromatic ring wherein each atom may be —C— or —N—, e.g. Y may independently be —C(R³) or —N(R³)— or —N═;
R³ is selected from —H, C₁₋₄ linear or cyclic alkyl group, alkoxyl or aryloxyl, ring A or Z-ring A;
preferably, ring C has one or two heteroatoms;
Z may be —CH₂— or —O— or —N(H)— or —S(O₂)— or —S(O)—;
ring A may be an aromatic or heteroaromatic 5- or 6-membered ring, preferably a phenyl ring; ring A may be substituted at any position with one or more group R² independently selected from: —H, —F, —Cl, —Br, —I, —CH₃, —CH₂CH₃, —CH(CH₃)₂, —C(CH₃)₃, —CHF₂, —CF₃, —OH, —OCH₃,
—OCH₂CH₃, —OCF₃, —OCH(CH₃)₂, —CN, —NO₂, —NH₂, —SH;
or general formula (II): 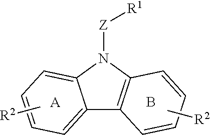
wherein:
each atom on each ring may be independently a —C— or —N— atom;
Z may be —H, —CH₂—, —S(O₂)—, —S(O)—, R¹ may be absent (when Z is —H) or may be a group independently selected from: H, C₁₋₄ linear, branched, cyclic alkyl or alkoxyl or aryloxyl optionally substituted with one or more of: —H, —F, —Cl,
—Br, —I, —CH₃, —CH₂CH₃, —CH(CH₃)₂, —C(CH₃)₃, —CHF₂, —CF₃,
—OH, —OCH₃, —OCH₂CH₃, —OCF₃, —OCH(CH₃)₂, —CN, —NO₂, —NH₂, —S, a tetrahydrofuran ring or a tetrahydropyran ring wherein each atom of the ring may independently be substituted with —H or —OH, an aromatic or heteroaromatic 5- or 6-membered ring substituted at any position with one or more group R² independently selected from: —H, —F, —Cl, —Br, —I, —CH₃, —CH₂CH₃,
—CH(CH₃)₂, —C(CH₃)₃, —CHF₂, —CF₃, —OH, —OCH₃, —OCH₂CH₃,
—OCF₃, —OCH(CH₃)₂, —CN, —NO₂, —NH₂, —SH;
ring A and ring B are each independently an aromatic or heteroaromatic 5- or 6-membered ring, preferably a phenyl ring; ring A and ring B may be independently substituted at any position with one or more group R² independently selected from: —H, —F, —Cl, —Br, —I, —CH₃, —CH₂CH₃, —CH(CH₃)₂, —C(CH₃)₃, —CHF₂, —CF₃, —OH, —OCH₃,
—OCH₂CH₃, —OCF₃, —OCH(CH₃)₂, —CN, —NO₂, —NH₂, —SH;
or general formula (III): 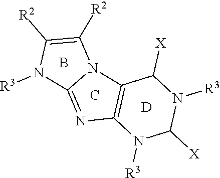
wherein:
each atom on each aromatic ring may be independently a C or N atom;
ring B and ring C are fused imidazole rings,
ring D is a pyrimidine ring, optionally ring D may be a cytosine or uracyl ring;
R² are each independently —H, —OH, —CH₂OH, —CH₃, —CH₂CH₃, halogen selected in the group comprising: —F, —Cl, —Br, —I, —CF₃, —OCH₃;
X may be independently ═O, —NH₂, ═S;
R³ is independently selected from —H, C₁₋₄ linear, branched or cyclic alkyl, ring A or Z-ring A;
Z may be —CH₂ or —S(O₂)— or —S(O)—,
ring A may be an aromatic or heteroaromatic 5- or 6-membered ring, preferably a phenyl ring; ring A may be substituted at any position with one or more group R¹ independently selected from: —H, —F, —Cl, —Br, —I, —CH₃, —CH₂CH₃, —CH(CH₃)₂, —C(CH₃)₃, —CHF₂, —CF₃, —OH, —OCH₃,
—OCH₂CH₃, —OCF₃, —OCH(CH₃)₂, —CN, —NO₂, —NH₂, —SH;
or general formula (IV): 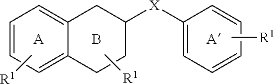
wherein:
each atom on each ring may be independently a C or N atom;
ring A and A′ are each independently an aromatic or heteroaromatic 5- or 6-membered ring, preferably a phenyl ring;
ring A, A′ and B may be substituted at any position with one or more group R¹ independently selected from: —H,
—F, —Cl, —Br, —I, —CH₃, —CH₂CH₃, —CH(CH₃)₂, —C(CH₃)₃,
—CHF₂, —CF₃, —OH, —OCH₃, —OCH₂CH₃, —OCF₃, —OCH(CH₃)₂, —CN, —NO₂, —NH₂, —SH;
ring B is an aromatic or a non aromatic ring wherein each atom may be independently one or more C or N or O or S or S(O₂) atom and preferably is a piperidine ring;
X is —CH₂, —O—, —S—, —N(H), —C(O)—, —C(S)—, —C(H)═C(H)—, —S(O₂), —S(O).

2. The compound according to claim 1, having formula (I.1): 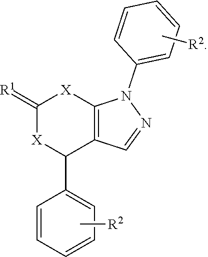
wherein each atom on ring B may be independently a C or N atom,
X may be independently selected from —C— or —O— or N(R⁴), R⁴ may be —H, or —C₁₋₄ linear or cyclic alkyl,
R¹ may be O or S,
R² may be independently selected from: —H, —F, —Cl, —Br, —I, —CH₃, —CH₂CH₃, —CH(CH₃)₂, —C(CH₃)₃, —CHF₂, —CF₃, —OH,
—OCH₃, —OCH₂CH₃, —OCF₃, —OCH(CH₃)₂, —CN, —NO₂, —NH₂, —SH, wherein one or two or three R² may be present on each one of Ring A or Ring D.

3. The compound according to claim 1 having formula:

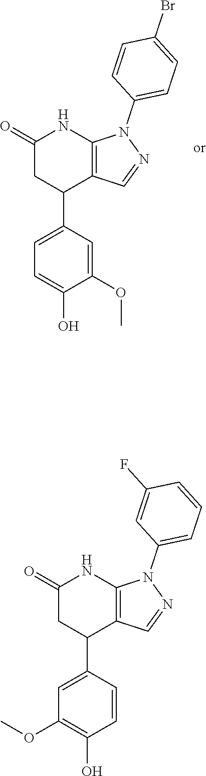 [SMILES: COc1cc(C2CC(=O)Nc3c2cnn3-c2ccc(Br)cc2)ccc1O ; COc1cc(C2CC(=O)Nc3c2cnn3-c2cccc(F)c2)ccc1O]

4. The compound according to claim 1 having formula:

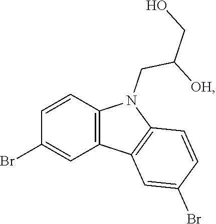 [SMILES: OCC(O)Cn1c2ccc(Br)cc2c2cc(Br)ccc21]

5. The compound according to claim 1 having formula (III.1) 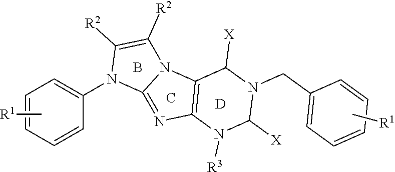
wherein each atom on each aromatic ring may be independently a C or N atom, ring B and ring C are fused imidazole rings,
ring D is a pyrimidine ring, optionally ring D may be a cytosine or uracyl ring;
X may be independently ═O, —NH₂, ═S;
R¹ may independently selected from: —H, —F, —Cl, —Br, —I, —CH₃, —CH₂CH₃, —CH(CH₃)₂, —C(CH₃)₃, —CHF₂, —CF₃, —OH,
—OCH₃, —OCH₂CH₃, —OCF₃, —OCH(CH₃)₂, —CN, —NO₂, —NH₂, —SH;
R² may be each independently —H, —OH, —CH₂OH, —CH₃,
—CH₂CH₃, halogen selected in the group comprising: —F,
—Cl, —Br, —I, —CF₃, —OCH₃,
R³ is independently selected from —H, C₁₋₄ linear, branched or cyclic alkyl, ring A or Z-ring A,
Z may be —CH₂ or —S(O₂)— or —S(O)—,
ring A may be an aromatic or heteroaromatic 5- or 6-membered ring, preferably a phenyl ring; ring A may be substituted at any position with one or more group R¹.

6. The compound according to claim 1 having formula:

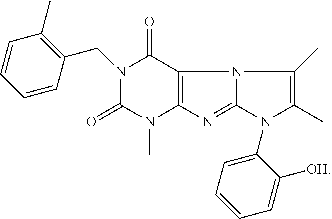 [SMILES: Cc1ccccc1Cn1c(=O)c2c(nc3n(-c4ccccc4O)c(C)c(C)n23)n(C)c1=O]

7. The compound according to claim 1 having formula (IV.1): 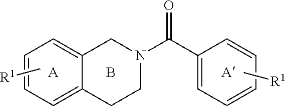
ring A and A′ may be each independently an aromatic or heteroaromatic 5- or 6-membered ring, preferably a phenyl ring,
ring B may be an aromatic or a non aromatic ring wherein each atom may be independently one or more C or N or O or S or S(O₂) atom and preferably is a piperidine ring, ring A, A′ and B may be substituted at any position with one or more group R¹,
R¹ may be independently selected from: —H, —F, —Cl, —Br, —I, —CH₃, —CH₂CH₃, —CH(CH₃)₂, —C(CH₃)₃, —CHF₂, —CF₃, —OH,
—OCH₃, —OCH₂CH₃, —OCF₃, —OCH(CH₃)₂, —CN, —NO₂, —NH₂, —SH.

8. The compound according to claim 1 having formula:

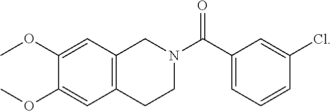 [SMILES: COc1cc2c(cc1OC)CN(C(=O)c1cccc(Cl)c1)CC2]

9. A compound having formula 1.2: 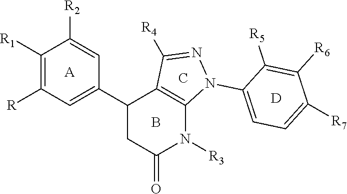
wherein Ring A and Ring D may independently be aromatic or heteroaromatic rings, wherein the heteroatoms are one, two or three and preferably are one or two and preferably are represented by N,
and wherein Ring A is selected from: 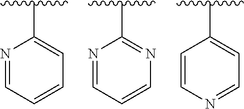
and wherein Ring D is selected from: 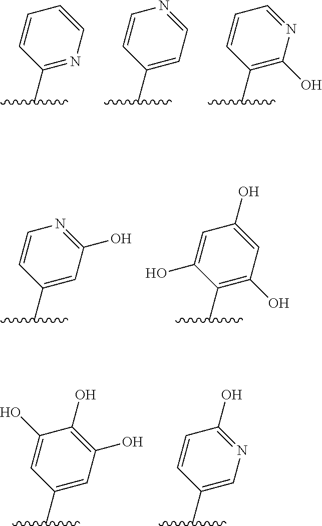
wherein R, R₁ and R₂ may independently be H, hydroxyl, C₁₋₃ alkoxyl group, phenoxyl, benzyloxyl, or R and R₁ form a 1,4-dioxane ring, or R₁ is phenoxyl or benzyloxyl or R₁ is selected from: 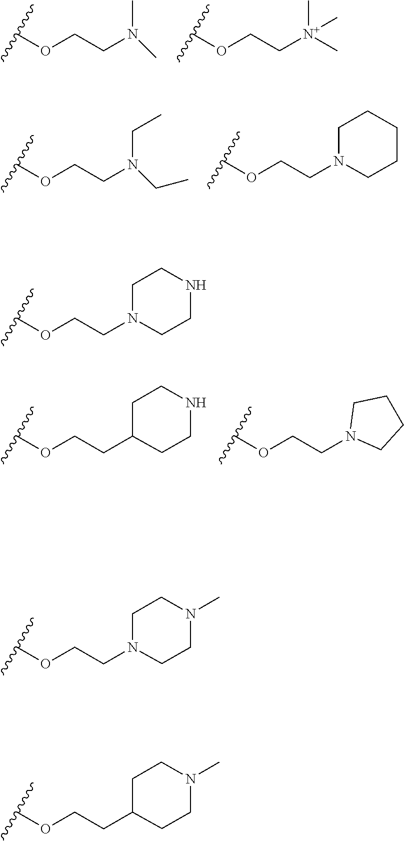
R₃ may be H, C₁₋₃ alkyl and preferably H or methyl,
R₄ may be H, C₁₋₃ alkyl and preferably H or methyl,
R₅, R₆, R₇ may independently be: H, hydroxyl, halide, methyl, triflouromethyl, hydroxyl, Ring D may have one, two or three substituents; and R₅ and R₆ are H and preferably, when R₅ and R₆ are H, R₇ is I or Br.

10. The compound according to claim 9, wherein:
Ring A and Ring D are aromatic rings;
R, R₁ and R₂ may be H, hydroxyl, C_{1-C3} alkoxyl, phenoxyl or benzyloxyl or R and R₁ form a 1,4-dioxane ring,
R₃ may be H, C₁₋₃ alkyl and is preferably represented by H or methyl, R₄ may be H, C₁₋₃ alkyl and is preferably represented by H or methyl, R₅, R₆, R₇ may independently be H, halide, methyl, triflouromethyl,
Ring D may have one, two or three substituents, preferably R₅ and R₆ are H and preferably when R₅ and R₆ are H, R₇ is I or Br.

11. The compound according to claim 2, wherein Ring A and Ring D are each a phenyl ring;
R, R₁ and R₂ may be H, hydroxyl, C₁₋₃ alkoxyl group,
R₃ may be H, C₁₋₃ alkyl and is preferably represented by H or methyl,
R₄ may be C₁₋₃ alkyl and preferably methyl,
R₅, R₆, R₇ may independently be H, halide, methyl, triflouromethyl,
Ring D may have one, two or three substituents, preferably R₅ and R₆ are H and preferably when R₅ and R₆ are H, R₇ is I or Br.

12. A compound according to claim 2 wherein:
Ring A and Ring D are each a phenyl ring,
when R is —OH, R₁ is —OCH₃,
when R is —OCH₃, R₁ is —OH,
when R is —H, —OPh or —OCH₂Ph,
when R is —OCH₂CH₃, R₁ is —O—CH₃,
R and R₁ form a 1,4-dioxane ring,
R₄ is H, or methyl,
R₅ is —H,
R₆ is —H,
R₇ is —H, —Br, —F, —I, trifluoromethyl, —Cl.

13. A compound having one of the following formula:

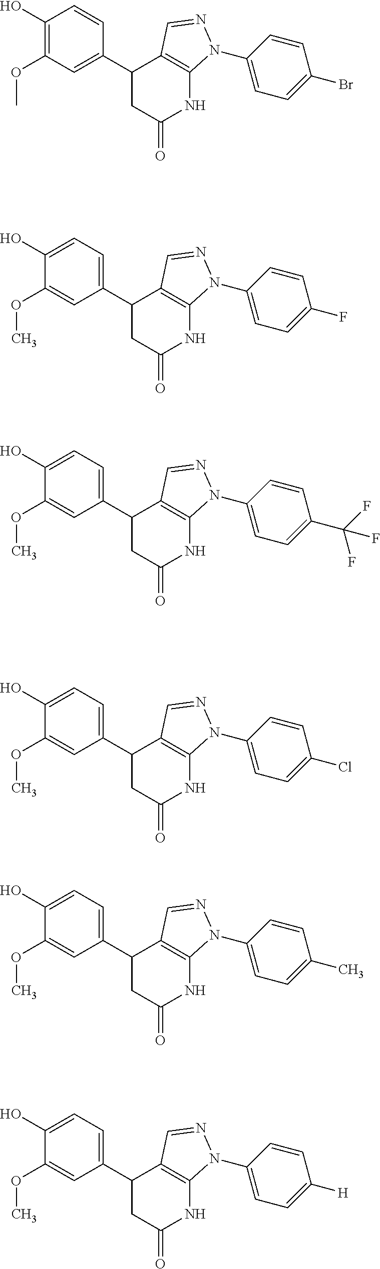 [SMILES: COc1cc(C2CC(=O)Nc3c2cnn3-c2ccc(Br)cc2)ccc1O ; COc1cc(C2CC(=O)Nc3c2cnn3-c2ccc(F)cc2)ccc1O ; COc1cc(C2CC(=O)Nc3c2cnn3-c2ccc(C(F)(F)F)cc2)ccc1O ; COc1cc(C2CC(=O)Nc3c2cnn3-c2ccc(Cl)cc2)ccc1O ; COc1cc(C2CC(=O)Nc3c2cnn3-c2ccc(C)cc2)ccc1O ; COc1cc(C2CC(=O)Nc3c2cnn3-c2ccccc2)ccc1O]

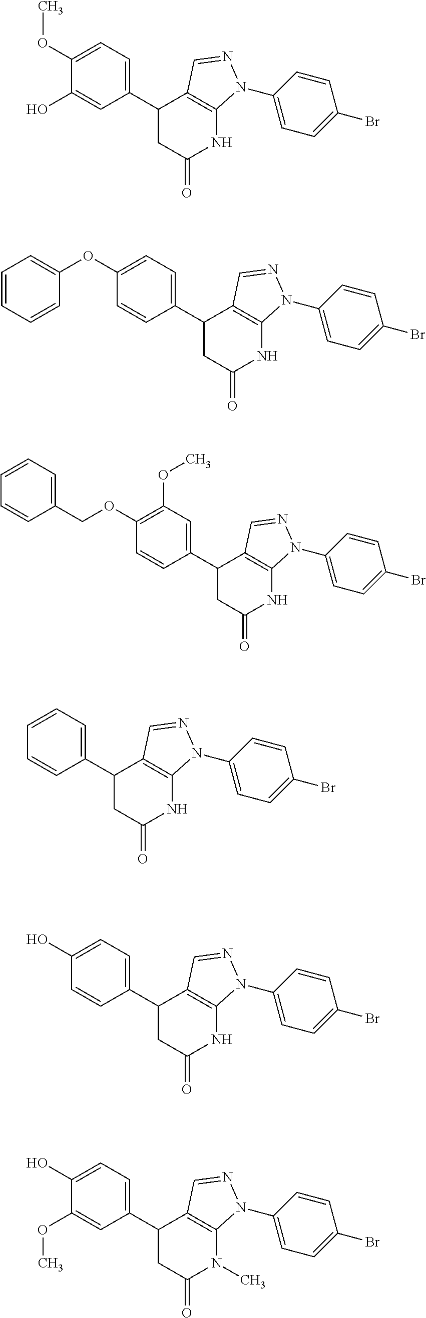 [SMILES: COc1ccc(C2CC(=O)Nc3c2cnn3-c2ccc(Br)cc2)cc1O ; O=C1CC(c2ccc(Oc3ccccc3)cc2)c2cnn(-c3ccc(Br)cc3)c2N1 ; COc1cc(C2CC(=O)Nc3c2cnn3-c2ccc(Br)cc2)ccc1OCc1ccccc1 ; O=C1CC(c2ccccc2)c2cnn(-c3ccc(Br)cc3)c2N1 ; O=C1CC(c2ccc(O)cc2)c2cnn(-c3ccc(Br)cc3)c2N1 ; COc1cc(C2CC(=O)N(C)c3c2cnn3-c2ccc(Br)cc2)ccc1O]

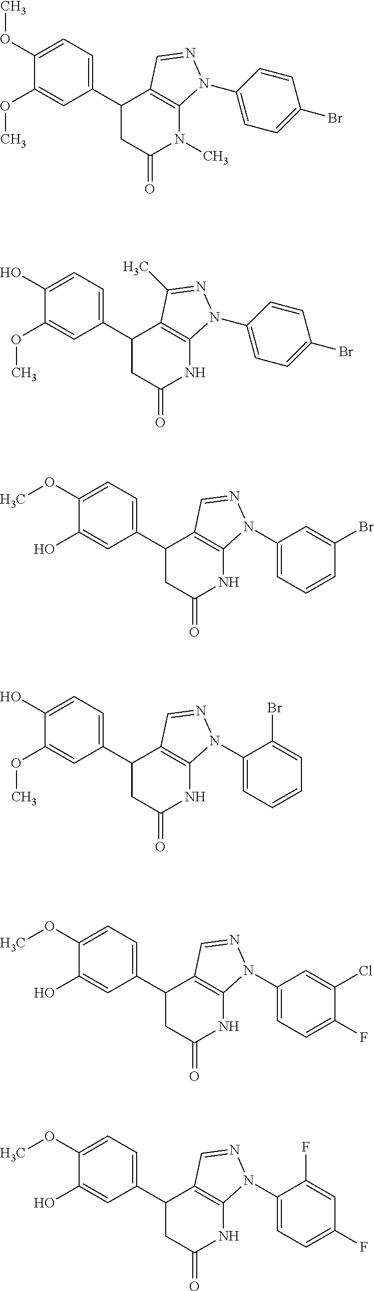 [SMILES: COc1ccc(C2CC(=O)N(C)c3c2cnn3-c2ccc(Br)cc2)cc1OC ; COc1cc(C2CC(=O)Nc3c2c(C)nn3-c2ccc(Br)cc2)ccc1O ; COc1ccc(C2CC(=O)Nc3c2cnn3-c2cccc(Br)c2)cc1O ; COc1cc(C2CC(=O)Nc3c2cnn3-c2ccccc2Br)ccc1O ; COc1ccc(C2CC(=O)Nc3c2cnn3-c2ccc(F)c(Cl)c2)cc1O ; COc1ccc(C2CC(=O)Nc3c2cnn3-c2ccc(F)cc2F)cc1O]

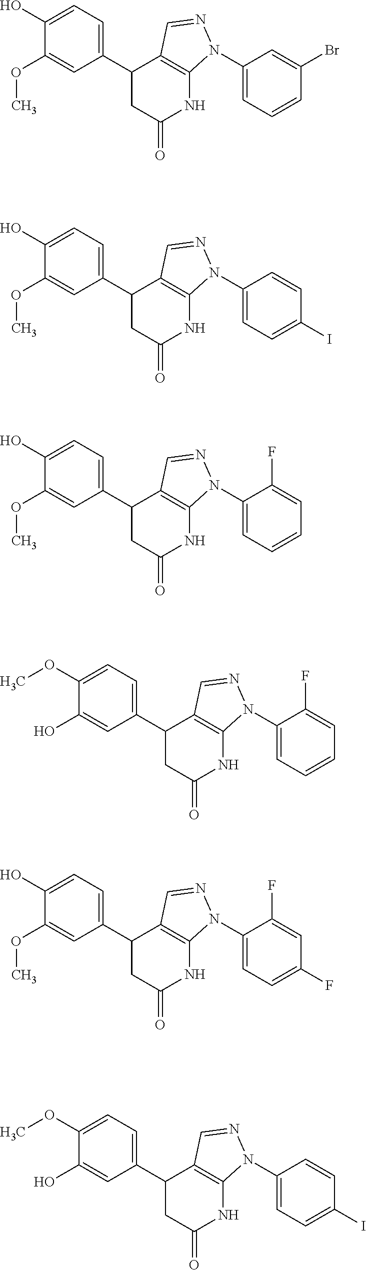 [SMILES: COc1cc(C2CC(=O)Nc3c2cnn3-c2cccc(Br)c2)ccc1O ; COc1cc(C2CC(=O)Nc3c2cnn3-c2ccc(I)cc2)ccc1O ; COc1cc(C2CC(=O)Nc3c2cnn3-c2ccccc2F)ccc1O ; COc1ccc(C2CC(=O)Nc3c2cnn3-c2ccccc2F)cc1O ; COc1cc(C2CC(=O)Nc3c2cnn3-c2ccc(F)cc2F)ccc1O ; COc1ccc(C2CC(=O)Nc3c2cnn3-c2ccc(I)cc2)cc1O]

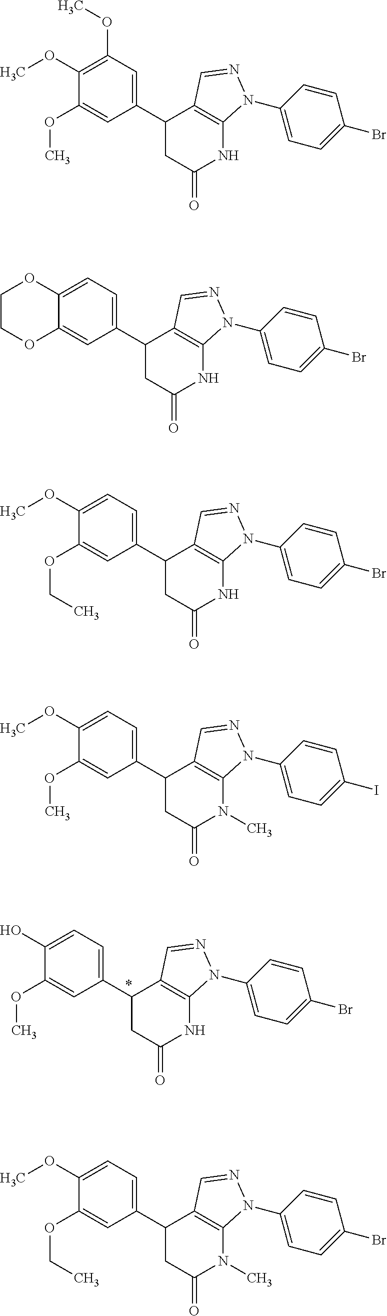 [SMILES: COc1cc(C2CC(=O)Nc3c2cnn3-c2ccc(Br)cc2)cc(OC)c1OC ; O=C1CC(c2ccc3c(c2)OCCO3)c2cnn(-c3ccc(Br)cc3)c2N1 ; CCOc1cc(C2CC(=O)Nc3c2cnn3-c2ccc(Br)cc2)ccc1OC ; COc1ccc(C2CC(=O)N(C)c3c2cnn3-c2ccc(I)cc2)cc1OC ; COc1cc(C2CC(=O)Nc3c2cnn3-c2ccc(Br)cc2)ccc1O ; CCOc1cc(C2CC(=O)N(C)c3c2cnn3-c2ccc(Br)cc2)ccc1OC]

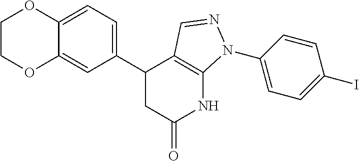 [SMILES: O=C1CC(c2ccc3c(c2)OCCO3)c2cnn(-c3ccc(I)cc3)c2N1]

14. The compound according to claim 13, which is represented by an enantiomeric form of said compounds.

15. The compound according to claim 13 being represented by the R-enantiomer of compound of formula:

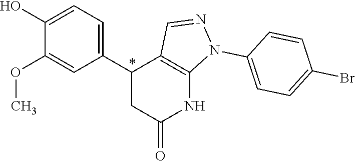 [SMILES: COc1cc(C2CC(=O)Nc3c2cnn3-c2ccc(Br)cc2)ccc1O]

16. A pharmaceutical composition comprising one or more of the compounds according to claim 1.

17. A compound according to claim 1 for use as a medicament.

18. A compound according to claim 17 for use as a medicament in the treatment of neurodegenerative disorders, neuroinflammatory disorders, demyelinating diseases and cancer.

19. A compound according to claim 18 wherein said neurodegenerative disorders are selected in the group comprising sporadic, inherited or acquired prion diseases, Alzheimer's disease, Parkinson's diseases and other α-synucleinopathies; said neuroinflammatory disorders and demyelinating diseases comprising multiple sclerosis; said cancer is selected in the group comprising glioblastoma, gastric cancer, breast cancer, colon cancer.

20. A compound according to claim 19 wherein the prion disease comprises: Creutzfeldt-Jakob disease (CJD), Gerstmann-Sträussler-Scheinker (GSS) syndrome and fatal familial insomnia (FFI).

21. A compound according to claim 17, for use as a medicament in combination with one or more other medicaments.

22. A compound according to claim 1 for use as a medicament in combination with one or more other medicaments selected from the group comprising: small molecules, antibodies, peptide-based therapeutics, RNA-based therapeutics gene therapy or gene editing therapeutic approaches.

23. A compound according to claim 22 wherein said one or more other medicaments are against diseases or disorders related to the cellular prion protein (PrP^{C}) or interactors of PrP^{C} or toxic signaling pathways involving PrP^{C}.

24. The compound according to claim 23 wherein said diseases or disorders related to the cellular prion protein (PrP^{C}) or interactors of PrP^{C} or toxic signaling pathways involving PrP^{C} comprise neurodegenerative disorders, such as sporadic, inherited or acquired prion diseases, Alzheimer's disease, Parkinson's diseases and other α-synucleinopathies; neuroinflammatory disorders and demyelinating diseases, such as multiple sclerosis; cancer, in particular glioblastoma, gastric cancer, breast cancer, colon cancer.

25. A method for the treatment of diseases or disorders related to the cellular prion protein (PrP^{C}) or interactors of PrP^{C} or toxic signaling pathways involving PrP^{C} comprising the administration to a patient in need thereof of a compound according to claim 1.

26. The method according to claim 25 wherein said diseases or disorders related to the cellular prion protein (PrP^{C}) or interactors of PrP^{C} or toxic signaling pathways involving PrP^{C} comprise neurodegenerative disorders, such as sporadic, inherited or acquired prion diseases, Alzheimer's disease, Parkinson's diseases and other α-synucleinopathies; neuroinflammatory disorders and demyelinating diseases, such as multiple sclerosis; cancer, in particular glioblastoma, gastric cancer, breast cancer, colon cancer.

27. The method according to claim 24 further comprises the administration of one or more other medicaments.

28. The method according to claim 27 wherein said one or more other medicaments are selected from the group comprising: small molecules, antibodies, peptide-based therapeutics, RNA-based therapeutics gene therapy or gene editing therapeutic approaches.

29. The method according to claim 28, wherein said one or more other medicaments are against diseases or disorders related to the cellular prion protein (PrP^{C}) or interactors of PrP^{C} or toxic signaling pathways involving PrP^{C}.
